SEQUENCE LISTING (116 sequences)

SEQ ID NO: 1 - protein, 16 aa, synthetic construct
  REGION 1..16: note = Description of Artificial Sequence: Synthetic peptide
DKTHTCPPCPAPELLG

SEQ ID NO: 2 - protein, 15 aa, synthetic construct
  REGION 1..15: note = Description of Artificial Sequence: Synthetic peptide
DKTHTCPPCPAPPVA

SEQ ID NO: 3 - protein, 15 aa, synthetic construct
  REGION 1..15: note = Description of Artificial Sequence: Synthetic peptide
DKTHTCPPCPAPGGG

SEQ ID NO: 4 - protein, 17 aa, synthetic construct
  REGION 1..17: note = Description of Artificial Sequence: Synthetic peptide
ERKCCVECPPCPAPPVA

SEQ ID NO: 5 - protein, 12 aa, synthetic construct
  REGION 1..12: note = Description of Artificial Sequence: Synthetic peptide
VECPPCPAPPVA

SEQ ID NO: 6 - protein, 21 aa, synthetic construct
  REGION 1..21: note = Description of Artificial Sequence: Synthetic peptide
EPKSCDTPPPCPPCPAPELLG

SEQ ID NO: 7 - protein, 18 aa, synthetic construct
  REGION 1..18: note = Description of Artificial Sequence: Synthetic peptide
ESKYGPPCPPCPAPEFLG

SEQ ID NO: 8 - protein, 17 aa, synthetic construct
  REGION 1..17: note = Description of Artificial Sequence: Synthetic peptide
ESKYGPPCPPCPAPPVA

SEQ ID NO: 9 - protein, 17 aa, synthetic construct
  REGION 1..17: note = Description of Artificial Sequence: Synthetic peptide
ESKYGPPCPPCPAPGGG

SEQ ID NO: 10 - protein, 15 aa, synthetic construct
  REGION 1..15: note = Description of Artificial Sequence: Synthetic peptide
DKTHTCPPCPAPPVA

SEQ ID NO: 11 - protein, 16 aa, synthetic construct
  REGION 1..16: note = Description of Artificial Sequence: Synthetic peptide
DKTHTCPPCPGGGFLG

SEQ ID NO: 12 - protein, 16 aa, synthetic construct
  REGION 1..16: note = Description of Artificial Sequence: Synthetic peptide
DKTHTCPPCPGGGGLG

SEQ ID NO: 13 - protein, 16 aa, synthetic construct
  REGION 1..16: note = Description of Artificial Sequence: Synthetic peptide
DKTHTCPPCPGGGLLG

SEQ ID NO: 14 - protein, 13 aa, synthetic construct
  REGION 1..13: note = Description of Artificial Sequence: Synthetic peptide
DKTHTCPPCPLLG

SEQ ID NO: 15 - protein, 35 aa, synthetic construct
  REGION 1..35: note = Description of Artificial Sequence: Synthetic polypeptide
EPKSCDKTHTCPPCPAPELLGGPSVFLFPPKPKDT

SEQ ID NO: 16 - protein, 34 aa, synthetic construct
  REGION 1..34: note = Description of Artificial Sequence: Synthetic polypeptide
EPKSCDKTHTCPPCPAPPVAGPSVFLFPPKPKDT

SEQ ID NO: 17 - protein, 34 aa, synthetic construct
  REGION 1..34: note = Description of Artificial Sequence: Synthetic polypeptide
EPKSCDKTHTCPPCPAPGGGGPSVFLFPPKPKDT

SEQ ID NO: 18 - protein, 31 aa, synthetic construct
  REGION 1..31: note = Description of Artificial Sequence: Synthetic polypeptide
ERKCCVECPPCPAPPVAGPSVFLFPPKPKDT

SEQ ID NO: 19 - protein, 35 aa, synthetic construct
  REGION 1..35: note = Description of Artificial Sequence: Synthetic polypeptide
EPKSCDTPPPCPPCPAPELLGGPSVFLFPPKPKDT

SEQ ID NO: 20 - protein, 32 aa, synthetic construct
  REGION 1..32: note = Description of Artificial Sequence: Synthetic polypeptide
ESKYGPPCPPCPAPEFLGGPSVFLFPPKPKDT

SEQ ID NO: 21 - protein, 31 aa, synthetic construct
  REGION 1..31: note = Description of Artificial Sequence: Synthetic polypeptide
ESKYGPPCPPCPAPPVAGPSVFLFPPKPKDT

SEQ ID NO: 22 - protein, 31 aa, synthetic construct
  REGION 1..31: note = Description of Artificial Sequence: Synthetic polypeptide
ESKYGPPCPPCPAPGGGGPSVFLFPPKPKDT

SEQ ID NO: 23 - protein, 211 aa, synthetic construct
  REGION 1..211: note = Description of Artificial Sequence: Synthetic polypeptide
GPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 24 - protein, 211 aa, synthetic construct
  REGION 1..211: note = Description of Artificial Sequence: Synthetic polypeptide
GPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 25 - protein, 211 aa, synthetic construct; identical to SEQ ID NO: 23
  REGION 1..211: note = Description of Artificial Sequence: Synthetic polypeptide

SEQ ID NO: 26 - protein, 211 aa, synthetic construct
  REGION 1..211: note = Description of Artificial Sequence: Synthetic polypeptide
GPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 27 - protein, 211 aa, synthetic construct; identical to SEQ ID NO: 23
  REGION 1..211: note = Description of Artificial Sequence: Synthetic polypeptide

SEQ ID NO: 28 - protein, 211 aa, synthetic construct
  REGION 1..211: note = Description of Artificial Sequence: Synthetic polypeptide
GPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 29 - protein, 211 aa, synthetic construct; identical to SEQ ID NO: 28
  REGION 1..211: note = Description of Artificial Sequence: Synthetic polypeptide

SEQ ID NO: 30 - protein, 211 aa, synthetic construct; identical to SEQ ID NO: 28
  REGION 1..211: note = Description of Artificial Sequence: Synthetic polypeptide

SEQ ID NO: 31 - protein, 211 aa, synthetic construct; identical to SEQ ID NO: 28
  REGION 1..211: note = Description of Artificial Sequence: Synthetic polypeptide

SEQ ID NO: 32 - protein, 222 aa, synthetic construct
  REGION 1..222: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKERKCCVECPPCPAPPVA

SEQ ID NO: 33 - protein, 217 aa, synthetic construct
  REGION 1..217: note = Description of Artificial Sequence: Synthetic polypeptide
  VARIANT 36: note = May be N-glycosylated
  VARIANT 68: note = May be N-glycosylated
  VARIANT 123: note = May be N-glycosylated
  VARIANT 196: note = May be N-glycosylated
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKVECPPCPAPPVA

SEQ ID NO: 34 - protein, 220 aa, synthetic construct
  REGION 1..220: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPPVA

SEQ ID NO: 35 - protein, 222 aa, synthetic construct
  REGION 1..222: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKESKYGPPCPPCPAPPVA

SEQ ID NO: 36 - protein, 220 aa, synthetic construct; identical to SEQ ID NO: 34
  REGION 1..220: note = Description of Artificial Sequence: Synthetic polypeptide

SEQ ID NO: 37 - protein, 221 aa, synthetic construct
  REGION 1..221: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPGGGFLG

SEQ ID NO: 38 - protein, 221 aa, synthetic construct
  REGION 1..221: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPGGGGLG

SEQ ID NO: 39 - protein, 221 aa, synthetic construct
  REGION 1..221: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPGGGLLG

SEQ ID NO: 40 - protein, 218 aa, synthetic construct
  REGION 1..218: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPLLG

SEQ ID NO: 41 - protein, 428 aa, synthetic construct
  REGION 1..428: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 42 - protein, 431 aa, synthetic construct
  REGION 1..431: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 43 - protein, 433 aa, synthetic construct
  REGION 1..433: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 44 - protein, 433 aa, synthetic construct
  REGION 1..433: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 45 - protein, 431 aa, synthetic construct
  REGION 1..431: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 46 - protein, 432 aa, synthetic construct
  REGION 1..432: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPGGGFLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 47 - protein, 432 aa, synthetic construct
  REGION 1..432: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPGGGGLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 48 - protein, 432 aa, synthetic construct
  REGION 1..432: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPGGGLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 49 - protein, 429 aa, synthetic construct
  REGION 1..429: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 50 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 51 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 52 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 53 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 54 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 55 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 56 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 57 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 58 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 59 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 60 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 61 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSNGNLKAIYVTDSDSNASIGMKKYFVGVNSAGKVAISAKEIKEDNIGAQVLGLFTLSTGQDSWNQTN

SEQ ID NO: 62 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFINGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 63 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFNGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 64 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFNGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 65 - protein, 339 aa, synthetic construct
  REGION 1..339: note = Description of Artificial Sequence: Synthetic polypeptide
MRKRCYSTSAAVLAAVTLFVLSVDRGVIADSFSANQEIRYSEVTPYHVTSVWTKGVTPPANFTQGEDVFHAPYVANQGWYDITKTFDGKDDLLCGAATAGNMLHWWFDQNKDQIKRYLEEHPEKQKINFRGEQMFDVKEAIDTKNHQLDSKLFEYFKEKAFPYLSTKHLGVFPDHVIDMFILGYRLSLTNHGPTPVKEGSKDPRGGIFDAVFTRGDQSKLLTSRHDFKEKNLKEISDLIKKELTEGKALGLSHTYANVRINHVINLWGADFDSDGNLKAIYVTDSDSNASIGMKKYFVGVNSAGKVAISAKEIKEDNIGAQVLGLFTLSTGQDSWNQTN

SEQ ID NO: 66 - protein, 339 aa, Streptococcus pyogenes; identical to SEQ ID NO: 50

SEQ ID NO: 67 - protein, 221 aa, synthetic construct
  REGION 1..221: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLG

SEQ ID NO: 68 - protein, 16 aa, synthetic construct
  REGION 1..16: note = Description of Artificial Sequence: Synthetic peptide
DKTHTCPLCPAPELLG

SEQ ID NO: 69 - protein, 432 aa, synthetic construct
  REGION 1..432: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 70 - protein, 19 aa, synthetic construct
  REGION 1..19: note = Description of Artificial Sequence: Synthetic peptide
DKTHTCPPCPAPPVAGPSV

SEQ ID NO: 71 - DNA, 669 bp, synthetic construct
  misc_feature 1..669: note = Description of Artificial Sequence: Synthetic polynucleotide
  source 1..669: mol_type = other DNA
agtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggagcgcaaatgttgtgtcgagtgcccaccgtgcccagcaccacctgtggcatga

SEQ ID NO: 72 - DNA, 654 bp, synthetic construct
  misc_feature 1..654: note = Description of Artificial Sequence: Synthetic polynucleotide
  source 1..654: mol_type = other DNA
agtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggtcgagtgcccaccgtgcccagcaccacctgtggcatga

SEQ ID NO: 73 - DNA, 666 bp, synthetic construct
  misc_feature 1..666: note = Description of Artificial Sequence: Synthetic polynucleotide
  source 1..666: mol_type = other DNA
agtgataccggtagacctttcgtagagatgtacagtgaaatccccgaaattatacacatgactgaaggaagggagctcgtcattccctgccgggttacgtcacctaacatcactgttactttaaaaaagtttccacttgacactttgatccctgatggaaaacgcataatctgggacagtagaaagggcttcatcatatcaaatgcaacgtacaaagaaatagggcttctgacctgtgaagcaacagtcaatgggcatttgtataagacaaactatctcacacatcgacaaaccaatacaatcatagatgtggttctgagtccgtctcatggaattgaactatctgttggagaaaagcttgtcttaaattgtacagcaagaactgaactaaatgtggggattgacttcaactgggaatacccttcttcgaagcatcagcataagaaacttgtaaaccgagacctaaaaacccagtctgggagtgagatgaagaaatttttgagcaccttaactatagatggtgtaacccggagtgaccaaggattgtacacctgtgcagcatccagtgggctgatgaccaagaagaacagcacatttgtcagggtccatgaaaaggacaaaactcacacatgcccaccgtgcccaggcggtggacttctagggtga

SEQ ID NO: 74 - protein, 35 aa, synthetic construct
  REGION 1..35: note = Description of Artificial Sequence: Synthetic polypeptide
EPKSCDKTHTCPPCPGGGFLGGPSVFLFPPKPKDT

SEQ ID NO: 75 - protein, 35 aa, synthetic construct
  REGION 1..35: note = Description of Artificial Sequence: Synthetic polypeptide
EPKSCDKTHTCPPCPGGGGLGGPSVFLFPPKPKDT

SEQ ID NO: 76 - protein, 35 aa, synthetic construct
  REGION 1..35: note = Description of Artificial Sequence: Synthetic polypeptide
EPKSCDKTHTCPPCPGGGLLGGPSVFLFPPKPKDT

SEQ ID NO: 77 - protein, 32 aa, synthetic construct
  REGION 1..32: note = Description of Artificial Sequence: Synthetic polypeptide
EPKSCDKTHTCPPCPLLGGPSVFLFPPKPKDT

SEQ ID NO: 78 - protein, 223 aa, synthetic construct
  REGION 1..223: note = Description of Artificial Sequence: Synthetic polypeptide
VECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 79 - protein, 226 aa, synthetic construct
  REGION 1..226: note = Description of Artificial Sequence: Synthetic polypeptide
DKTHTCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 80 - protein, 228 aa, synthetic construct
  REGION 1..228: note = Description of Artificial Sequence: Synthetic polypeptide
ERKCCVECPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 81 - protein, 228 aa, synthetic construct
  REGION 1..228: note = Description of Artificial Sequence: Synthetic polypeptide
ESKYGPPCPPCPAPPVAGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 82 - protein, 226 aa, synthetic construct
  REGION 1..226: note = Description of Artificial Sequence: Synthetic polypeptide
DKTHTCPPCPAPPVAGPSVFLFPPKPKDTLMISRTPEVTCVVVDVSHEDPEVQFNWYVDGVEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 83 - protein, 227 aa, synthetic construct
  REGION 1..227: note = Description of Artificial Sequence: Synthetic polypeptide
DKTHTCPPCPGGGFLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 84 - protein, 227 aa, synthetic construct
  REGION 1..227: note = Description of Artificial Sequence: Synthetic polypeptide
DKTHTCPPCPGGGGLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 85 - protein, 227 aa, synthetic construct
  REGION 1..227: note = Description of Artificial Sequence: Synthetic polypeptide
DKTHTCPPCPGGGLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 86 - protein, 224 aa, synthetic construct
  REGION 1..224: note = Description of Artificial Sequence: Synthetic polypeptide
DKTHTCPPCPLLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 87 - protein, 12 aa, synthetic construct
  REGION 1..12: note = Description of Artificial Sequence: Synthetic peptide
DKTHTCPPCPPC

SEQ ID NO: 88 - protein, 15 aa, synthetic construct
  REGION 1..15: note = Description of Artificial Sequence: Synthetic peptide
DKTHTCPPCPPCPPC

SEQ ID NO: 89 - protein, 16 aa, synthetic construct
  REGION 1..16: note = Description of Artificial Sequence: Synthetic peptide
DKTHTCPLCPAPELLG

SEQ ID NO: 90 - protein, 4 aa, synthetic construct
  REGION 1..4: note = Description of Artificial Sequence: Synthetic peptide
ELLG

SEQ ID NO: 91 - protein, 4 aa, synthetic construct
  REGION 1..4: note = Description of Artificial Sequence: Synthetic peptide
APEL

SEQ ID NO: 92 - protein, 4 aa, synthetic construct
  REGION 1..4: note = Description of Artificial Sequence: Synthetic peptide
GGGF

SEQ ID NO: 93 - protein, 4 aa, synthetic construct
  REGION 1..4: note = Description of Artificial Sequence: Synthetic peptide
GGGG

SEQ ID NO: 94 - protein, 4 aa, synthetic construct
  REGION 1..4: note = Description of Artificial Sequence: Synthetic peptide
GGGL

SEQ ID NO: 95 - protein, 4 aa, synthetic construct
  REGION 1..4: note = Description of Artificial Sequence: Synthetic peptide
EFLG

SEQ ID NO: 96 - protein, 4 aa, synthetic construct
  REGION 1..4: note = Description of Artificial Sequence: Synthetic peptide
GPSV

SEQ ID NO: 97 - protein, 16 aa, synthetic construct
  REGION 1..16: note = Description of Artificial Sequence: Synthetic peptide
VECPPCPAPPVAGPSV

SEQ ID NO: 98 - protein, 4 aa, synthetic construct
  REGION 1..4: note = Description of Artificial Sequence: Synthetic peptide
ESKY

SEQ ID NO: 99 - protein, 5 aa, synthetic construct
  REGION 1..5: note = Description of Artificial Sequence: Synthetic peptide
DKTHT

SEQ ID NO: 100 - protein, 4 aa, synthetic construct
  REGION 1..4: note = Description of Artificial Sequence: Synthetic peptide
CPPC

SEQ ID NO: 101 - protein, 4 aa, synthetic construct
  REGION 1..4: note = Description of Artificial Sequence: Synthetic peptide
CPSC

SEQ ID NO: 102 - protein, 11 aa, synthetic construct
  REGION 1..11: note = Description of Artificial Sequence: Synthetic peptide
CPPCPAPELLG

SEQ ID NO: 103 - protein, 10 aa, synthetic construct
  REGION 1..10: note = Description of Artificial Sequence: Synthetic peptide
CPPCPAPGGG

SEQ ID NO: 104 - protein, 11 aa, synthetic construct
  REGION 1..11: note = Description of Artificial Sequence: Synthetic peptide
CPPCPAPEFLG

SEQ ID NO: 105 - protein, 11 aa, synthetic construct
  REGION 1..11: note = Description of Artificial Sequence: Synthetic peptide
CPPAPPAPPVA

SEQ ID NO: 106 - protein, 10 aa, synthetic construct
  REGION 1..10: note = Description of Artificial Sequence: Synthetic peptide
CPPAPAPPVA

SEQ ID NO: 107 - protein, 20 aa, synthetic construct
  REGION 1..20: note = Description of Artificial Sequence: Synthetic peptide
DKTHTCPPCPAPELLGGPSV

SEQ ID NO: 108 - protein, 6 aa, synthetic construct
  REGION 1..6: note = Description of Artificial Sequence: Synthetic peptide
APELLG

SEQ ID NO: 109 - protein, 10 aa, synthetic construct
  REGION 1..10: note = Description of Artificial Sequence: Synthetic peptide
CPPCPAPPVA

SEQ ID NO: 110 - protein, 6 aa, synthetic construct
  REGION 1..6: note = Description of Artificial Sequence: Synthetic peptide
GGGLLG

SEQ ID NO: 111 - protein, 11 aa, synthetic construct
  REGION 1..11: note = Description of Artificial Sequence: Synthetic peptide
CPSCPAPEFLG

SEQ ID NO: 112 - protein, 236 aa, synthetic construct
  REGION 1..236: note = Description of Artificial Sequence: Synthetic polypeptide
DKKAEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 113 - protein, 232 aa, synthetic construct
  REGION 1..232: note = Description of Artificial Sequence: Synthetic polypeptide
DKTVERKCCVECPPCPAPPVAGPSVFLFPPKPKDTLMISRTPEVTCVVVDVSHEDPEVQFNWYVDGMEVHNAKTKPREEQFNSTFRVVSVLTVVHQDWLNGKEYKCKVSNKGLPAPIEKTISKTKGQPREPQVYTLPPSREEMTKNQVSLTCLVKGFYPSDIAVEWESNGQPENNYKTTPPMLDSDGSFFLYSKLTVDKSRWQQGNVFSCSVMHEALHNHYTQKSLSLSPGK

SEQ ID NO: 114 - protein, 233 aa, synthetic construct
  REGION 1..233: note = Description of Artificial Sequence: Synthetic polypeptide
DKRVESKYGPPCPSCPAPEFLGGPSVFLFPPKPKDTLMISRTPEVTCVVVDVSQEDPEVQFNWYVDGVEVHNAKTKPREEQFNSTYRVVSVLTVLHQDWLNGKEYKCKVSNKGLPSSIEKTISKAKGQPREPQVYTLPPSQEEMTKNQVSLTCLVKGFYPSDIAVEWESNGQPENNYKTTPPVLDSDGSFFLYSRLTVDKSRWQEGNVFSCSVMHEALHNHYTQKSLSLSLGK

SEQ ID NO: 115 - protein, 14 aa, synthetic construct
  REGION 1..14: note = Description of Artificial Sequence: Synthetic peptide
YGPPCPPCPAPPVA

SEQ ID NO: 116 - protein, 216 aa, synthetic construct
  REGION 1..216: note = Description of Artificial Sequence: Synthetic polypeptide
SDTGRPFVEMYSEIPEIIHMTEGRELVIPCRVTSPNITVTLKKFPLDTLIPDGKRIIWDSRKGFIISNATYKEIGLLTCEATVNGHLYKTNYLTHRQTNTIIDVVLSPSHGIELSVGEKLVLNCTARTELNVGIDFNWEYPSSKHQHKKLVNRDLKTQSGSEMKKFLSTLTIDGVTRSDQGLYTCAASSGLMTKKNSTFVRVHEKVECPPCPAPPV

(19) United States
(12) Patent Application Publication
(10) Pub. No.: US 2026/0176327 A1
(43) Pub. Date: Jun. 25, 2026

(54) VEGF TRAPS AND MINI-TRAPS AND METHODS FOR TREATING OCULAR DISORDERS AND CANCER

(71) Applicant: Regeneron Pharmaceuticals, Inc., Tarrytown, NY (US)

(72) Inventors: William OLSON, Yorktown Heights, NY (US); Joel MARTIN, Tarrytown, NY (US); Neil STAHL, Carmel, NY (US); Jee KIM, Ardsley, NY (US)

(73) Assignee: Regeneron Pharmaceuticals, Inc., Tarrytown, NY (US)

(21) Appl. No.: 19/553,557

(22) Filed: Mar. 2, 2026

Related U.S. Application Data
(63) Continuation of application No. 18/482,589, filed on Oct. 6, 2023.
Application No. 18/482,589 is a division of application No. 17/314,503, filed on May 7, 2021, now Pat. No. 12,103,960.
(60) Provisional application No. 63/022,178, filed on May 8, 2020.

(51) Int. Cl. C07K 14/71; A61K 38/00; A61P 27/02

(52) U.S. Cl. CPC C07K 14/71; A61P 27/02; A61K 38/00; C07K 2317/53

(57) ABSTRACT

The present relates to VEGF traps and VEGF mini-traps that include VEGF receptor Ig-like domains, fused to a multimerizing component, which bind to VEGF and block its interaction with the VEGF receptor. Such molecules are useful for treating angiogenic eye disorders (e.g., age-related macular degeneration), cancer and for other undesired angiogenesis.

### CROSS REFERENCE TO RELATED APPLICATIONS

This patent application is a Continuation of U.S. application Ser. No. 18/482,589, filed Oct. 6, 2023, which is a Divisional Application of U.S. application Ser. No. 17/314,503, filed on May 7, 2021, which claims priority to U.S. Provisional Application No. 63/022,178, filed May 8, 2020, the disclosures of which are each herein incorporated by reference in their entirety.

### SEQUENCE LISTING

The sequence listing of the present application is submitted electronically as an XML formatted sequence listing with a file name 250298_000525_SL.xml, creation date of Nov. 22, 2023, and a size of 169,892 bytes. This sequence listing submitted is part of the specification and is herein incorporated by reference in its entirety.

### FIELD OF THE INVENTION

The field of the present invention relates to VEGF trap and mini-trap molecules that are useful for treating cancer and angiogenic eye disorders as well as such treatment methods themselves.

### BACKGROUND OF THE INVENTION

Several eye disorders are associated with pathological angiogenesis. For example, the development of age-related macular degeneration (AMD) is associated with a process called choroidal neovascularization (CNV). Leakage from the CNV causes macular edema and collection of fluid beneath the macula resulting in vision loss. Diabetic macular edema (DME) is another eye disorder with an angiogenic component. DME is the most prevalent cause of moderate vision loss in patients with diabetes and is a common complication of diabetic retinopathy, a disease affecting the blood vessels of the retina. Clinically significant DME occurs when fluid leaks into the center of the macula, the light-sensitive part of the retina responsible for sharp, direct vision. Fluid in the macula can cause severe vision loss or blindness. Yet another eye disorder associated with abnormal angiogenesis is central retinal vein occlusion (CRVO). CRVO is caused by obstruction of the central retinal vein that leads to a back-up of blood and fluid in the retina. The retina can also become ischemic, resulting in the growth of new, inappropriate blood vessels that can cause further vision loss and more serious complications. Release of vascular endothelial growth factor (VEGF) contributes to increased vascular permeability in the eye and inappropriate new vessel growth. Thus, inhibiting the angiogenic-promoting properties of VEGF is an effective strategy for treating angiogenic eye disorders.

Various VEGF inhibitors, such as the VEGF trap, Eylea (aflibercept), have been approved to treat such eye disorders. Fragments of VEGF traps, mini-traps, are also useful means by which to treat such angiogenic eye disorders. VEGF mini-traps, however, can suffer from the drawback of reactivity with anti-hinge antibodies (AHA) in the body of a patient. Such AHAs recognize the neoepitope created when C-terminal residues of an immunoglobulin Fc that has been truncated become exposed. Though AHAs have been identified in individuals with autoimmune diseases such as rheumatoid arthritis, the function of such antibodies is not well understood. Falkenburg et al., J. Immunol. 198:82-93 (2017).

### SUMMARY OF THE INVENTION

The present invention provides a VEGF mini-trap characterized by the domain structure:

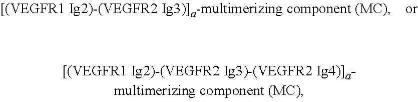

wherein a=1, 2, 3, 4, 5, 6, 7, 8, 9 or 10; and
wherein the MC is a hinge region which is
an IgG1 hinge region comprising a stealth mutation (e.g., wherein residues ELLG (SEQ ID NO: 90) are mutated to PVA);
an IgG1 hinge region-IgG2 hinge region chimera (e.g., wherein the IgG1 residues ELLG (SEQ ID NO: 90) are mutated to PVA);
an IgG1 hinge region comprising an uber mutation (e.g., wherein residues ELLG (SEQ ID NO: 90) are mutated to GGG)
an IgG1 hinge region with residues APEL (SEQ ID NO: 91) mutated (e.g., mutated to GGGF (SEQ ID NO: 92), GGGG (SEQ ID NO: 93) or GGGL (SEQ ID NO: 94));
an IgG1 hinge with an APE residue deletion;
an IgG2 hinge region (e.g., with two or four cysteines which can form disulfide bridges);
an IgG3 hinge region;
an IgG4 hinge region;
an IgG4 hinge region comprising a stealth mutation (e.g., wherein IgG4 hinge residues EFLG (SEQ ID NO: 95) are mutated to PVA); or
an IgG4 hinge region comprising an uber mutation (e.g., wherein residues EFLG (SEQ ID NO: 95) are mutated to GGG)
   e.g., wherein the hinge region comprises, consists of or consists essentially of the amino acid sequence:

(SEQ ID NO: 2)
DKTHTCPPCPAPPVA
(see VEGF mini-trap hIgG1 stealth Fc);
(SEQ ID NO: 3)
DKTHTCPPCPAPGGG
(see VEGF mini-trap hIgG1 uber Fc);
(SEQ ID NO: 4)
ERKCCVECPPCPAPPVA
(see VEGF mini-trap hIgG2 Fc w/4 Cys);
(SEQ ID NO: 5)
VECPPCPAPPVA
(see VEGF mini-trap hIgG2 Fc w/2 Cys);
(SEQ ID NO: 6 or amino acids 6-21 thereof)
EPKSCDTPPPCPPCPAPELLG
(see VEGF mini-trap hIgG3 Fc);
(SEQ ID NO: 7 or amino acids 4-18 thereof)
ESKYGPPCPPCPAPEFLG
(see VEGF mini-trap hIgG4 Fc);
(SEQ ID NO: 8 or amino acids 4-17 thereof)
ESKYGPPCPPCPAPPVA
(see VEGF mini-trap hIgG4 stealth Fc);
(SEQ ID NO: 9 or amino acids 4-17 thereof)
ESKYGPPCPPCPAPGGG
(see VEGF mini-trap hIgG4 uber Fc);
(SEQ ID NO: 10)
DKTHTCPPCPAPPVA
(see VEGF mini-trap hIgG1-2 Chimera Fc);
(SEQ ID NO: 11)
DKTHTCPPCPGGGFLG
(see VEGF mini-trap hIgG1 Mut GGGF (SEQ ID NO: 92)
Fc);
(SEQ ID NO: 12)
DKTHTCPPCPGGGGLG
(see VEGF mini-trap hIgG1 Mut GGGG (SEQ ID NO: 93)
Fc);
(SEQ ID NO: 13)
DKTHTCPPCPGGGLLG
(see VEGF mini-trap hIgG1 Mut GGGL (SEQ ID NO: 94)
Fc);
(SEQ ID NO: 14)
DKTHTCPPCPLLG
(see VEGF mini-trap hIgG1 Del (APE) Fc);
or
(SEQ ID NO: 68)
DKTHTCPLCPAPELLG;

or
any of SEQ ID Nos: 15-22 or 74-77 up to, but not including, the GPSV (SEQ ID NO: 96) IdeS cleavage site as set forth in FIG. 2.

The present invent also provides a VEGF trap characterized by the domain structure

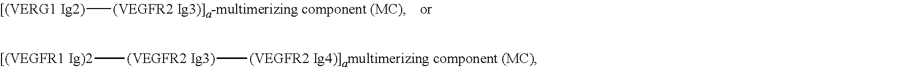

wherein a=1, 2, 3, 4, 5, 6, 7, 8, 9 or 10; and
wherein the MC includes a hinge region and a protease cleavage site which is
an IgG1 hinge region comprising a stealth mutation;
an IgG1 hinge region-IgG2 hinge region chimera;
an IgG1 hinge region with residues APEL (SEQ ID NO: 91) mutated (e.g., to GGGF (SEQ ID NO: 92), GGGG (SEQ ID NO: 93) Or GGGL (SEQ ID NO: 94));
an IgG1 hinge region with APE residues deleted;
an IgG2 hinge region (e.g., including two or four cysteine residues that may form disulfide bridges);
an IgG3 hinge region;
an IgG4 hinge region; or
an IgG4 hinge region comprising a stealth mutation;
   e.g., wherein the MC includes the amino acid sequence:

(SEQ ID NO: 16, or a variant thereof)
EPKSCDKTHTCPPCPAPPVAGPSVFLFPPKPKDT
or amino acids 6-24 thereof
(see VEGF trap hIgG1 stealth Fc);
(SEQ ID NO: 17, or a variant thereof)
EPKSCDKTHTCPPCPAPGGGGPSVFLFPPKPKDT
or amino acids 6-24 thereof
(see VEGF trap hIgG1 uber Fc);
(SEQ ID NO: 18, or a variant thereof)
ERKCCVECPPCPAPPVAGPSVFLFPPKPKDT
or amino acids 1-21 or 6-21 (VECPPCPAPPVAGPSV
(SEQ ID NO: 97)) thereof (see VEGF trap hIgG2
Fcw/2Cys or w/4Cys);
(SEQ ID NO: 19, or a variant thereof)
EPKSCDTPPPCPPCPAPELLGGPSVFLFPPKPKDT
or amino acids 6-25 thereof (see VEGF trap
hIgG3 Fc);
(SEQ ID NO: 20, or a variant thereof)
ESKYGPPCPPCPAPEFLGGPSVFLFPPKPKDT
or amino acids 1-22 thereof (see VEGF trap
hIgG4 Fc);
(SEQ ID NO: 21, or a variant thereof)
ESKYGPPCPPCPAPPVAGPSVFLFPPKPKDT
or amino acids 1-21 thereof (see VEGF trap hIgG4
stealth Fc);
(SEQ ID NO: 22, or a variant thereof)
ESKYGPPCPPCPAPGGGGPSVFLFPPKPKDT
or amino acids 1-21 thereof (see VEGF trap hIgG4
uber Fc);
(SEQ ID NO: 70)
DKTHTCPPCPAPPVAGPSV
(see VEGF trap hIgG1 stealth Fc or hIgG1-2
chimera Fc);
(amino acids 6-25 of SEQ ID NO: 74)
DKTHTCPPCPGGGFLGGPSV
(see VEGF trap hIgG1 mut GGGF (SEQ ID NO: 92) Fc);
(amino acids 6-25 of SEQ ID NO: 75)
DKTHTCPPCPGGGGLGGPSV
(see VEGF trap hIgG1 mut GGGG (SEQ ID NO: 93) Fc);
(amino acids 6-25 of SEQ ID NO: 76)
DKTHTCPPCPGGGLLGGPSV
(see VEGF trap hIgG1 mut GGGL (SEQ ID NO: 94) Fc);
or
(amino acids 6-22 of SEQ ID NO: 77)
DKTHTCPPCPLLGGPSV
(see VEGF trap hIgG1 Del (APE) Fc).

In an embodiment of the invention, any of the VEGF traps or mini-traps set forth herein are characterized by any one or more of the following:
Binds to human VEGF₁₆₅ with a K_{D} of about 1-2 pM or a greater affinity at 25° C., wherein said affinity was measured by surface plasmon resonance;
Has a homodimer molecular weight of less than 97 kDa (e.g., about 50 kDa, 49 kDa, 48 kDa, 47 kDa or 46 kDa; or less than any of the foregoing weights);
Has an IC₅₀ of about 1-2×10⁻¹¹ M for blocking VEGF₁₂₁ and/or VEGF₁₆₅ activation of VEGFR1 and/or VEGFR2 as measured in HEK293 cells expressing the VEGF receptor extracellular domain fused to the intracellular domain of IL18Ralpha or IL18R beta and having an NFkappaB-luciferase-IRES-eGFP reporter gene;
Exhibits less immunogenicity in monkey naïve serum samples and/or human AMD/DME baseline serum samples than that of REGN7483 (SDTGRPFVEMYSEIPEIIHMTEGRELVIPCRVTSPNITVTLKKFPLDTLIPDGKRIIWDSRKGFIISNATY KEIGLLTCEATVNGHLYKTNYLTHRQTNTIIDVVLSPSHGIELSVGEKLVLNCTARTELNVGIDENWEYPS SKHQHKKLVNRDLKTQSGSEMKKFLSTLTIDGVTRSDQGLYTCAASSGLMTKKNSTFVRVHEKDKTHTCPP CPAPELLG (SEQ ID NO: 67)) as measured by anti-drug antibody (ADA) assay;
Forms a 1:1 complex with VEGF₁₆₅ homodimer;
Forms a 1:1:2 complex with VEGF₁₆₅ homodimer and an anti-VEGF Fab fragment (VEGF mini-trap:VEGF₁₆₅ homodimer:Fab fragment);
Reduces retinal neovascularization when dosed intravitreally in an Oxygen Induced Retinopathy (OIR) mouse model;
Exhibits a shorter intravitreal half-live than aflibercept in rabbit eyes when intravitreally injected;
Exhibits lower plasma levels of free molecule when injected intravitreally at 5.5 mg/eye, than aflibercept injected intravitreally at 4 mg/eye in a non-human primate;
Exhibits about 0.1 or 0.2 or 0.1-0.2 micrograms/ml (or less) concentration of free molecule in the plasma of a non-human primate about 336 hours after intravitreal injection of about 5.5 mg/eye;
Does not significantly accumulate in the eye of a non-human primate after four intravitreal injections about every 28 days of about 5.5 mg/eye.
In a composition comprising VEGF traps and/or VEGF mini-trap, such molecules are glycosylated with one or more fucose residues (e.g., about 43-45%), galactose residues (e.g., about 64-72%), sialic acid residues (e.g., about 20-27%), high mannose residues (e.g., Man5) (e.g., about 19-25%) and/or bisecting glycans (e.g., about 1.6-1.9%; i.e., N-glycan having an N-acetyl glucosamine between two regular branches, see e.g., FIG. 26(A)).
One or more histidines are oxidized to 2-oxo-histidine.
One or more tryptophans are deoxidated.
One or more asparagines thereof are glycosylated.
One more serines or threonines are O-glycosylated;
One or more asparagines are deamidated.
One or more Aspartate-Glycine motifs are converted to iso-aspartate-glycine and/or Asn-Gly.
One or more methionines are oxidized.
One or more tryptophans are converted to N-formylkynurenin.
One or more arginines are converted to Arg 3-deoxyglucosone.
The C-terminal residue is not present.
There are one or more non-glycosylated glycosites.
Is xylosylated.
Is glycated at a lysine.
Comprises a cystine with a free-thiol group.
Comprises an intrachain disulfide bridge.
Comprises disulfide bridges in parallel or crossed orientation; and/or
Comprises a lysine or arginine which is carboxymethylated.

In an embodiment of the invention, the MC of any of the VEGF traps comprise the amino acid sequence:

(SEQ ID NO: 23)
GPSVFLFPPKPKDTLMISRTPEVTCVVVDVSHEDPEVQFNWYVDGVEVHN
AKTKPREEQFNSTFRVVSVLTVVHQDWLNGKEYKCKVSNKGLPAPIEKTI
SKTKGQPREPQVYTLPPSREEMTKNQVSLTCLVKGFYPSDIAVEWESNGQ
PENNYKTTPPMLDSDGSFFLYSKLTVDKSRWQQGNVFSCSVMHEALHNHY
TQKSLSLSPGK;
(SEQ ID NO: 24)
GPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTI
SKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHY
TQKSLSLSPGK;
(SEQ ID NO: 25)
GPSVFLFPPKPKDTLMISRTPEVTCVVVDVSHEDPEVQFNWYVDGVEVHN
AKTKPREEQFNSTFRVVSVLTVVHQDWLNGKEYKCKVSNKGLPAPIEKTI
SKTKGQPREPQVYTLPPSREEMTKNQVSLTCLVKGFYPSDIAVEWESNGQ
PENNYKTTPPMLDSDGSFFLYSKLTVDKSRWQQGNVFSCSVMHEALHNHY
TQKSLSLSPGK;
(SEQ ID NO: 26)
GPSVFLFPPKPKDTLMISRTPEVTCVVVDVSQEDPEVQFNWYVDGVEVHN
AKTKPREEQFNSTYRVVSVLTVLHQDWLNGKEYKCKVSNKGLPSSIEKTI
SKAKGQPREPQVYTLPPSQEEMTKNQVSLTCLVKGFYPSDIAVEWESNGQ
PENNYKTTPPVLDSDGSFFLYSRLTVDKSRWQEGNVFSCSVMHEALHNHY
TQKSLSLSLGK;
(SEQ ID NO: 27)
GPSVFLFPPKPKDTLMISRTPEVTCVVVDVSHEDPEVQFNWYVDGVEVHN
AKTKPREEQFNSTFRVVSVLTVVHQDWLNGKEYKCKVSNKGLPAPIEKTI
SKTKGQPREPQVYTLPPSREEMTKNQVSLTCLVKGFYPSDIAVEWESNGQ
PENNYKTTPPMLDSDGSFFLYSKLTVDKSRWQQGNVFSCSVMHEALHNHY
TQKSLSLSPGK;
(SEQ ID NO: 28)
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHY
TQKSLSLSPGK;
(SEQ ID NO: 29)
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHY
TQKSLSLSPGK;
(SEQ ID NO: 30)
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHY
TQKSLSLSPGK;
(SEQ ID NO: 31)
GPSVFLFPPKPKDTLMISRTPEVTCVVVDVSHEDPEVKFNWYVDGVEVHN
AKTKPREEQYNSTYRVVSVLTVLHQDWLNGKEYKCKVSNKALPAPIEKTI
SKAKGQPREPQVYTLPPSRDELTKNQVSLTCLVKGFYPSDIAVEWESNGQ
PENNYKTTPPVLDSDGSFFLYSKLTVDKSRWQQGNVFSCSVMHEALHNHY
TQKSLSLSPGK;
(SEQ ID NO: 78)
VECPPCPAPPVAGPSVFLFPPKPKDTLMISRTPEVTCVVVDVSHEDPEVQ
FNWYVDGVEVHNAKTKPREEQFNSTFRVVSVLTVVHQDWLNGKEYKCKVS
NKGLPAPIEKTISKTKGQPREPQVYTLPPSREEMTKNQVSLTCLVKGFYP
SDIAVEWESNGQPENNYKTTPPMLDSDGSFFLYSKLTVDKSRWQQGNVFS
CSVMHEALHNHYTQKSLSLSPGK;
(SEQ ID NO: 79)
DKTHTCPPCPAPPVAGPSVFLFPPKPKDTLMISRTPEVTCVVVDVSQEDP
EVQFNWYVDGVEVHNAKTKPREEQFNSTYRVVSVLTVLHQDWLNGKEYKC
KVSNKGLPSSIEKTISKAKGQPREPQVYTLPPSRDELTKNQVSLTCLVKG
FYPSDIAVEWESNGQPENNYKTTPPVLDSDGSFFLYSKLTVDKSRWQQGN
VFSCSVMHEALHNHYTQKSLSLSPGK;
(SEQ ID NO: 80)
ERKCCVECPPCPAPPVAGPSVFLFPPKPKDTLMISRTPEVTCVVVDVSHE
DPEVQFNWYVDGVEVHNAKTKPREEQFNSTFRVVSVLTVVHQDWLNGKEY
KCKVSNKGLPAPIEKTISKTKGQPREPQVYTLPPSREEMTKNQVSLTCLV
KGFYPSDIAVEWESNGQPENNYKTTPPMLDSDGSFFLYSKLTVDKSRWQQ
GNVFSCSVMHEALHNHYTQKSLSLSPGK;
(SEQ ID NO: 81)
ESKYGPPCPPCPAPPVAGPSVFLFPPKPKDTLMISRTPEVTCVVVDVSQE
DPEVQFNWYVDGVEVHNAKTKPREEQFNSTYRVVSVLTVLHQDWLNGKEY
KCKVSNKGLPSSIEKTISKAKGQPREPQVYTLPPSQEEMTKNQVSLTCLV
KGFYPSDIAVEWESNGQPENNYKTTPPVLDSDGSFFLYSRLTVDKSRWQE
GNVFSCSVMHEALHNHYTQKSLSLSLGK;
(SEQ ID NO: 82)
DKTHTCPPCPAPPVAGPSVFLFPPKPKDTLMISRTPEVTCVVVDVSHEDP
EVQFNWYVDGVEVHNAKTKPREEQFNSTFRVVSVLTVVHQDWLNGKEYKC
KVSNKGLPAPIEKTISKTKGQPREPQVYTLPPSREEMTKNQVSLTCLVKG
FYPSDIAVEWESNGQPENNYKTTPPMLDSDGSFFLYSKLTVDKSRWQQGN
VFSCSVMHEALHNHYTQKSLSLSPGK;
(SEQ ID NO: 83)
DKTHTCPPCPGGGFLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK;
(SEQ ID NO: 84)
DKTHTCPPCPGGGGLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK;
(SEQ ID NO: 85)
DKTHTCPPCPGGGLLGGPSVFLFPPKPKDTLMISRTPEVTCVVVDVSHED
PEVKFNWYVDGVEVHNAKTKPREEQYNSTYRVVSVLTVLHQDWLNGKEYK
CKVSNKALPAPIEKTISKAKGQPREPQVYTLPPSRDELTKNQVSLTCLVK
GFYPSDIAVEWESNGQPENNYKTTPPVLDSDGSFFLYSKLTVDKSRWQQG
NVFSCSVMHEALHNHYTQKSLSLSPGK;
or
(SEQ ID NO: 86)
DKTHTCPPCPLLGGPSVFLFPPKPKDTLMISRTPEVTCVVVDVSHEDPEV
KFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFY
PSDIAVEWESNGQPENNYKTTPPVLDSDGSFFLYSKLTVDKSRWQQGNVF
SCSVMHEALHNHYTQKSLSLSPGK.

In an embodiment of the invention, a VEGF mini-trap comprises the amino acid sequence:

(SEQ ID NO: 32)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKELSTLTIDGVTRSDQGLYTCAASSGLMTKKNSTEV
RVHEKERKCCVECPPCPAPPVA;
(SEQ ID NO: 33)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKVECPPCPAPPVA;
(SEQ ID NO: 34)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPAPPVA;
(SEQ ID NO: 35)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKESKYGPPCPPCPAPPVA;
(SEQ ID NO: 36)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPAPPVA;
(SEQ ID NO: 37)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPGGGFLG;
(SEQ ID NO: 38)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPGGGGLG;
(SEQ ID NO: 39)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPGGGLLG;
or
(SEQ ID NO: 40)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPLLG.

In an embodiment of the invention, a VEGF Trap comprises the amino acid sequence:

(SEQ ID NO: 41)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKVECPPCPAPPVAGPSVFLEPPKPKDTLMISRTPEVTCVVVDVSHE
DPEVQFNWYVDGVEVHNAKTKPREEQFNSTFRVVSVLTVVHQDWLNGKEY
KCKVSNKGLPAPIEKTISKTKGQPREPQVYTLPPSREEMTKNQVSLTCLV
KGFYPSDIAVEWESNGQPENNYKTTPPMLDSDGSFFLYSKLTVDKSRWQQ
GNVFSCSVMHEALHNHYTQKSLSLSPGK;
(SEQ ID NO: 42)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPAPPVAGPSVFLEPPKPKDTLMISRTPEVTCVVVDV
SQEDPEVQFNWYVDGVEVHNAKTKPREEQFNSTYRVVSVLTVLHQDWLNG
KEYKCKVSNKGLPSSIEKTISKAKGQPREPQVYTLPPSRDELTKNQVSLT
CLVKGFYPSDIAVEWESNGQPENNYKTTPPVLDSDGSFELYSKLTVDKSR
WQQGNVESCSVMHEALHNHYTQKSLSLSPGK;
(SEQ ID NO: 43)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKERKCCVECPPCPAPPVAGPSVFLEPPKPKDTLMISRTPEVTCVVV
DVSHEDPEVQFNWYVDGVEVHNAKTKPREEQFNSTFRVVSVLTVVHQDWL
NGKEYKCKVSNKGLPAPIEKTISKTKGQPREPQVYTLPPSREEMTKNQVS
LTCLVKGFYPSDIAVEWESNGQPENNYKTTPPMLDSDGSFELYSKLTVDK
SRWQQGNVESCSVMHEALHNHYTQKSLSLSPGK;
(SEQ ID NO: 44)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKESKYGPPCPPCPAPPVAGPSVFLEPPKPKDTLMISRTPEVTCVVV
DVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVS
LTCLVKGFYPSDIAVEWESNGQPENNYKTTPPVLDSDGSFELYSRLTVDK
SRWQEGNVESCSVMHEALHNHYTQKSLSLSLGK;
(SEQ ID NO: 45)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPAPPVAGPSVFLEPPKPKDTLMISRTPEVTCVVVDV
SHEDPEVQFNWYVDGVEVHNAKTKPREEQFNSTFRVVSVLTVVHQDWLNG
KEYKCKVSNKGLPAPIEKTISKTKGQPREPQVYTLPPSREEMTKNQVSLT
CLVKGFYPSDIAVEWESNGQPENNYKTTPPMLDSDGSFELYSKLTVDKSR
WQQGNVESCSVMHEALHNHYTQKSLSLSPGK;
(SEQ ID NO: 46)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPGGGFLGGPSVFLEPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFELYSKLTVDKS
RWQQGNVESCSVMHEALHNHYTQKSLSLSPGK;
(SEQ ID NO: 47)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPGGGGLGGPSVFLEPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFELYSKLTVDKS
RWQQGNVESCSVMHEALHNHYTQKSLSLSPGK;
(SEQ ID NO: 48)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPGGGLLGGPSVFLEPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFELYSKLTVDKS
RWQQGNVESCSVMHEALHNHYTQKSLSLSPGK;
or
(SEQ ID NO: 49)
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDENWEYPSSKHQHKKL
VNRDLKTQSGSEMKKELSTLTIDGVTRSDQGLYTCAASSGLMTKKNSTEV
RVHEKDKTHTCPPCPLLGGPSVFLEPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVESCSVMHEALHNHYTQKSLSLSPGK.

In an embodiment of the invention, the VEGF trap or VEGF mini-trap is glycosylated, e.g., wherein said VEGF trap or VEGF mini-trap comprises N-linked glycans; said VEGF trap or VEGF mini-trap comprises O-linked glycans; said VEGF trap or VEGF mini-trap is sialylated, galactosylated and/or fusosylated at one or more residues; said VEGF trap or VEGF mini-trap comprises N-linked mannose-5 glycans (Man5) at one or more residues; and/or said VEGF trap or VEGF mini-trap comprises CHO cell glycosylation. The scope of the present invention includes a composition comprising a heterogeneous mixture of glycosylated variants of the VEGF trap or VEGF mini-trap set forth herein. For example, in an embodiment of the invention, about 47% of the VEGF traps or VEGF mini-traps are sialylated, about 70% of the VEGF traps or VEGF mini-traps are galactosylated, about 36% of the VEGF traps or VEGF mini-traps are fucosylated and/or about 11% of the VEGF traps or VEGF mini-traps are Man-5 glycosylated.

The present invention also includes a complex comprising the VEGF trap or mini-trap homodimer (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; and/or mREGN10515) and VEGF; e.g., wherein VEGF is human VEGF such as human VEGF₁₆₅ or VEGF₁₂₁; and/or the complex includes an anti-VEGF antibody or Fab fragment thereof, e.g., which includes human VEGF and an anti-VEGF Fab fragment wherein the stoichiometric ratio is 1:1:2 (VEGF mini-trap:human VEGF:Fab); and/or wherein the complex is between VEGF mini-trap and VEGF and the stoichiometric ratio of VEGF mini-trap-to-VEGF is 1:1.

The present invention provides a pharmaceutical formulation (e.g., an aqueous pharmaceutical formulation) comprising a VEGF trap or VEGF mini-trap of the present invention (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; and/or mREGN10515) and a pharmaceutically effective carrier.

The present invention also provides an isolated polynucleotide that encodes a VEGF trap or VEGF mini-trap of the present invention (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; and/or mREGN10515), for example, SEQ ID NO: 71, 72 or 73; as well as vectors that include the polynucleotide.

The present invention also provides a host cell comprising the polynucleotide or vector of the present invention (e.g., a Chinese hamster ovary cell).

The present invention further provides an isolated polypeptide comprising, consisting or consisting essentially an amino acid sequence selected from the group consisting of SEQ ID Nos: 32-49.

The present invention also provides a vessel or injection device (e.g., syringe, a disposable syringe or a pre-filled syringe), e.g., which is sterile, comprising a VEGF trap or VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; and/or mREGN10515) or pharmaceutical formulation thereof of the present invention.

The present invention also provides a method for making a VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; and/or mREGN10515) or pharmaceutical formulation thereof comprising contacting a VEGF trap of the present invention with a protease (e.g., IdeS, e.g., comprising the amino acid sequence of any of SEQ ID NOs: 50-65) that cleaves the MC of said VEGF trap and incubating the VEGF trap and protease under conditions that promotes cleavage, and, optionally, combining the VEGF mini-trap product of the cleavage with a pharmaceutically effective carrier.

The present invention also provides a method for making a VEGF trap or VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; and/or mREGN10515) or pharmaceutical formulation thereof comprising incubating a host cell containing a polynucleotide encoding the VEGF trap or VEGF mini-trap or a vector thereof in a culture medium under conditions that promote expression of the trap or mini-trap and, optionally, isolating the trap or mini-trap from the host cell and/or medium and, optionally, combining the VEGF trap or VEGF mini-trap product of the cleavage with a pharmaceutically effective carrier. Optionally, the method includes the step of contacting the VEGF trap with a protease that cleaves the MC of said VEGF trap and incubating the VEGF trap and protease under conditions that promotes cleavage. A VEGF trap or VEGF mini-trap that is the product of such a method also forms part of the present invention.

The present invention also provides a method for treating or preventing an angiogenic eye disorder (e.g., age-related macular degeneration, wet age-related macular degeneration, dry age-related macular degeneration, macular edema, macular edema following retinal vein occlusion, retinal vein occlusion (RVO), central retinal vein occlusion (CRVO), branch retinal vein occlusion (BRVO), retinopathy of prematurity (ROP), diabetic macular edema (DME), choroidal neovascularization (CNV), iris neovascularization, neovascular glaucoma, post-surgical fibrosis in glaucoma, proliferative vitreoretinopathy (PVR), optic disc neovascularization, corneal neovascularization, retinal neovascularization, vitreal neovascularization, pannus, pterygium, vascular retinopathy, diabetic retinopathy in a subject with diabetic macular edema; diabetic retinopathy, non-proliferative diabetic retinopathy, and/or proliferative diabetic retinopathy), in a subject (e.g., a human), comprising administering (e.g., intraocularly or intravitreally) a therapeutically effective amount of VEGF trap or VEGF mini-trap of the present invention (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; and/or mREGN10515) to the subject. In an embodiment of the invention, neovascularization of the retina is reduced in the subject's eye and/or vascular permeability of the retina is reduced in the subject's eye.

The present invention also provides a method for treating or preventing cancer (e.g., lung cancer, breast cancer, colorectal cancer, metastatic colorectal cancer, prostate cancer, skin cancer or stomach cancer), in a subject (e.g., a human), comprising administering (e.g., intravenously) a therapeutically effective amount of VEGF trap or VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; and/or mREGN10515) to the subject.

The present invention also provides a method for administering a VEGF trap or VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; and/or mREGN10515) or pharmaceutical formulation thereof of the present invention, to a subject (e.g., a human), comprising introducing (e.g., by injection, e.g., intravitreal injection) the VEGF trap or VEGF mini-trap or pharmaceutical formulation into the body of the subject.

The present invention also includes a method for reducing neovascularization of the retina and/or vascular permeability of the retina of a subject comprising administering a therapeutically effective amount of VEGF trap or VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; and/or mREGN10515) or pharmaceutical formulation thereof as set forth herein to the subject.

The present invention encompasses any of the VEGF traps or mini-traps consisting of an amino acid sequence set forth herein except further including 1, 2, 3, 4, 5, 6, 7, 8, 9 or 10 (e.g., up to 10) additional amino acids, e.g., at the N-terminal or C-terminal end; as well as any of the related methods of use thereof.

### BRIEF DESCRIPTION OF THE FIGURES

FIG. 1. Summary of VEGF traps and mini-traps with IgG hinge amino acids shown [//=IdeS cleavage site]. VEGF mini-trap from IdeS cleavage refers to the mini-trap generated by IdeS cleavage of the indicated VEGF trap. IgGs includes information concerning the form of IgG in the multimerizing component of the trap or mini-trap.

FIG. 2. VEGF Trap hinge region sequences with IdeS cleavage sites indicated. The location of residues in the Fc hinge region of an IgG1 (PEL and T) that may be reactive with pre-existing anti-hinge antibodies in the serum of a patient are indicated with arrows. Lower, core and upper hinge region residues are indicated with box. (SEQ ID Nos: 15-22, 16, 74-77, respectively)

FIG. 3 (A-C). Summary of VEGF traps and mini-traps with common and descriptive names and molecular weights indicated.

FIG. 4. Schematic representation of typical VEGF mini-trap formation resulting from IdeS cleavage of VEGF trap.

FIG. 5 (A-B). Sequence comparison between aflibercept (REGN3), VEGF mini-trap derived from REGN3 (REGN7483^{F}), VEGF trap hIgG1 stealth Fc, VEGF trap hIgG2 Fc with 4 cys, VEGF mini-trap hIgG2 with 4 cys, VEGF trap hIgG2 Fc with 2 cys, and VEGF mini-trap hIgG2 with 2 cys.

FIG. 6 (A-B). Sequence comparison between aflibercept (REGN3), VEGF mini-trap derived from REGN3 (REGN7483^{F}), REGN10513, REGN10514, REGN10512, REGN10515, REGN10104, REGN10511, REGN10102, REGN10117, and REGN10187. Figure discloses “GGGF”, “GGGG”, and “GGGL” as SEQ ID NOS 92-94, respectively.

FIG. 7 (A-J). Amino acid sequences of VEGF traps and VEGF mini-traps (A) REGN3, REGN7483, (B) REGN10104, mREGN10104, (C) REGN10102, REGN10105, (D) REGN1017, REGN10103, (E) REGN10187, mREGN10187, (F) REGN10511, mREGN10511, (G) REGN10512, mREGN10512, (H) REGN10513, mREGN10513, (I) REGN10514, REGN11095, and (J) REGN10515, mREGN10515. IdeS protease cleavage sites in VEGF traps are indicated by an arrow. N-terminal VEGFR1 domain 2 is dark shaded and VEGFR2 domain 3 is light shaded and boxed. C-terminal Fc domain in VEGF traps are dark shaded. Hinge regions of VEGF traps and VEGF mini-traps are unshaded. Figure discloses “GGGF”, “GGGG”, and “GGGL” as SEQ ID NOS 92-94, respectively.

FIG. 8 (A-E). Bioassay activity of VEGF traps and VEGF mini-traps (REGN10117 (A), REGN10104 and mREGN10104 (B), REGN10102 (C), REGN10187 and mREGN10187 (D); and REGN10103 and REGN10105 (E); along with controls REGN7483, REGN3 (VEGFT) and VEGF₁₂₁ in each) blocking VEGF₁₂₁ activation (VEGF₁₂₁ at 40 pM) of VEGFR1 expressing cells (293/D9/VEGFR1).

FIG. 9 (A-E). Bioassay activity of VEGF traps and VEGF mini-traps (REGN10102 (A), REGN10104 and mREGN10104 (B) REGN10117 (C), REGN10187 (D) REGN10187 and mREGN10187 (D); and REGN10103 and REGN101005 (E); along with controls REGN7483, REGN3 (VEGFT) and VEGF₁₂₁ in each) blocking VEGF₁₂₁ activation (VEGF₁₂₁ at 60 pM) of VEGFR2 expressing cells (293/D9/VEGFR2).

FIG. 10 (A-F). Non-reducing SDS-PAGE analysis of Ides protease-mediated cleavage of various VEGF Traps (REGN3 (A), REGN10102 (B), REGN10511 (C), REGN10512 (D), REGN10514 (E) and REGN10515 (F)) over time (after incubation for 0, 1, 2 or 4 hours or overnight (O/N)). Figure discloses “GGGF” and “GGGL” as SEQ ID NOS 92 and 94, respectively.

FIG. 11 (A-B). Anti-drug antibody electrochemiluminescent immunoassay (ECL)—Bridging Format. (A) diagram of assay format. (B) Assay of REGN7483F, REGN10105 (from IdeS cleaved VEGF trap with IgG2 Fc with 2 Cys), mREGN10104 (IdeS cleaved from REGN10104), REGN10105 (recombinantly expressed in CHO cells), REGN10103 (from IdeS cleaved from VEGF trap with IgG2 Fc with 4 Cys) and mREGN10187 for pre-existing anti-hinge antibodies (AHA) responses in naïve monkey serum samples.

FIG. 12. Anti-drug antibody electrochemiluminescent immunoassay (ECL)-Bridging Format. Assay of REGN7483F, REGN10105 (from IdeS cleaved VEGF trap with IgG2 Fc with 2 Cys), mREGN10104 (IdeS cleaved from REGN10104), REGN10105 (recombinantly expressed in CHO cells), REGN10103 (IdeS cleaved from VEGF trap with IgG2 Fc with 4 Cys) and mREGN10187 for pre-existing anti-hinge antibodies (AHA) responses in human AMD/DME serum samples.

FIG. 13. Schematic diagram of human IgG4 stealth and human IgG1 stealth for reduction of effector functions. Figure discloses “CPPC” and “EFLG” as SEQ ID NOS 100 and 95, respectively.

FIG. 14. Schematic diagram of IgG4 antibody possessing stealth mutations (SEQ ID NOS 111 and 109, respectively, in order of appearance).

FIG. 15. Sequence of IgG4 with IgG2 lower hinge region sequence. Sequence of construct is boxed. IgG4/2 hybrid VEGF traps possessing an MC with the IgG2 boxed hinge sequence as shown in the figure (or starting at the IgG4 ESKY (SEQ ID NO: 98) motif) and mini-traps thereof (cleaved before the GPSV (SEQ ID NO: 96)) are part of the present invention. Figure discloses SEQ ID NOS 112-114, respectively, in order of appearance.

FIG. 16. Schematic diagram of IgG1 antibody possessing stealth mutations (SEQ ID NOS 102 and 109, respectively, in order of appearance).

FIG. 17. Sequence of IgG1 with effectorless CH2 sequence. IgG1/2 hybrid VEGF traps possessing an MC with the IgG2 boxed hinge sequence as shown in the figure (or starting at the IgG1 DKTHT (SEQ ID NO: 99) motif) and mini-traps thereof (cleaved before the GPSV (SEQ ID NO: 96)) are part of the present invention. Figure discloses SEQ ID NOS 112-114, respectively, in order of appearance.

FIG. 18 (A-B). Analysis of (A) REGN10105: hVEGF165 and (B) REGN11095: hVEGF₁₆₅ complexes (solid lines) by size exclusion chromatography coupled to multi-angle laser light scattering (SEC-MALS). Chromatograms from individual samples of REGN10105 or REGN11095 (grey dashed line) and hVEGF₁₆₅ (black dashed line) are also overlaid. Relative refractive index as a function of retention volume is shown for each sample and the measured molar masses of resolved peaks are indicated.

FIG. 19 (A-D). Cryo-EM analyses of structure of VEGF mini-trap REGN10105 in complex with VEGF and REGN18 Fab. Samples of hVEGF alone, REGN10105 alone, REGN10105 with hVEGF or REGN10105, hVEGF and REGN18 Fab were analyzed. (A) SEC-MALS chromatogram of REGN10105/VEGF/REGN18 Fab mixture. (B) raw Cryo-EM image, complexes/particles circled. (C) selection of 2D class averages; arrows indicate VEGFR2 domain 3. (D) individual 2D classes; arrow indicates VEGF or VEGFR2 domain 3.

FIG. 20 (A-B). Summary of (A) avascular and (B) abnormal areas observed in retinas from oxygen-induced retinopathy mouse models intravitreally treated with human Fc, aflibercept, REGN11095 or REGN10105. Retinal flat mounts stained with GS Lectin I (for avascular area study) or NG2 (for abnormal area study) were analyzed.

FIG. 21. Summary of normalized (to hFc treated) abnormal areas observed in retinas from oxygen-induced retinopathy mouse models uninjected, or intravitreally treated with human Fc, aflibercept (0.05, 0.5 or 5 micrograms) or REGN10105 (0.025, 0.25 or 2.5 micrograms). Retinal flat mounts stained with or NG2 were analyzed. Data were normalized to the Fc treated group.

FIG. 22 (A-B). Summary of (A) avascular and (B) abnormal areas (normalized to hFc treated) observed in retinas from oxygen-induced retinopathy mouse models systemically administered human Fc, aflibercept, REGN11095 or REGN10105. Retinal flat mounts stained with GS Lectin I (for avascular area study) or NG2 (for abnormal area study) were analyzed.

FIG. 23 (A-F). Decay analysis of VEGF traps in vitreous of New Zealand White rabbits. (A) REGN3 (animals 428, 429 and 430), (B) REGN7483 (animals 434, 435 and 436), (C) REGN3 (animals 570, 571 and 572), (D) REGN10105 (animals 573, 574 and 575) and (E) REGN11095 (animals 576, 577 and 578). OS=oculus sinister, OD=oculus dexter. (F) intraocular pressure (IOP) (mmHg) before and after IVT injection of REGN3, REGN10105 or REGN11095.

FIG. 24 (A-D). Analyses of free and bound VEGF mini-traps in eyes of male and female non-human primates, over time, that have been dosed multiple times (on days 1, 29, 57 and 85) with REGN10105 or REGN11095. (A) free REGN10105, (B) free REGN11095, (C) bound REGN10105, (D) bound REGN11095.

FIG. 25. Systemic (plasma) VEGF trap and mini-trap concentrations, over time, in non-human primates dosed intravitreally with REGN7483 (5.5 mg/eye), REGN10105 (5.5 mg/eye) or aflibercept (4 mg/eye).

FIG. 26 (A-B). HILIC-FLR Chromatogram of N-linked Glycans from REGN10103 and REGN10105 (A) overlaid and (B) separated. REGN10103 or REGN10105 with any one or more of the indicated N-glycans or compositions having the glycan profile set forth in the figure are part of the present invention.

### DETAILED DESCRIPTION OF THE INVENTION

The present invention provides VEGF traps and VEGF mini-traps which, relative to other mini-traps, exhibit superior stability and low viscosity—particularly at high concentrations. Such characteristics make the VEGF traps and mini-traps well suited for formulation at a high concentration which, in turn, facilitates high doses in a low volume and, thus, less frequent dosing. The anatomy and physiology of the eye makes it undesirable to inject a large volume intravitreally; thus, intravitreal injections should be done at a volume of no more than about 100 microliters. This combination of factors makes development of protein-based drugs, useful for intravitreal injection of a sufficient amount in a suitable volume, technically challenging. Maximizing the amount of drug delivered, so as to minimize the number of doses needed over a given time period, is an even greater challenge. The traps and mini-traps provided herein have overcome these technical hurdles.

The viscosity of aqueous compositions including VEGF mini-traps set forth herein, such as REGN10105, is low even relative to other VEGF mini-traps. The low viscosity allows intravitreal administration with higher gauge needles which minimizes patient discomfort and increases patient compliance.

Moreover, the VEGF mini-traps set forth herein, e.g., REGN10105 and REGN11095, exhibit greater stability (lower formation of high molecular weight species) than aflibercept under accelerated conditions. REGN10105 is also particularly photostability relative to other VEGF mini-traps such as REGN7483.

The level of potential systemic exposure following intravitreal administration of a VEGF mini-trap set forth herein, e.g., REGN10105, is also lower than that of aflibercept. Specifically, the half-life in the rabbit vitreous of REGN10105 is shorter leading to less likelihood of clearance from the eye into the rest of the body.

The VEGF mini-traps set forth herein, characterized, in vitro, as having less reactivity with pre-existing anti-hinge antibodies, have less potential for immunogenic reactions, when administered. For example, a VEGF mini-trap with an IgG2-based Fc hinge region lacks the PEL and T residues in IgG1-based Fc hinge regions (such as in REGN7483) that may be reactive with pre-existing anti-hinge antibodies.

VEGF Traps are VEGF receptor-based chimeric molecules which include two or more immunoglobulin (Ig)-like domains of a VEGF receptor such as VEGFR1 (also referred to as Flt1) (e.g., Ig2 domain of VEGFR1 (VEGFR1d2)) and/or VEGFR2 (also referred to as Flk1 or KDR) (e.g., Ig3 domain of VEGFR2 (VEGFR2d3)), preferably arranged as [(VEGFR1d2)-(VEGFR2d3)]ₙ (e.g., wherein n=1) on each polypeptide chain of the VEGF trap, and also contain an Fc domain which facilitates the multimerization (e.g., dimerization) of two or more chimeric polypeptides).

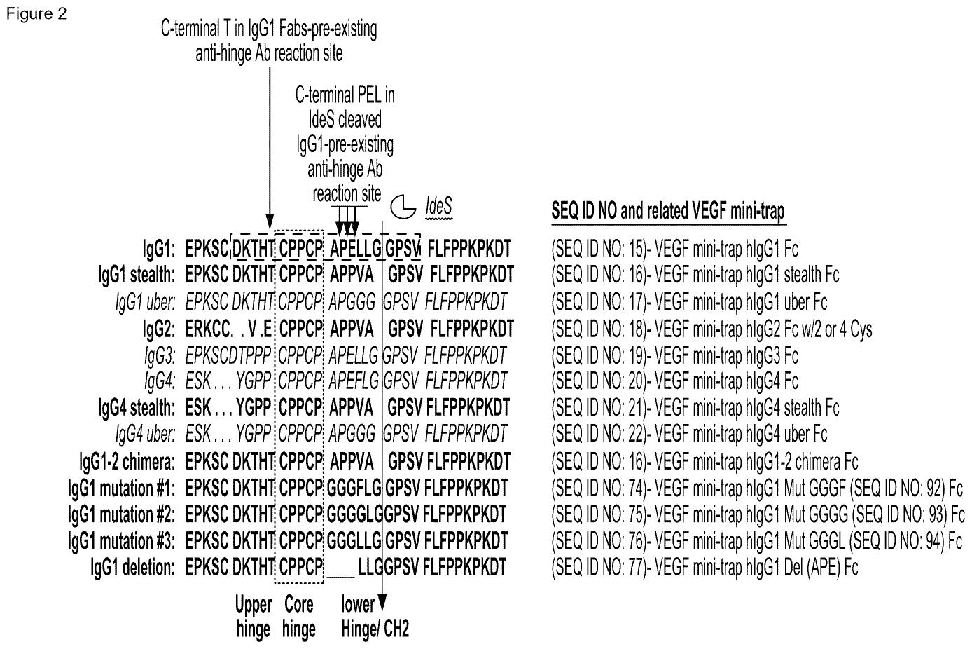

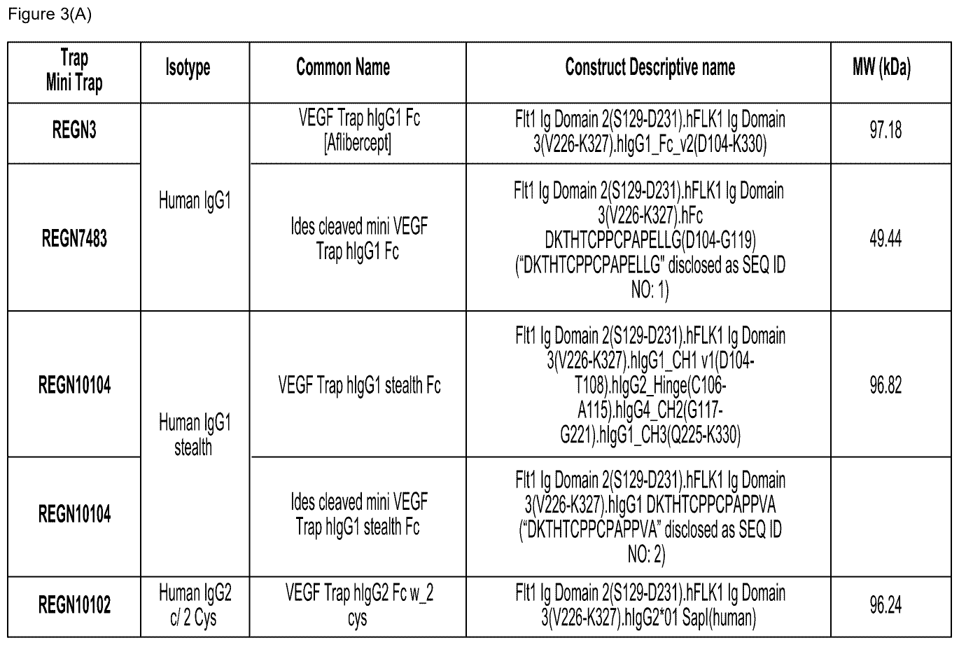

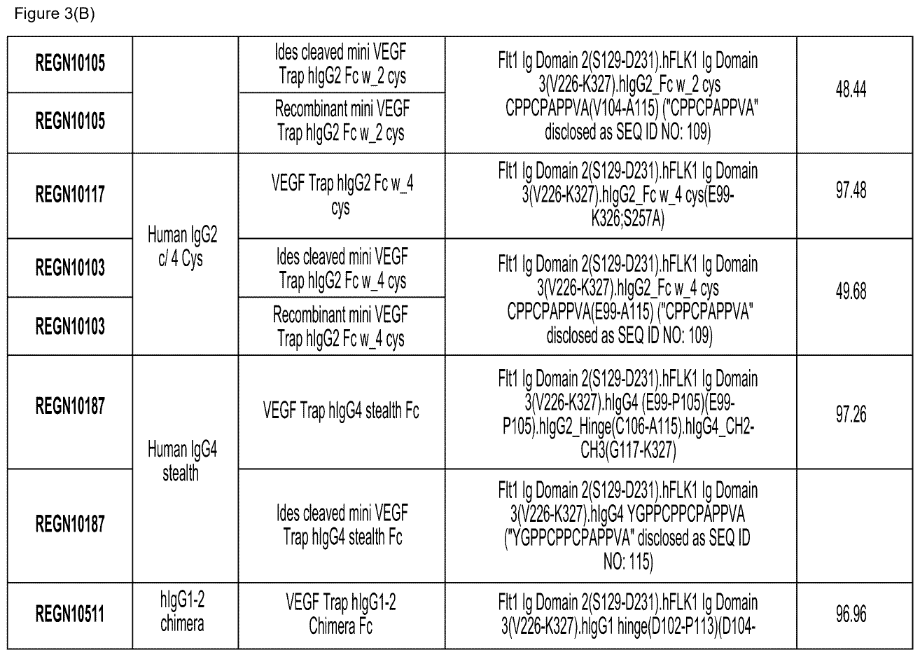

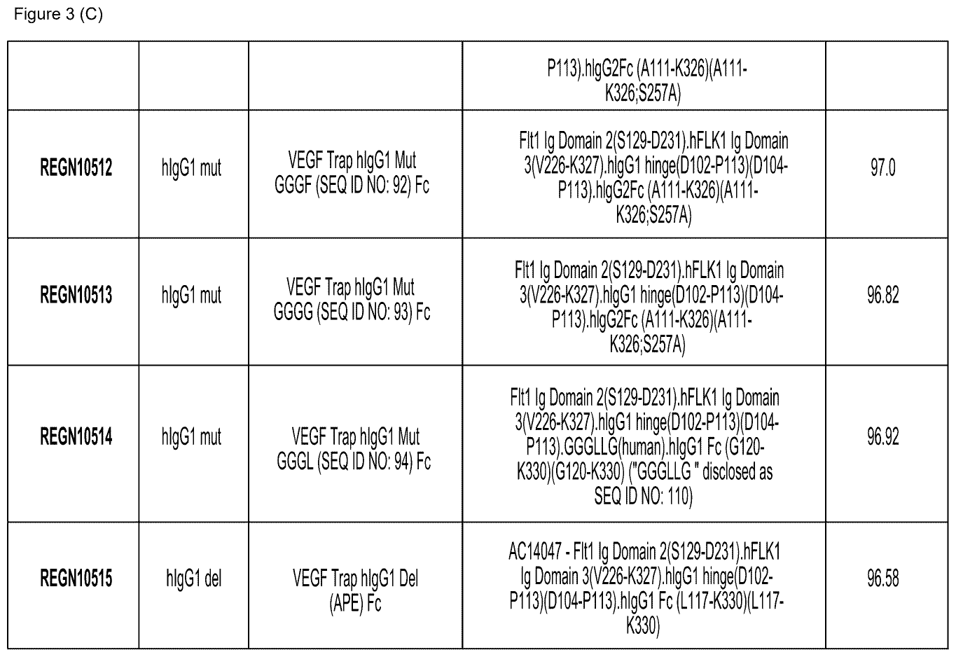

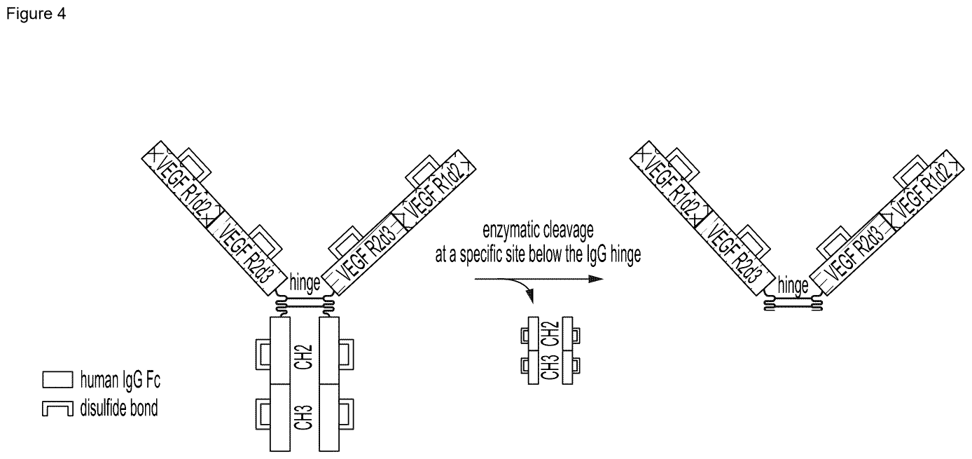

A VEGF mini-trap includes VEGF binding domains (e.g., VEGFR1 Ig domain 2 and VEGFR2 Ig domain 3, arranged in a manner similar to that of VEGF trap such as aflibercept) linked to a fragment of an Fc domain, preferably, the hinge region of the Fc domain (e.g., including the upper hinge region (sometimes beginning with a E-(P, S, or R)-K motif), the core hinge region and the lower hinge region), extending up to, but not including, the GPSV (SEQ ID NO: 96) cleavage site for IdeS protease (see e.g., FIG. 2). The hinge region preferably includes one or more (e.g., 2 or 4) cysteines that can form a cysteine bridge with another VEGF mini-trap polypeptide chain. Preferably, VEGF traps and mini-traps are homodimeric complexes of such polypeptides. VEGF traps and mini-traps may also be in composition including heterogeneous populations of such monomer polypeptides and homodimers. Such monomers, multimers and compositions form part of the present invention. The polypeptide homodimers can be bound together, for example, via disulfide bridges. A schematic of a VEGF trap and a VEGF mini-trap are set forth in FIG. 4.

The “lg” domain of a VEGFR refers to the indicated immunoglobulin-like domain of the indicated VEGF receptor.

Standard methods in molecular biology are described Sambrook, Fritsch and Maniatis (1982 & 1989 2^{nd} Edition, 2001 3^{rd} Edition) Molecular Cloning, A Laboratory Manual, Cold Spring Harbor Laboratory Press, Cold Spring Harbor, N.Y.; Sambrook and Russell (2001) Molecular Cloning, 3^{rd} ed., Cold Spring Harbor Laboratory Press, Cold Spring Harbor, N.Y.; Wu (1993) Recombinant DNA, Vol. 217, Academic Press, San Diego, Calif.). Standard methods also appear in Ausbel, et al. (2001) Current Protocols in Molecular Biology, Vols. 1-4, John Wiley and Sons, Inc. New York, N.Y., which describes cloning in bacterial cells and DNA mutagenesis (Vol. 1), cloning in mammalian cells and yeast (Vol. 2), glycoconjugates and protein expression (Vol. 3), and bioinformatics (Vol. 4).

General methods for protein purification including immunoprecipitation, chromatography, electrophoresis, centrifugation, and crystallization are described (Coligan et al. (2000) Current Protocols in Protein Science, Vol. 1, John Wiley and Sons, Inc., New York). Chemical analysis, chemical modification, post-translational modification, production of fusion proteins, glycosylation of proteins are described (see e.g., Coligan et al. (2000) Current Protocols in Protein Science, Vol. 2, John Wiley and Sons, Inc., New York; Ausubel, et al. (2001) Current Protocols in Molecular Biology, Vol. 3, John Wiley and Sons, Inc., NY, N.Y., pp. 16.0.5-16.22.17; Sigma-Aldrich, Co. (2001) Products for Life Science Research, St. Louis, Mo.; pp. 45-89; Amersham Pharmacia Biotech (2001) BioDirectory, Piscataway, N.J., pp. 384-391). Production, purification, and fragmentation of polyclonal and monoclonal antibodies are described (Coligan et al. (2001) Current Protcols in Immunology, Vol. 1, John Wiley and Sons, Inc., New York; Harlow and Lane (1999) Using Antibodies, Cold Spring Harbor Laboratory Press, Cold Spring Harbor, N.Y.; Harlow and Lane, supra). Standard techniques for characterizing ligand/receptor interactions are available (see, e.g., Coligan et al. (2001) Current Protocols in Immunology, Vol. 4, John Wiley, Inc., New York).

Methods for flow cytometry, including fluorescence activated cell sorting (FACS), are available (see, e.g., Owens et al. (1994) Flow Cytometry Principles for Clinical Laboratory Practice, John Wiley and Sons, Hoboken, N.J.; Givan (2001) Flow Cytometry, 2nd ed.; Wiley-Liss, Hoboken, N.J.; Shapiro (2003) Practical Flow Cytometry, John Wiley and Sons, Hoboken, N.J.). Fluorescent reagents suitable for modifying nucleic acids, including nucleic acid primers and probes, polypeptides, and antibodies, for use, e.g., as diagnostic reagents, are available (Molecular Probes (2003) Catalogue, Molecular Probes, Inc., Eugene, Oreg.; Sigma-Aldrich (2003) Catalogue, St. Louis, Mo.).

Standard methods of histology of the immune system are described (see e.g., Muller-Harmelink (ed.) (1986) Human Thymus: Histopathology and Pathology, Springer Verlag, New York, N.Y.; Hiatt et al. (2000) Color Atlas of Histology, Lippincott, Williams, and Wilkins, Phila, Pa.; Louis et al. (2002) Basic Histology: Text and Atlas, McGraw-Hill, New York, N.Y.).

### VEGF Mini-Traps and VEGF Traps

The VEGF mini-traps of the present invention exhibit superior VEGF neutralizing activity along with low immunogenicity and viscosity and high stability. Moreover, the VEGF Traps of the present invention are well suited for generation of such VEGF mini-traps insofar as they are cleavable with a protease such as IdeS protease (e.g., Streptococcus pyogenes IdeS).

The present invention provides VEGF mini-traps that comprise the following domain structure:

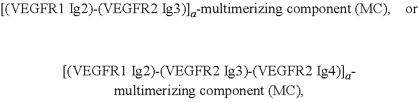

wherein a=1, 2, 3, 4, 5, 6, 7, 8, 9 or 10; and
wherein the MC is a hinge region which is
an IgG1 hinge region comprising a stealth mutation (e.g., wherein residues ELLG (SEQ ID NO: 90) are mutated to PVA);
an IgG1 hinge region comprising an uber mutation (e.g., wherein residues ELLG (SEQ ID NO: 90) are mutated to GGG)
an IgG1 hinge region-IgG2 hinge region chimera (e.g., wherein the IgG1 residues ELLG (SEQ ID NO: 90) are mutated to PVA);
an IgG1 hinge region with residues APEL (SEQ ID NO: 91) mutated (e.g., mutated to GGGF (SEQ ID NO: 92), GGGG (SEQ ID NO: 93) Or GGGL (SEQ ID NO: 94));
an IgG1 hinge with an APE residue deletion;
an IgG2 hinge region (e.g., with two or four cysteines which can form disulfide bridges);
an IgG3 hinge region;
an IgG4 hinge region;
an IgG4 hinge region comprising a stealth mutation (e.g., wherein IgG4 hinge residues EFLG (SEQ ID NO: 95) are mutated to PVA);
an IgG4 hinge region comprising an uber mutation (e.g., wherein residues EFLG (SEQ ID NO: 95) are mutated to GGG)
   or
the hinge region containing product of IdeS proteolytic cleavage of a human Fc;
   and multimers (e.g., homodimers) thereof.

In an embodiment of the invention, the VEGF mini-trap hinge region comprises, or consists essentially of, or consists of the amino acid sequence:

(SEQ ID NO: 2)
DKTHTCPPCPAPPVA
(see VEGF mini-trap hIgG1 stealth Fc);
(SEQ ID NO: 3)
DKTHTCPPCPAPGGG
(see VEGF mini-trap hIgG1 uber Fc);
(SEQ ID NO: 4)
ERKCCVECPPCPAPPVA
(see VEGF mini-trap hIgG2 Fc w/4 Cys);
(SEQ ID NO: 5)
VECPPCPAPPVA
(see VEGF mini-trap hIgG2 Fc w/2 Cys);
(SEQ ID NO: 6 or amino acids 6-21 thereof
(underscored))
EPKSCDTPPPCPPCPAPELLG
(see VEGF mini-trap hIgG3 Fc);
(SEQ ID NO: 7 or amino acids 4-18 thereof
(underscored))
ESKYGPPCPPCPAPEFLG
(see VEGF mini-trap hIgG4 Fc);
(SEQ ID NO: 8 or amino acids 4-17 thereof
(underscored))
ESKYGPPCPPCPAPPVA
(see VEGF mini-trap hIgG4 stealth Fc);
(SEQ ID NO: 9 or amino acids 4-17 thereof
(underscored))
ESKYGPPCPPCPAPGGG
(see VEGF mini-trap hIgG4 uber Fc);
(SEQ ID NO: 10)
DKTHTCPPCPAPPVA
(see VEGF mini-trap hIgG1-2 Chimera Fc);
(SEQ ID NO: 11)
DKTHTCPPCPGGGFLG
(see VEGF mini-trap hIgG1
Mut GGGF (SEQ ID NO: 92) Fc);
(SEQ ID NO: 12)
DKTHTCPPCPGGGGLG
(see VEGF mini-trap hIgG1 Mut
GGGG (SEQ ID NO: 93) Fc);
(SEQ ID NO: 13)
DKTHTCPPCPGGGLLG
(see VEGF mini-trap hIgG1 Mut
GGGL (SEQ ID NO: 94) Fc);
(SEQ ID NO: 14)
DKTHTCPPCPLLG
(see VEGF mini-trap hIgG1 Del (APE) Fc);
or
(SEQ ID NO: 68)
DKTHTCPLCPAPELLG.

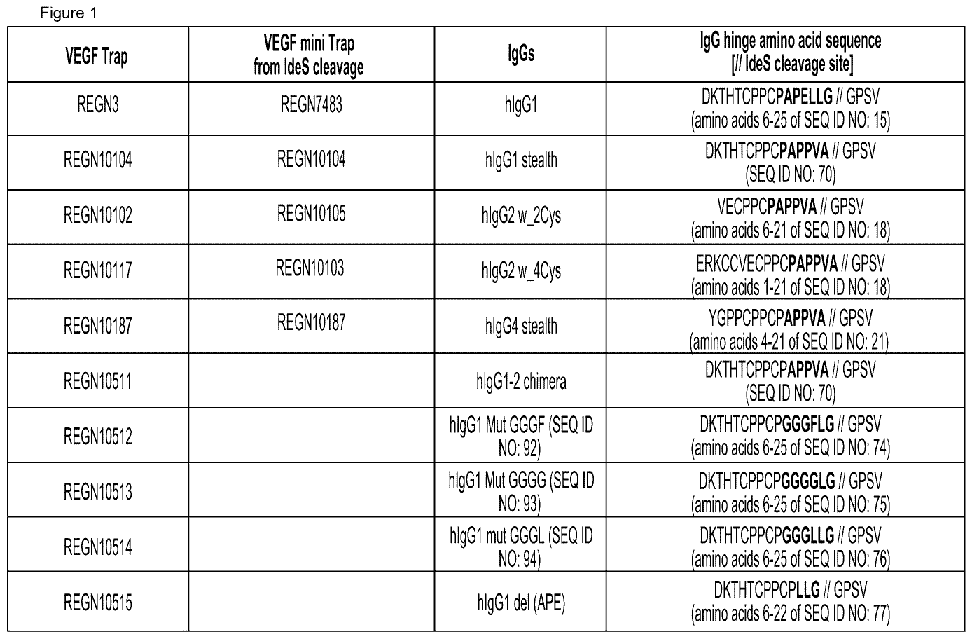

or
the hinge region of any of the MCs set forth herein (e.g., any of SEQ ID NOs: 78-86) or any of those set forth in any of FIG. 1, 2, 3 5, 6, 7 or 13-17.

The IgG hinge region of aflibercept (REGN3) and REGN7483 comprise the amino acid sequence: DKTHTCPPCPAPELLG (SEQ ID NO: 1). In an embodiment of the invention, VEGF mini-traps and VEGF traps defined herein are with the proviso that the hinge region of the MC of aflibercept and/or the hinge region of REGN7483 is/are excluded.

The present invention also includes VEGF traps that comprise the following domain structure:

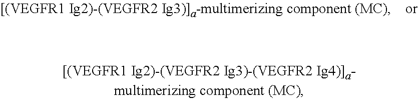

wherein a=1, 2, 3, 4, 5, 6, 7, 8, 9 or 10; and
wherein the MC is an immunoglobulin Fc polypeptide that includes a hinge region and a cleavage site for a protease such as IdeS (but, in an embodiment of the invention, the Fc is not wild-type human IgG1). In an embodiment of the invention the VEGF trap MC is a human Fc that includes:
an IgG1 hinge region comprising a stealth or uber mutation;
an IgG1 hinge region-IgG2 hinge region chimera;
an IgG1 hinge region with residues APEL (SEQ ID NO: 91) mutated (e.g., mutated to GGGF (SEQ ID NO: 92), GGGG (SEQ ID NO: 93) Or GGGL (SEQ ID NO: 94));
an IgG1 APE residue deletion;
an IgG2 hinge region (e.g., with two or four cysteines which can form disulfide bridges);
an IgG3 hinge region;
an IgG4 hinge region;
an IgG4 hinge region comprising a stealth or uber mutation;
   or
any of the MCs set forth herein.

In an embodiment of the invention, the VEGF trap MC includes the amino acid sequence (wherein v is an IdeS protease cleavage site):

(SEQ ID NO: 16, or a variant thereof)
EPKSCDKTHTCPPCPAPPVAGPSVFLFPPKPKDT
or amino acids 6-24 thereof
(see VEGF trap hIgG1 stealth Fc);
(SEQ ID NO: 17, or a variant thereof)
EPKSCDKTHTCPPCPAPGGGGPSVFLFPPKPKDT
or amino acids 6-24 thereof
(see VEGF trap hIgG1 uber Fc);
(SEQ ID NO: 18, or a variant thereof)
ERKCCVECPPCPAPPVAGPSVFLFPPKPKDT
or amino acids 1-21 or 6-21 (VECPPCPAPPVAGPSV
(SEQ ID NO: 97)) thereof (see VEGF trap hIgG2
Fcw/2Cys or w/4Cys);
(SEQ ID NO: 19, or a variant thereof)
EPKSCDTPPPCPPCPAPELLGGPSVFLFPPKPKDT
or amino acids 6-25 thereof (see VEGF trap
hIgG3 Fc);
(SEQ ID NO: 20, or a variant thereof)
ESKYGPPCPPCPAPEFLGGPSVFLFPPKPKDT
or amino acids 1-22 thereof (see VEGF trap
hIgG4 Fc);
(SEQ ID NO: 21, or a variant thereof)
ESKYGPPCPPCPAPPVAGPSVFLFPPKPKDT
or amino acids 1-21 thereof (see VEGF trap hIgG4
stealth Fc);
(SEQ ID NO: 22, or a variant thereof)
ESKYGPPCPPCPAPGGGGPSVFLFPPKPKDT
or amino acids 1-21 thereof (see VEGF trap hIgG4
uber Fc);
(SEQ ID NO: 70)
DKTHTCPPCPAPPVAGPSV
(see VEGF trap hIgG1 stealth Fc or hIgG1-2
chimera Fc);
(amino acids 6-25 of SEQ ID NO: 74)
DKTHTCPPCPGGGFLGGPSV
(see VEGF trap hIgG1 mut GGGF (SEQ ID NO: 92) Fc);
(amino acids 6-25 of SEQ ID NO: 75)
DKTHTCPPCPGGGGLGGPSV
(see VEGF trap hIgG1 mut GGGG (SEQ ID NO: 93) Fc);
(amino acids 6-25 of SEQ ID NO: 76)
DKTHTCPPCPGGGLLGGPSV
(see VEGF trap hIgG1 mut GGGL (SEQ ID NO: 94) Fc);
or
(amino acids 6-22 of SEQ ID NO: 77)
DKTHTCPPCPLLGGPSV
(see VEGF trap hIgG1 Del (APE) Fc).

See e.g., FIG. 2.

In an embodiment of the invention, the VEGF trap MC comprises the amino acid sequence:

(SEQ ID NO: 23)
GPSVFLFPPKPKDTLMISRTPEVTCVVVDVSHEDPEVQFNWYVDGVEVH
NAKTKPREEQFNSTFRVVSVLTVVHQDWLNGKEYKCKVSNKGLPAPIEK
TISKTKGQPREPQVYTLPPSREEMTKNQVSLTCLVKGFYPSDIAVEWES
NGQPENNYKTTPPMLDSDGSFFLYSKLTVDKSRWQQGNVFSCSVMHEAL
HNHYTQKSLSLSPGK;
(SEQ ID NO: 24)
GPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGK;
(SEQ ID NO: 25)
GPSVFLFPPKPKDTLMISRTPEVTCVVVDVSHEDPEVQFNWYVDGVEVH
NAKTKPREEQFNSTFRVVSVLTVVHQDWLNGKEYKCKVSNKGLPAPIEK
TISKTKGQPREPQVYTLPPSREEMTKNQVSLTCLVKGFYPSDIAVEWES
NGQPENNYKTTPPMLDSDGSFFLYSKLTVDKSRWQQGNVFSCSVMHEAL
HNHYTQKSLSLSPGK;
(SEQ ID NO: 26)
GPSVFLFPPKPKDTLMISRTPEVTCVVVDVSQEDPEVQFNWYVDGVEVH
NAKTKPREEQFNSTYRVVSVLTVLHQDWLNGKEYKCKVSNKGLPSSIEK
TISKAKGQPREPQVYTLPPSQEEMTKNQVSLTCLVKGFYPSDIAVEWES
NGQPENNYKTTPPVLDSDGSFFLYSRLTVDKSRWQEGNVFSCSVMHEAL
HNHYTQKSLSLSLGK;
(SEQ ID NO: 27)
GPSVFLFPPKPKDTLMISRTPEVTCVVVDVSHEDPEVQFNWYVDGVEVH
NAKTKPREEQFNSTFRVVSVLTVVHQDWLNGKEYKCKVSNKGLPAPIEK
TISKTKGQPREPQVYTLPPSREEMTKNQVSLTCLVKGFYPSDIAVEWES
NGQPENNYKTTPPMLDSDGSFFLYSKLTVDKSRWQQGNVFSCSVMHEAL
HNHYTQKSLSLSPGK;
(SEQ ID NO: 28)
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGK;
(SEQ ID NO: 29)
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGK;
(SEQ ID NO: 30)
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGK;
or
(SEQ ID NO: 31)
GPSVFLFPPKPKDTLMISRTPEVTCVVVDVSHEDPEVKFNWYVDGVEVH
NAKTKPREEQYNSTYRVVSVLTVLHQDWLNGKEYKCKVSNKALPAPIEK
TISKAKGQPREPQVYTLPPSRDELTKNQVSLTCLVKGFYPSDIAVEWES
NGQPENNYKTTPPVLDSDGSFFLYSKLTVDKSRWQQGNVFSCSVMHEAL
HNHYTQKSLSLSPGK.

In an embodiment of the invention, any of the VEGF mini-traps set forth herein do not include such a MC.

In an embodiment of the invention, a multimerizing component that includes an Fc domain hinge region or variant or fragment thereof (e.g., from IgG4) includes a S108P mutation, e.g., to promote dimer stabilization.

In an embodiment of the invention, a VEGF trap or VEGF mini-trap of the present invention comprises a modified Fc domain or fragment thereof (e.g., wherein the fragment includes the hinge region) having reduced effector function. As used herein, a “modified Fc domain having reduced effector function” means any Fc portion of an immunoglobulin that has been modified, mutated, truncated, etc., relative to a wild-type, naturally occurring Fc domain such that a molecule comprising the modified Fc exhibits a reduction in the severity or extent of at least one effect selected from the group consisting of cell killing (e.g., ADCC and/or CDC), complement activation, phagocytosis and opsonization, relative to a comparator molecule comprising the wild-type, naturally occurring version of the Fc portion. In certain embodiments, a “modified Fc domain having reduced effector function” is an Fc domain with reduced or attenuated binding to an Fc receptor (e.g., FcγR).

In certain embodiments of the present invention, the modified Fc domain is a variant IgG1 Fc or a variant IgG4 Fc comprising a substitution in the hinge region. For example, a modified Fc for use in the context of the present invention may comprise a variant IgG1 Fc wherein at least one amino acid of the IgG1 Fc hinge region is replaced with the corresponding amino acid from the IgG2 Fc hinge region. Alternatively, a modified Fc for use in the context of the present invention may comprise a variant IgG4 Fc wherein at least one amino acid of the IgG4 Fc hinge region is replaced with the corresponding amino acid from the IgG2 Fc hinge region. Non-limiting, exemplary modified Fc regions that can be used in the context of the present invention are set forth in US Patent Application Publication No. 2014/0243504, the disclosure of which is hereby incorporated by reference in its entirety, as well as any functionally equivalent variants of the modified Fc regions set forth therein.

Other modified Fc domains and Fc modifications that can be used in the context of the present invention include any of the modifications as set forth in US 2014/0171623; U.S. Pat. No. 8,697,396; US 2014/0134162; WO 2014/043361, the disclosures of which are hereby incorporated by reference in their entireties.

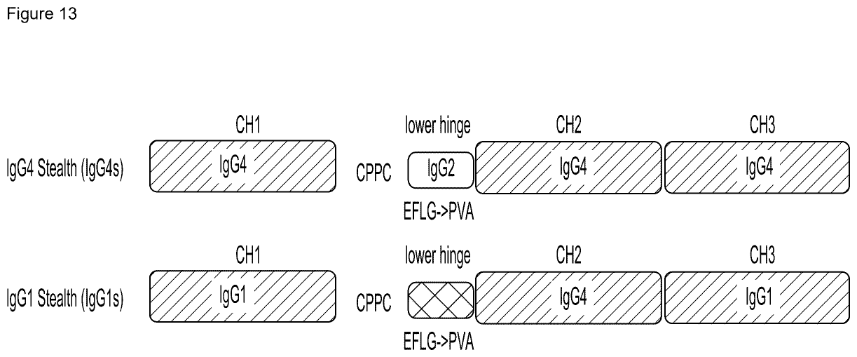

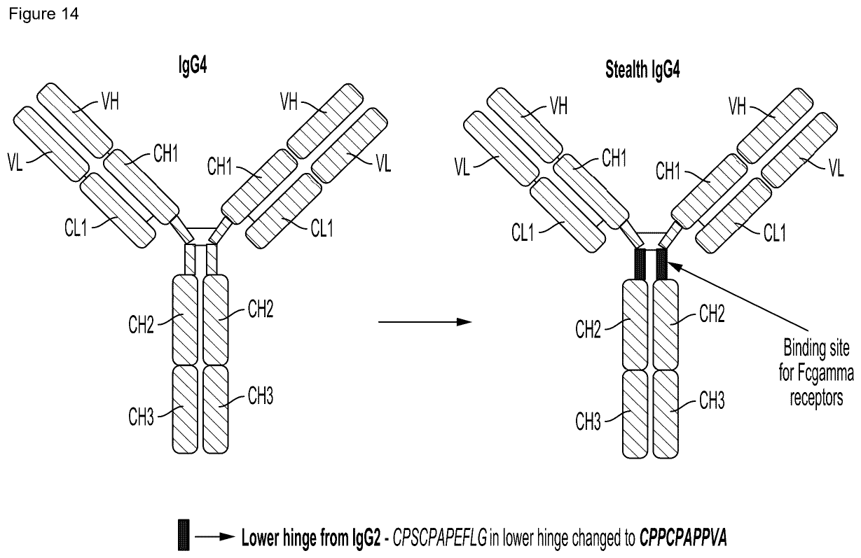

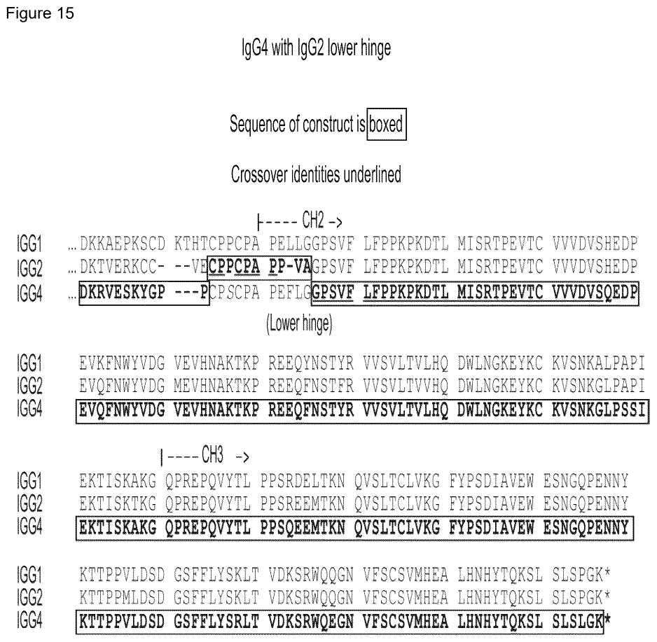

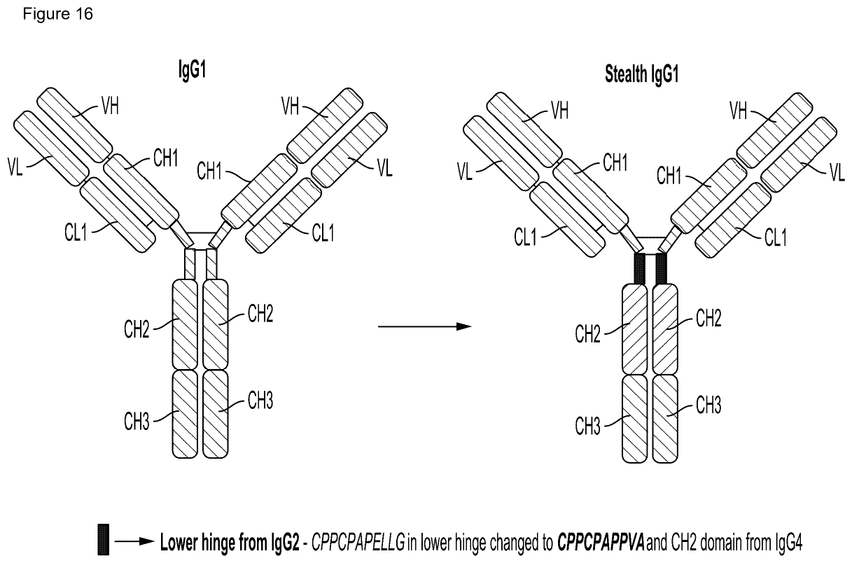

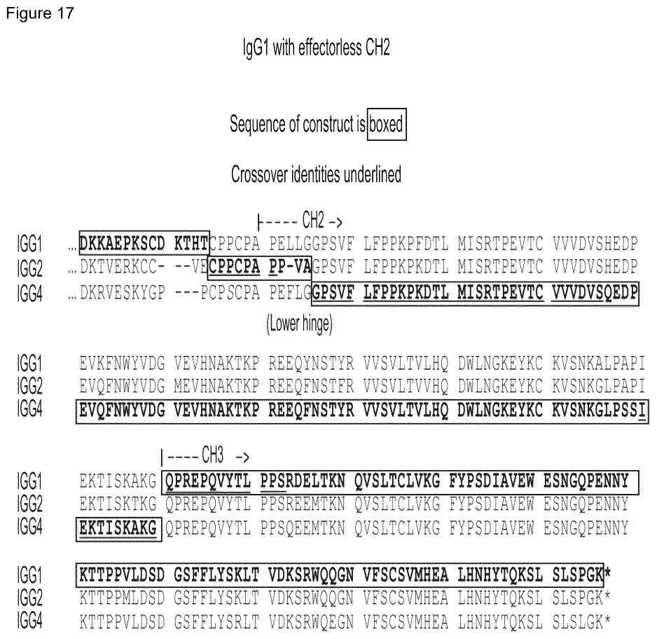

Engineered IgG1 with a stealth mutation is based on the IgG1 framework but with replacement of both the hinge CPPCPAPELLG (amino acids 6-16 of SEQ ID NO: 1) to CPPCPAPPVA (amino acids 6-15 of SEQ ID NO: 2) and the IgG1 CH2 domain with that of IgG4. See e.g., FIG. 13, FIG. 14, FIG. 15, FIG. 16 and FIG. 17.

The engineered IgG4 with a stealth mutation has a three amino acid substitution (EFLG (SEQ ID NO: 95) to PVA) to resemble that of the hinge region of IgG2—which lacks the capacity to bind to FcγR1—in the framework of a hinge stabilized (CPPC (SEQ ID NO: 100) or CPSC (SEQ ID NO: 101)) IgG4, which lacks capacity to bind to C1q. See e.g., FIG. 13, FIG. 14, FIG. 15, FIG. 16 and FIG. 17.

In an embodiment of the invention, a VEGF trap or mini-trap has an IgG1 uber or uber stealth mutation in the hinge region making CPPCPAPELLG (SEQ ID NO: 102) to CPPCPAPGGG (SEQ ID NO: 103); or a VEGF trap or mini-trap has an IgG4 uber or uber stealth mutation in the hinge region making CPPCPAPEFLG (SEQ ID NO: 104) to CPPCPAPGGG (SEQ ID NO: 103).

VEGF trap or mini-trap with human IgG1 stealth Fc or human IgG4 stealth Fc replaces the amino acid residues in the hinge in VEGF Trap IgG1 that are prone to pre-existing anti-hinge antibody responses (PEL). Due to the sequence conservation between the IgG hinges of these chimeric Fc regions to that of human IgG2, they have low responses to pre-existing anti-hinge antibodies (AHA).

Chimeric IgG2/IgG4 Fc VEGF traps and mini-traps are part of the present invention. These molecules combine the favorable properties of both IgG2 and IgG4 isotypes- and possess minimal capacity for effector function. Different IgG isoforms are known to exert different types and levels of effector function. These differences are in large part due to each isoform's ability to interact with the various FcγRs, as well as with complement component C1q. Both the IgG2 and IgG4 isotypes demonstrate diminished capacity for generating Fc dependent effector function when compared to the IgG1 isotype.

The present invention also includes VEGF mini-traps which are the VEGF-binding product of proteolytic cleavage of a VEGF trap of the present invention wherein cleavage removes a portion of the MC. For example, in an embodiment of the invention, the proteolytic cleavage is with IdeS protease (or a variant thereof) or another protease which cleaves prior to GPSV (amino acids 218-221 of SEQ ID NO: 41).

In an embodiment of the invention, a VEGF mini-trap of the present invention comprises an amino acid sequence as set forth below:

(SEQ ID NO: 32)
REGN10103-VEGF mini-trap hlgG2 Fc w/4Cys CPPAPPAPPVA (SEQ ID NO: 105)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLVNRD
LKTQSGSEMKKFLSTLTIDGVTRSDQGLYTCAASSGLMTKKNSTFVRVHEKERKCCVECPPCPAPPVA;
(SEQ ID NO: 33)
REGN10105-VEGF mini-trap hlgG2 Fc w/2Cys CPPAPAPPVA (SEQ ID NO: 106)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLVNRD
LKTQSGSEMKKFLSTLTIDGVTRSDQGLYTCAASSGLMTKKNSTFVRVHEKVECPPCPAPPVA
Mini-REGN10104 (mREGN10104)-VEGF mini-Trap hlgG1 stealth Fc
(SEQ ID NO: 34)
SDTGRPFVEMYSEIPEIIHMTEGRELVIPCRVTSPNITVTLKKFPLDTLIPDGKRIIWDSRKGFIISNATY
KEIGLLTCEATVNGHLYKTNYLTHRQTNTIIDVVLSPSHGIELSVGEKLVLNCTARTELNVGIDENWEYPSSKHQHK
KLVNRDLKTQSGSEMKKELSTLTIDGVTRSDQGLYTCAASSGLMTKKNSTFVRVHEKDKTHTCPPCPAPPVA;

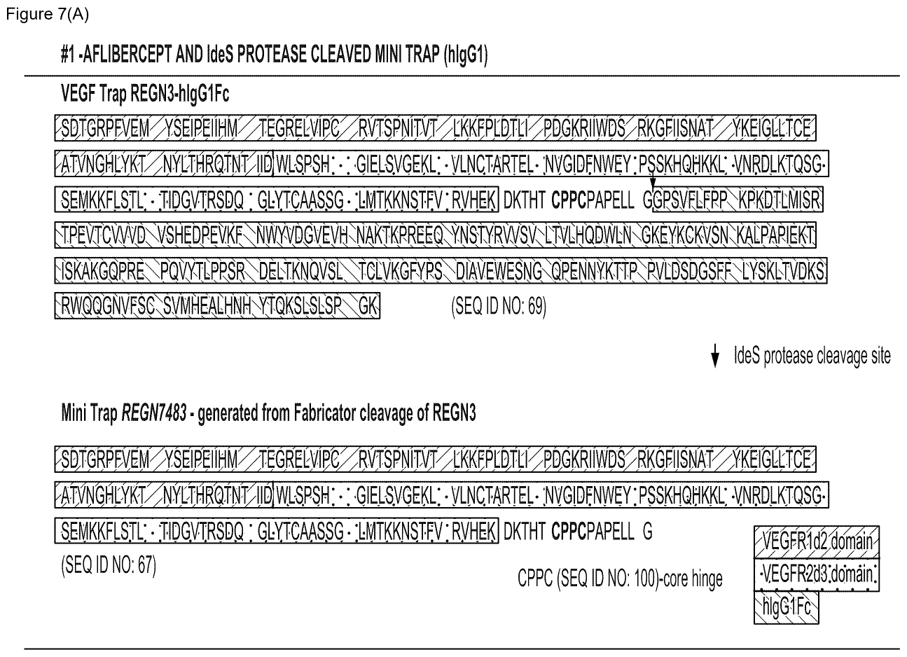

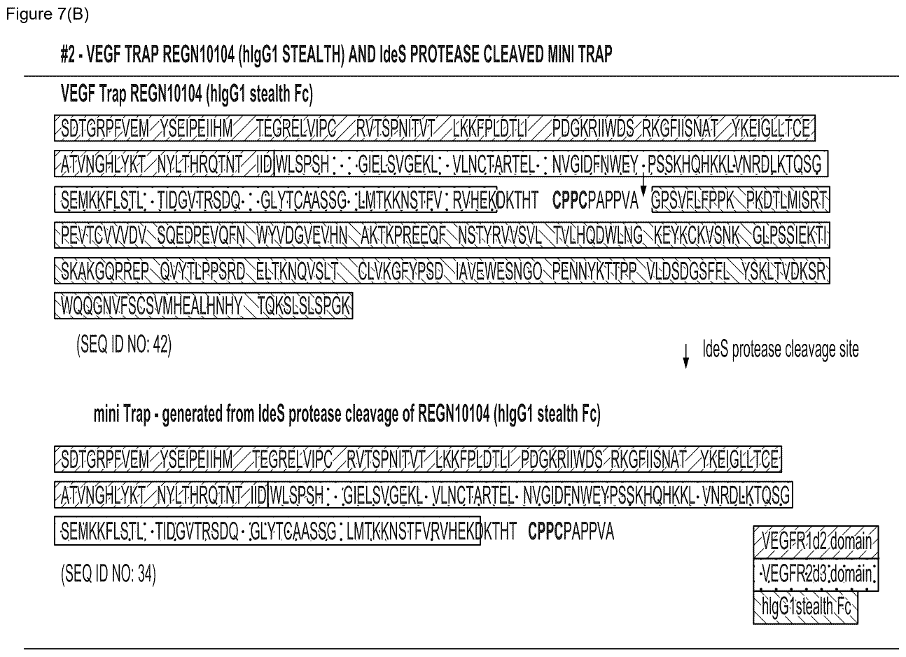

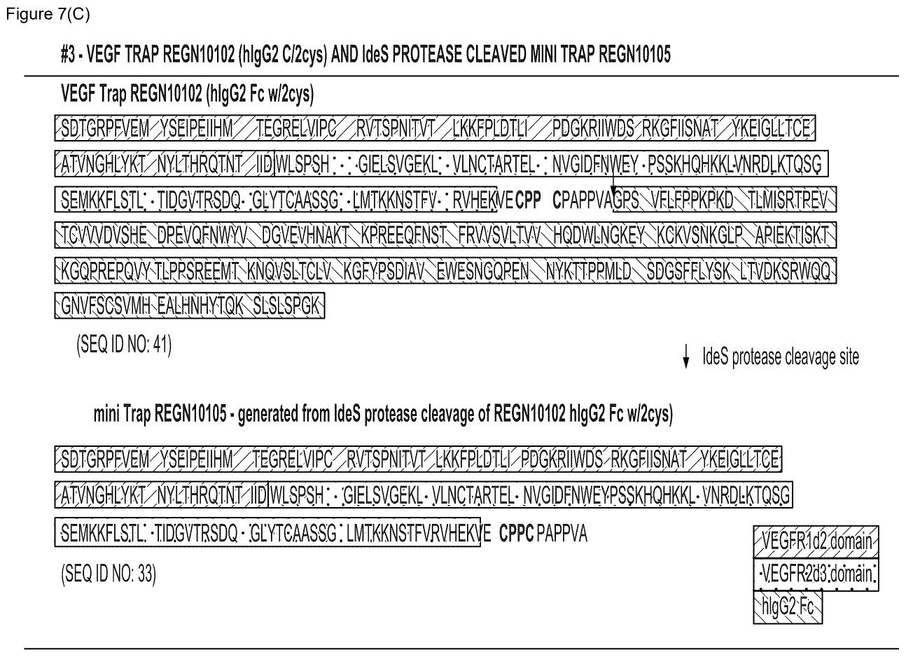

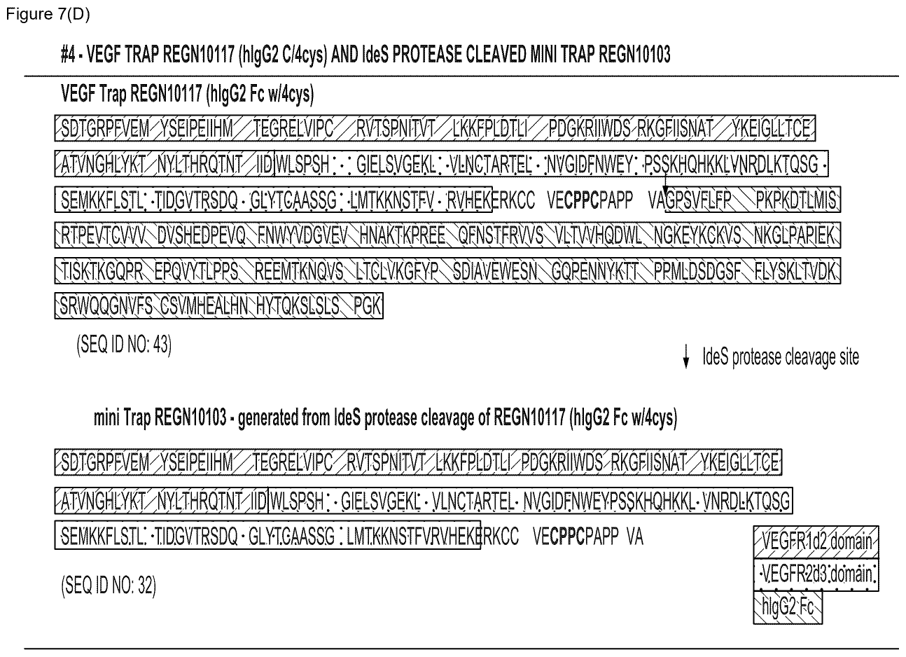

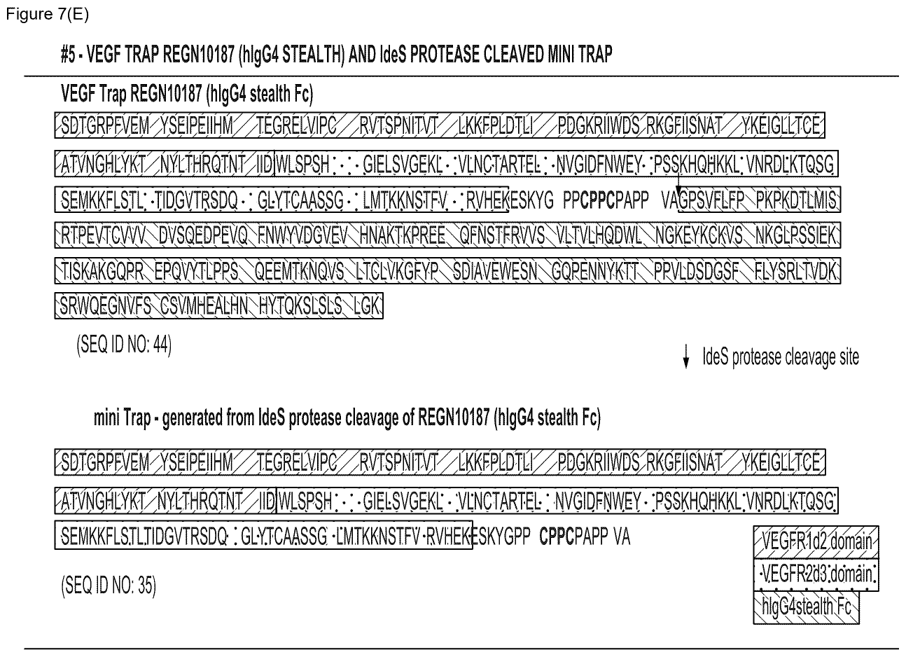

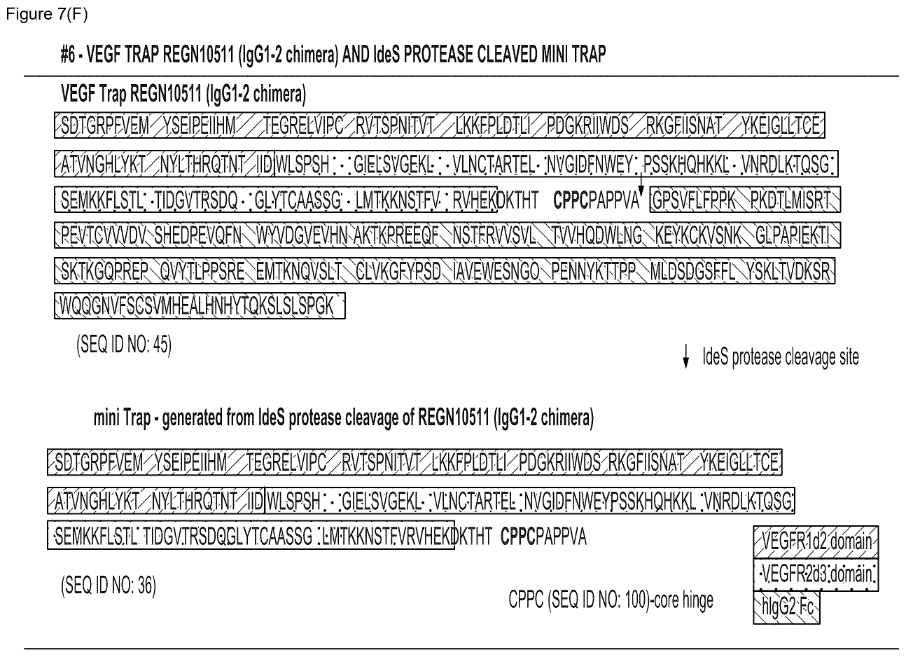

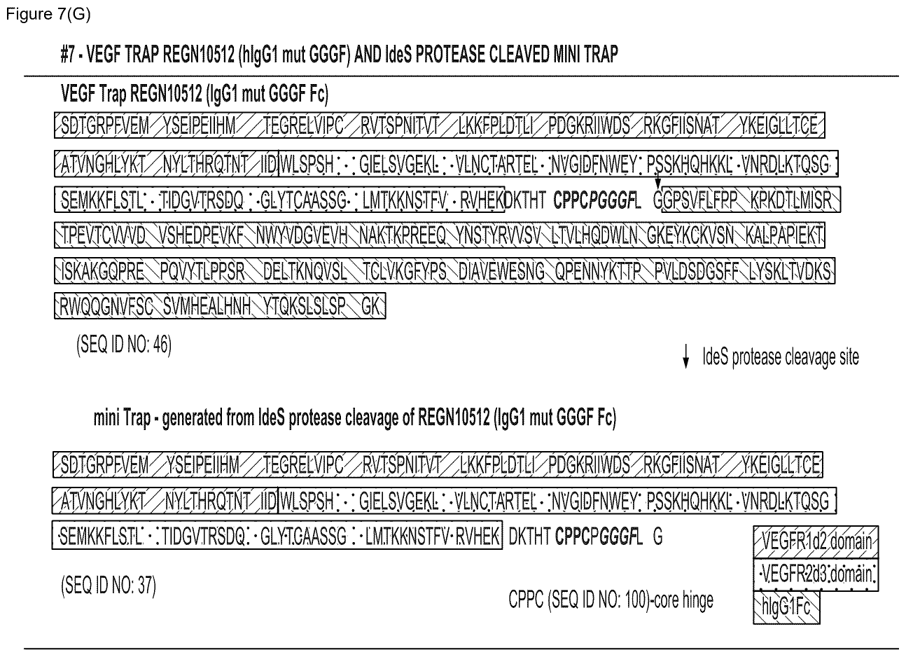

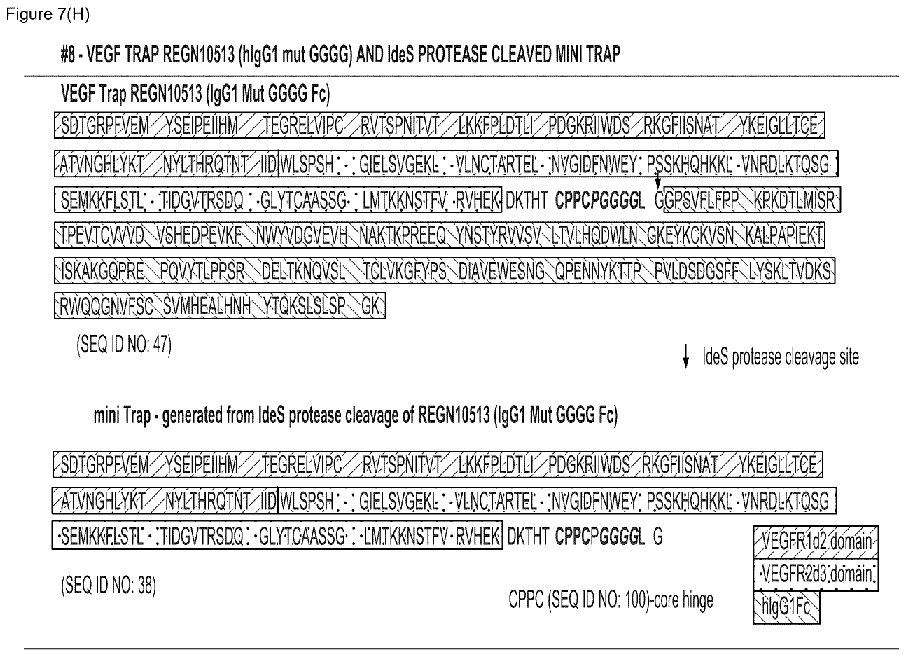

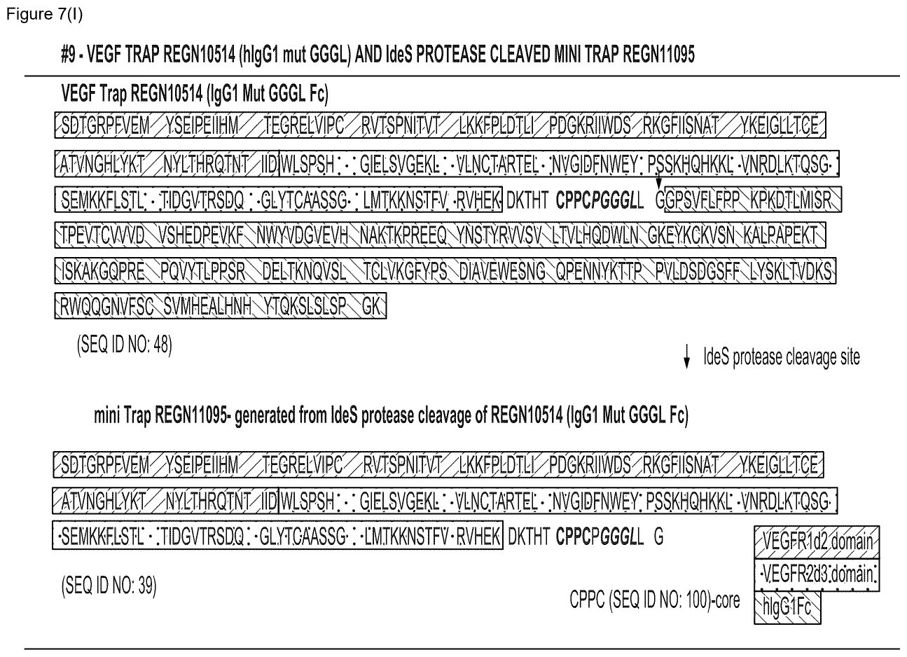

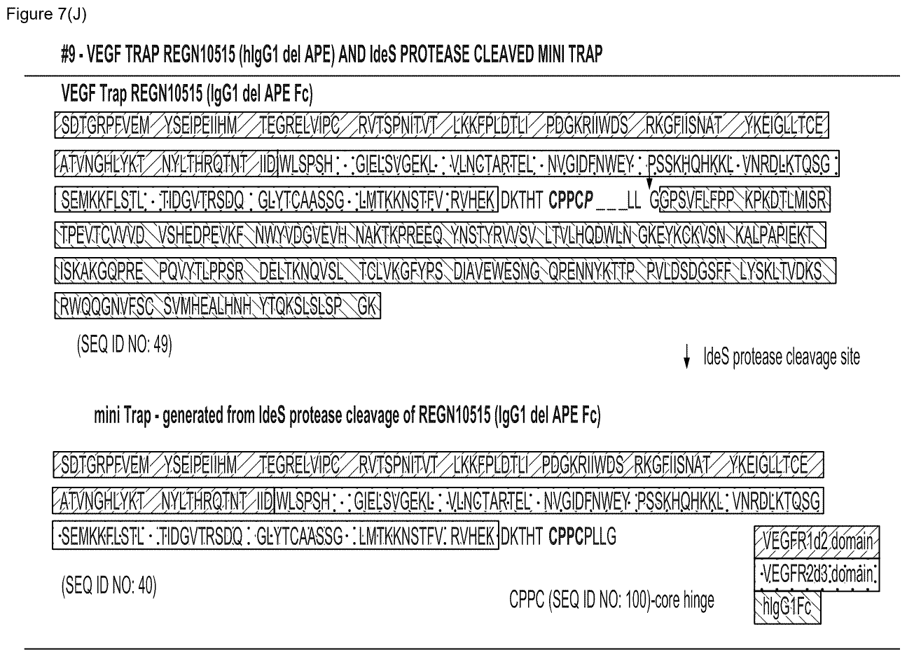

See FIG. 7.

In an embodiment of the invention, a VEGF trap of the present invention comprises an amino acid sequence as set forth below:

REGN10102-VEGF trap hlgG2 Fc w/2Cys
(SEQ ID NO: 41)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLVNRD
LKTQSGSEMKKELSTLTIDGVTRSDQGLYTCAASSGLMTKKNSTFVRVHEKVECPPCPAPPVAGPSVFLFPPKPKDT
LMISRTPEVTCVVVDVSHEDPEVQFNWYVDGVEVHNAKTKPREEQFNSTFRVVSVLTVVHQDWLNGKEYKCKVSNKG
LPAPIEKTISKTKGQPREPQVYTLPPSREEMTKNQVSLTCLVKGFYPSDIAVEWESNGQPENNYKTTPPMLDSDGSF
FLYSKLTVDKSRWQQGNVFSCSVMHEALHNHYTQKSLSLSPGK
REGN10104-VEGF Trap hlgG1 stealth Fc
(SEQ ID NO: 42)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLVNRD
LKTQSGSEMKKELSTLTIDGVTRSDQGLYTCAASSGLMTKKNSTFVRVHEKDKTHTCPPCPAPPVAGPSVFLFPPKP
KDTLMISRTPEVTCVVVDVSQEDPEVQFNWYVDGVEVHNAKTKPREEQFNSTYRVVSVLTVLHQDWLNGKEYKCKVS
NKGLPSSIEKTISKAKGQPREPQVYTLPPSRDELTKNQVSLTCLVKGFYPSDIAVEWESNGQPENNYKTTPPVLDSD
GSFFLYSKLTVDKSRWQQGNVFSCSVMHEALHNHYTQKSLSLSPGK
REGN10117-VEGF Trap hlgG2 Fc w/4 Cys
(SEQ ID NO: 43)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLVNRD
LKTQSGSEMKKELSTLTIDGVTRSDQGLYTCAASSGLMTKKNSTFVRVHEKERKCCVECPPCPAPPVAGPSVFLFPP
KPKDTLMISRTPEVTCVVVDVSHEDPEVQFNWYVDGVEVHNAKTKPREEQFNSTFRVVSVLTVVHQDWLNGKEYKCK
VSNKGLPAPIEKTISKTKGQPREPQVYTLPPSREEMTKNQVSLTCLVKGFYPSDIAVEWESNGQPENNYKTTPPMLD
SDGSFFLYSKLTVDKSRWQQGNVFSCSVMHEALHNHYTQKSLSLSPGK
REGN10187-VEGF Trap hlgG4 stealth Fc
(SEQ ID NO: 44)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLVNRD
LKTQSGSEMKKELSTLTIDGVTRSDQGLYTCAASSGLMTKKNSTFVRVHEKESKYGPPCPPCPAPPVAGPSVFLFPP
KPKDTLMISRTPEVTCVVVDVSQEDPEVQFNWYVDGVEVHNAKTKPREEQFNSTYRVVSVLTVLHQDWLNGKEYKCK
VSNKGLPSSIEKTISKAKGQPREPQVYTLPPSQEEMTKNQVSLTCLVKGFYPSDIAVEWESNGQPENNYKTTPPVLD
SDGSFFLYSRLTVDKSRWQEGNVFSCSVMHEALHNHYTQKSLSLSLGK
REGN10511-VEGF Trap hlgG1-2 Chimera Fc
(SEQ ID NO: 45)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLVNRD
LKTQSGSEMKKFLSTLTIDGVTRSDQGLYTCAASSGLMTKKNSTFVRVHEKDKTHTCPPCPAPPVAGPSVFLFPPKP
KDTLMISRTPEVTCVVVDVSHEDPEVQFNWYVDGVEVHNAKTKPREEQFNSTFRVVSVLTVVHQDWLNGKEYKCKVS
NKGLPAPIEKTISKTKGQPREPQVYTLPPSREEMTKNQVSLTCLVKGFYPSDIAVEWESNGQPENNYKTTPPMLDSD
GSFFLYSKLTVDKSRWQQGNVFSCSVMHEALHNHYTQKSLSLSPGK
REGN10512-VEGF Trap hlgG1 Mut GGGF (SEQ ID NO: 92) Fc
(SEQ ID NO: 46)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLVNRD
LKTQSGSEMKKFLSTLTIDGVTRSDQGLYTCAASSGLMTKKNSTFVRVHEKDKTHTCPPCPGGGFLGGPSVFLFPPK
PKDTLMISRTPEVTCVVVDVSHEDPEVKENWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
REGN10513-VEGF Trap hlgG1 Mut GGGG (SEQ ID NO: 93) Fc
(SEQ ID NO: 47)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLVNRD
LKTQSGSEMKKELSTLTIDGVTRSDQGLYTCAASSGLMTKKNSTFVRVHEKDKTHTCPPCPGGGGLGGPSVFLFPPK
PKDTLMISRTPEVTCVVVDVSHEDPEVKFNWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
REGN10514-VEGF Trap hlgG1 Mut GGGL (SEQ ID NO: 94) Fc
(SEQ ID NO: 48)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLVNRD
LKTQSGSEMKKELSTLTIDGVTRSDQGLYTCAASSGLMTKKNSTFVRVHEKDKTHTCPPCPGGGLLGGPSVFLFPPK
PKDTLMISRTPEVTCVVVDVSHEDPEVKENWYVDGVEVHNAKTKPREEQYNSTYRVVSVLTVLHQDWLNGKEYKCKV
SNKALPAPIEKTISKAKGQPREPQVYTLPPSRDELTKNQVSLTCLVKGFYPSDIAVEWESNGQPENNYKTTPPVLDS
DGSFFLYSKLTVDKSRWQQGNVFSCSVMHEALHNHYTQKSLSLSPGK
REGN10515-VEGF Trap hlgG1 Del (APE) Fc
(SEQ ID NO: 49)
SDTGRPFVEMYSEIPEIIHMTEGRELVIPCRVTSPNITVTLKKFPLDTLIPDGKRIIWDSRKGFIISNATYKEIGLL
TCEATVNGHLYKTNYLTHRQTNTIIDVVLSPSHGIELSVGEKLVLNCTARTELNVGIDENWEYPSSKHQHKKLVNRD
LKTQSGSEMKKFLSTLTIDGVTRSDQGLYTCAASSGLMTKKNSTFVRVHEKDKTHTCPPCPLLGGPSVFLFPPKPKD
TLMISRTPEVTCVVVDVSHEDPEVKENWYVDGVEVHNAKTKPREEQYNSTYRVVSVLTVLHQDWLNGKEYKCKVSNK
ALPAPIEKTISKAKGQPREPQVYTLPPSRDELTKNQVSLTCLVKGFYPSDIAVEWESNGQPENNYKTTPPVLDSDGS
FFLYSKLTVDKSRWQQGNVFSCSVMHEALHNHYTQKSLSLSPGK

Such VEGF Traps are cleavable by IdeS protease. VEGF mini-traps that are the product of such a cleavage are part of the present invention. See FIG. 7.

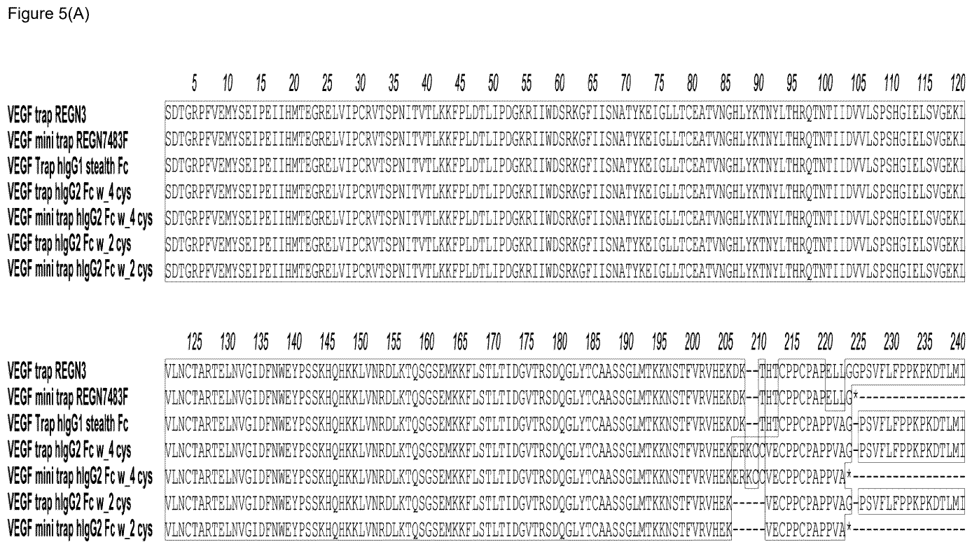

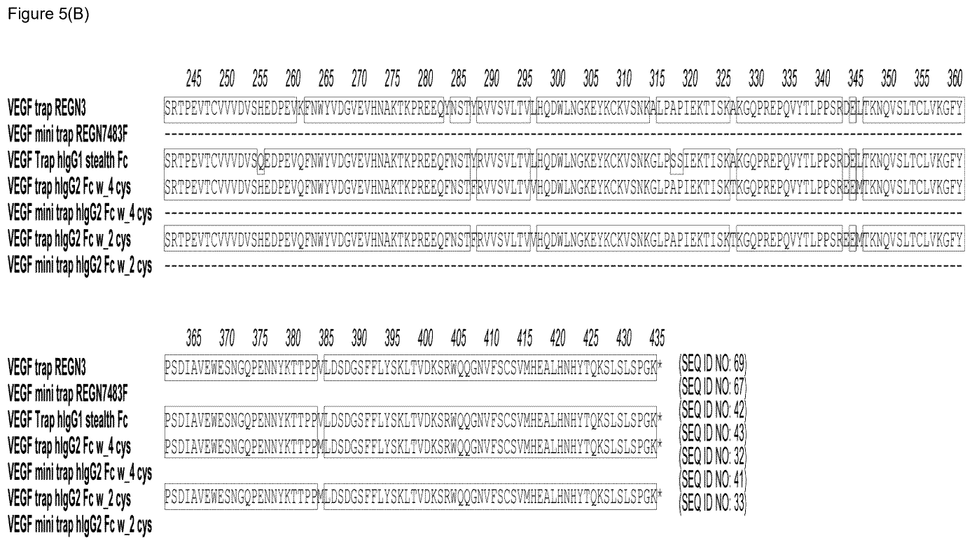

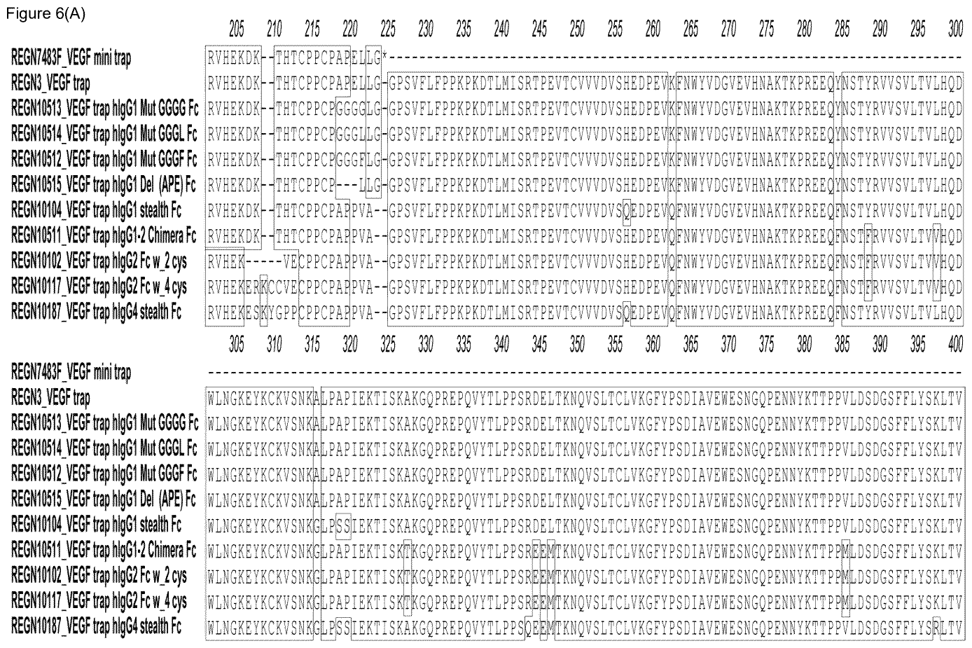

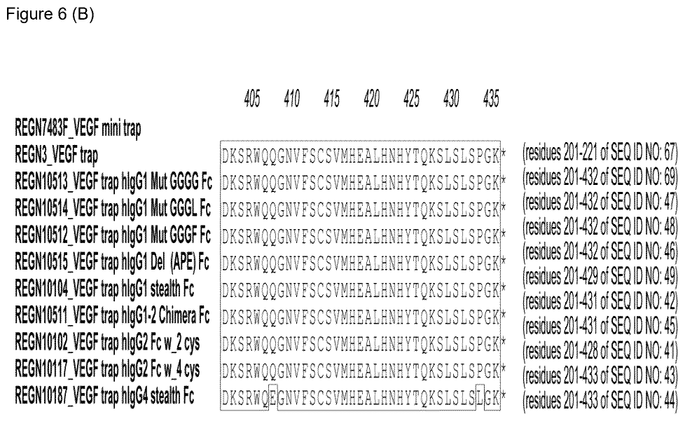

Sequence comparisons between various VEGF traps and VEGF mini-traps are set forth in FIGS. 5 and 6.

A “variant” of a polypeptide (e.g., a variant of a polypeptide including the amino acid sequence of any of SEQ ID Nos: 32-49) refers to a polypeptide comprising an amino acid sequence that is at least about 70-99.9% (e.g., 70, 72, 74, 75, 76, 79, 80, 81, 82, 83, 84, 85, 86, 87, 88, 89, 90, 91, 92, 93, 94, 95, 96, 97, 98, 99, 99.5, 99.9%) identical or similar to a reference amino acid sequence that is set forth herein; when the comparison is performed by a BLAST algorithm wherein the parameters of the algorithm are selected to give the largest match between the respective sequences over the entire length of the respective reference sequences (e.g., expect threshold: 10; word size: 3; max matches in a query range: 0; BLOSUM 62 matrix; gap costs: existence 11, extension 1; conditional compositional score matrix adjustment). In an embodiment of the invention, a variant of a polypeptide refers to a polypeptide comprising about 1, 2, 3, 4, 5, 6, 7, 8, 9 or 10 point mutations (e.g., amino acid substitutions, deletions and/or insertions) relative to that of a reference amino acid sequence that is set forth herein. VEGF traps and mini-traps having amino acid sequences that are variants of any of those set forth herein are part of the present invention.

The following references relate to BLAST algorithms often used for sequence analysis: BLAST ALGORITHMS: Altschul et al. (2005) FEBS J. 272(20): 5101-5109; Altschul, S. F., et al., (1990) J. Mol. Biol. 215:403-410; Gish, W., et al., (1993) Nature Genet. 3:266-272; Madden, T. L., et al., (1996) Meth. Enzymol. 266:131-141; Altschul, S. F., et al., (1997) Nucleic Acids Res. 25:3389-3402; Zhang, J., et al., (1997) Genome Res. 7:649-656; Wootton, J. C., et al., (1993) Comput. Chem. 17:149-163; Hancock, J. M. et al., (1994) Comput. Appl. Biosci. 10:67-70; ALIGNMENT SCORING SYSTEMS: Dayhoff, M. O., et al., “A model of evolutionary change in proteins.” in Atlas of Protein Sequence and Structure, (1978) vol. 5, suppl. 3. M. O. Dayhoff (ed.), pp. 345-352, Natl. Biomed. Res. Found., Washington, D.C.; Schwartz, R. M., et al., “Matrices for detecting distant relationships.” in Atlas of Protein Sequence and Structure, (1978) vol. 5, suppl. 3.” M. O. Dayhoff (ed.), pp. 353-358, Natl. Biomed. Res. Found., Washington, D.C.; Altschul, S. F., (1991) J. Mol. Biol. 219:555-565; States, D. J., et al., (1991) Methods 3:66-70; Henikoff, S., et al., (1992) Proc. Natl. Acad. Sci. USA 89:10915-10919; Altschul, S. F., et al., (1993) J. Mol. Evol. 36:290-300; ALIGNMENT STATISTICS: Karlin, S., et al., (1990) Proc. Natl. Acad. Sci. USA 87:2264-2268; Karlin, S., et al., (1993) Proc. Natl. Acad. Sci. USA 90:5873-5877; Dembo, A., et al., (1994) Ann. Prob. 22:2022-2039; and Altschul, S. F. “Evaluating the statistical significance of multiple distinct local alignments.” in Theoretical and Computational Methods in Genome Research (S. Suhai, ed.), (1997) pp. 1-14, Plenum, New York.

As set forth herein, the terms “REGN10104”; “mREGN10104”; “REGN10102”; “REGN10105”; “REGN10117”; “REGN10103”; “REGN10187”; “mREGN10187”; “REGN10511”; “mREGN10511”; “REGN10512”; “mREGN10512”; “REGN10513”; “mREGN10513”; “REGN10514”; “REGN11095”; “REGN10515”; “mREGN10515” refer to the respective VEGF traps and VEGF mini-traps including polypeptides that include the amino acid sequences described herein under these names as well as variants thereof; and/or multimers (e.g., homodimers) including two or more of such polypeptides.

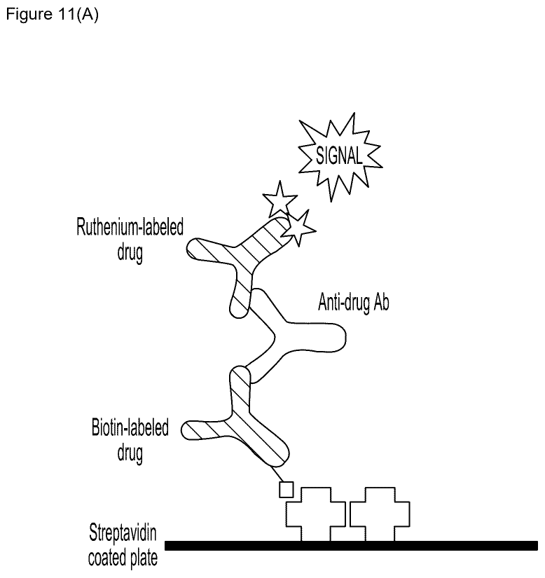

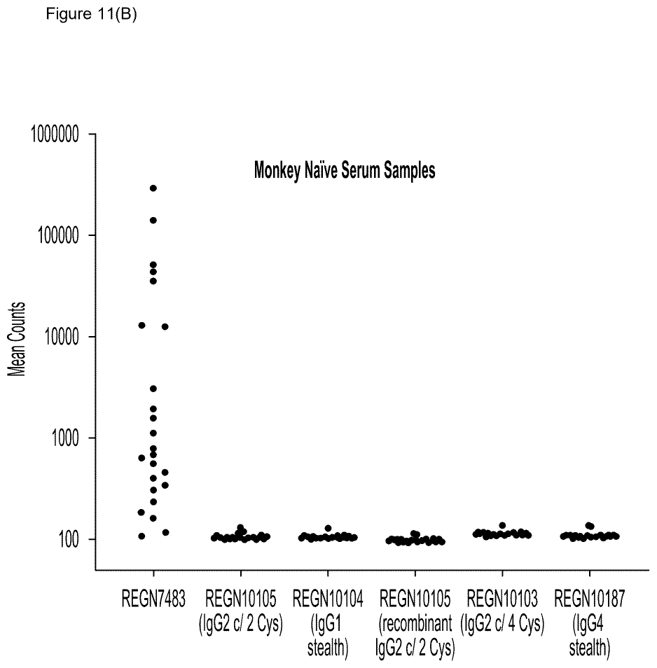

In an embodiment of the invention, a VEGF mini-trap as set forth herein is characterized according to any one or more of the following:
Binds to human VEGF₁₆₅ or VEGF₁₂₁, e.g., to VEGF₁₆₅ with a K_{D} of about 1 or 2 or 1-2 pM (e.g., about 0.74 pM, 0.8, 0.86, 0.865 pM, 1 pM, 1.1 pM, 1.08 pM, 1.78 pM, 2.6 pM, 1.89 pM, 1.37 pM, 1.08 pM, 1.82 pM or 1.37 pM) or a greater affinity, e.g., at 25° C., e.g., wherein said affinity was measured by surface plasmon resonance, e.g., in the presence of about 2.5 nM to about 0.078 pM human VEGF₁₆₅.
Has an IC₅₀ of about 1-2×10⁻¹¹ M for blocking VEGF₁₂₁ and/or VEGF₁₆₅ activation of VEGFR1 and/or VEGFR2, e.g., as measured in HEK293 cells expressing the VEGF receptor (e.g., wherein the extracellular domain is fused to the cytoplasmic domain of IL18Ralpha or IL18Rbeta), e.g., also having an NFkappaB-luciferase-IRES-eGFP reporter gene. For example, an IC₅₀ of about 1-2.5×10⁻¹¹ M for blocking (i) VEGF₁₆₅ (at 40 pM) activation of VEGFR1 (e.g., about 1.06×10⁻¹¹M or about 2.19× 10-11M); (ii) VEGF₁₆₅ (at 20 pM) activation of VEGFR2 (e.g., about 2.46×10⁻¹¹M or about 2.39×10⁻¹¹M); (iii) VEGF₁₂₁ (at 40 pM) activation of VEGFR1 (e.g., about 1.74×10⁻¹¹M or about 2.16×10⁻¹¹M); (iv) VEGF₁₂₁ (at 60 pM) activation of VEGFR2 (e.g., about 1.80×10⁻¹¹M or about 2.30×10⁻¹¹M).
Exhibits less immunogenicity in monkey naïve serum samples and/or human AMD/DME baseline serum samples than that of REGN7483, e.g., as measured by anti-drug antibody (ADA) assay, e.g., electrochemilluminescent immunoassay bridging assay, e.g., wherein said VEGF mini-traps are labeled with biotin and ruthenium, e.g., wherein ADA binds Biotin- and Ruthenium-labeled VEGF mini-trap, forming a bridge, wherein the biotin labeled VEGF mini-trap is bound to a streptavidin-coated plate surface, e.g., wherein an electric current activates Ruthenium-labeled drug to produce an electrochemiluminescent signal that indicates the presence of the complex. See assay format summary in FIG. 11 (A).
Forms a 1:1 complex with VEGF₁₆₅ homodimer (REGN10105: VEGF₁₆₅ homodimer or REGN11095: VEGF₁₆₅ homodimer) (e.g., at 6 μM: 2 μM; 2 μM: 2 μM or 2 μM: 6 μM ratio of the VEGF mini-trap:VEGF₁₆₅ homodimer).
Forms a 1:1:2 complex with VEGF₁₆₅ homodimer and REGN18 anti-VEGF Fab (REGN10105: VEGF₁₆₅ homodimer: REGN18).
Reduces retinal neovascularization when dosed (e.g., at about 0.125, 0.025, 0.25 or 2.5 micrograms per eye) intravitreally in an OIR mouse model (e.g., relative to that of mice dosed with human Fc), e.g., as measured under a retinal flat mount.
Exhibits faster intravitreal half-live (e.g., of about 3.4 or 3.9 days) than aflibercept in rabbit (e.g., New Zealand White rabbits) eyes when intravitreally injected (e.g., at about 0.115, 0.146 or 0.147 micrograms per eye), e.g., wherein the VEGF mini-trap is formulated in 10 mM histidine, e.g., wherein the VEGF mini-trap is conjugated with AF488 dye.
Exhibits less systemic exposure (e.g., plasma levels of free molecule) when injected intravitreally, e.g., at 5.5 mg/eye, than aflibercept (e.g., at 4 mg/eye) (e.g., in non-human primates (NHPs)). For example, having about 0.1 or 0.2 or 0.1-0.2 micrograms/ml (or less) concentration of free molecule in the plasma, e.g., of a NHP, about 336 hours after injection (e.g., a single injection) of about 5.5 mg/eye.
Does not accumulate in the plasma (e.g., as free or VEGF bound molecule), e.g., of a non-human primate, after multiple intravitreal injections (e.g., 4, e.g., about every 28 days), e.g., of about 5.5 mg/eye.
Exhibits greater stability than aflibercept under stress conditions such as elevated temperature, e.g., 37° C., e.g., for 4 weeks under approximately equimolar conditions. For example, at 90 mg/ml develops about 3-4% (e.g., 3.5, 3.6, 3.7, 3.74, 4.0 or 4.1) high molecular weight species (HMW) per week (e.g., for 4 weeks at 37° C.); and/or at 120 mg/ml develops about 5-6% (e.g., 5.0, 5.1, 5.2, 5.3, 5.5, 5.7 or 5.9) high molecular weight species (HMW) per week (e.g., for 4 weeks at 37° C.); for example, whereas aflibercept at 140 mg/ml develops about 5.6% high molecular weight species (HMW) per week (e.g., for 4 weeks at 37° C.); or, for example, whereas REGN7483F develops about 5-6% (e.g., 5.4, 5.45, 5.5, 5.6) HMW species/week (e.g., for 4 weeks at 37° C.) at 90 mg/ml. REGN7483F mini-trap is generated by FabRICATOR cleavage of aflibercept.
Exhibits no significant change in aggregation levels after about 15 minutes of agitation at about 1000 rpm and/or two free-thaw cycles (e.g., at about 90 mg/ml or 120 mg/ml).
Exhibits less viscosity than REGN7483, e.g., about 5.8 at 90 mg/ml or 12.1 cP at 120 mg/ml (e.g., at 20° C.); for example, whereas REGN7483 has a viscosity of about 6.9 cP at 90 mg/ml; or 12.1 cP at 120 mg/ml, or, for example, whereas REGN7483 has a viscosity of about 14.5 cP at 120 mg/ml (e.g., in the presence of histidine at 20° C.)—e.g., wherein the VEGF mini-trap is REGN10105.
Exhibits greater photostability than REGN7483, e.g., developing about 8.7% HMW increase (relative to dark control samples)/M lux*hrs (e.g., wherein the VEGF mini-trap is REGN10105); whereas, for example, REGN7483 exhibits about 9.5% HMW increase (relative to dark control samples)/M lux*hrs, e.g., at about 10 mg/ml concentration, e.g., wherein light is cool white light and/or ultraviolet light.
In a composition comprising VEGF traps and/or VEGF mini-trap (e.g., having an IgG2 hinge region), such molecules are glycosylated with N-glycans (e.g., fucosylated, sialylated, galactosylated, mannosylated or modified with bisecting N-glycan) such that about 43-45% of total N-glycans have a core fucose residue, about 64-72% of total N-glycans have at least one galactose residue, about 20-27% of total N-glycans have at least one sialic acid residue, about 19-25% are identified as having high mannose (e.g., Man5) and/or about 1.6-1.9% of total N-glycans are bisecting glycans; e.g., wherein such molecules are glycosylated with N-glycans such that about 43.3% of total N-glycans have a core fucose residue, about 64.4% of total N-glycans have at least one galactose residue, about 20% of total N-glycans have at least one sialic acid residue, about 25.2% are identified as having high mannose (e.g., Man5) and/or about 1.6% of total N-glycans are bisecting glycans (e.g., wherein the VEGF mini-trap is REGN10103); or such molecules are glycosylated with N-glycans such that about 44.8% of total N-glycans have a core fucose residue, about 71.6% of total N-glycans have at least one galactose residue, about 26.5% of total N-glycans have at least one sialic acid residue, about 18.6% are identified as having high mannose (e.g., Man5) and/or about 1.9% of total N-glycans are bisecting glycans (e.g., wherein the VEGF mini-trap is REGN10105).
one or more serines or threonines of the VEGF trap or mini-trap are O-glycosylated;
one or more asparagines of the VEGF trap or mini-trap are deamidated;
one or more Aspartate-Glycine motifs of the VEGF trap or mini-trap are converted to iso-aspartate-glycine and/or Asn-Gly;
one or more methionines of the VEGF trap or mini-trap are oxidized;
one or more tryptophans of the VEGF trap or mini-trap are converted to N-formylkynurenin;
one or more arginines of the VEGF trap or mini-trap are converted to Arg 3-deoxyglucosone;
the C-terminal residue (e.g., glycine or alanine) of the VEGF trap or mini-trap is not present;
there are one or more non-glycosylated glycosites in the VEGF trap or mini-trap;
the VEGF trap or mini-trap is xylosylated;
the VEGF trap or mini-trap is glycated at a lysine;
the VEGF trap or mini-trap comprises a cystine with a free-thiol group;
the VEGF trap or mini-trap comprises an intrachain disulfide bridge;
the VEGF trap or mini-trap comprises disulfide bridges in parallel or crossed orientation; and/or
the VEGF trap or mini-trap comprises a lysine or arginine which is carboxymethylated.

See e.g., Guidance for Industry Q1B Photostability Testing of New Drug Substances and Products, U.S. Department of Health and Human Services, Food and Drug Administration, Center for Drug Evaluation and Research (CDER), Center for Biologics Evaluation and Research (CBER), November 1996.

The VEGF traps and VEGF mini-traps set forth herein (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) may also be glycosylated (e.g., with N-linked and/or O-linked glycans). For example, in an embodiment of the invention, the VEGF trap or mini-trap is sialylated, galactosylated and/or fusosylated at one or more sites. In an embodiment of the invention, the VEGF trap or mini-trap has one or more N-linked mannose-5 glycans (Man5). In an embodiment of the invention, the VEGF trap or VEGF mini-trap includes glycosylation that is typically added to a molecule which is expressed in a Chinese hamster ovary (CHO) cell (CHO cell glycosylation). See e.g., Carillo et al., Glycosylation Analysis of Therapeutic Glycoproteins Produced in CHO Cells, Methods Mol Biol. 2017; 1603:227-241; or Hossler et al., Optimal and consistent protein glycosylation in mammalian cell culture, Glycobiology 19(9): 936-949 (2009). In an embodiment of the invention, the VEGF trap or VEGF mini-trap is a composition including a heterogeneous mixture of glycosylated variants of the Trap or mini-trap. Individual molecules of VEGF trap or VEGF mini-trap may differ from others in the composition with respect to their particular glycosylation pattern. For example, about 47% of the molecules may be sialyalted, about 70% of the molecules may be galactosylated, about 36% of the molecules may be fucosylated and/or about 11% of the molecules may be Man-5 glycosylated. In an embodiment of the invention, a composition comprising VEGF traps and/or VEGF mini-trap, has the glycan profile of about 43.3% fucosylated, about 64.4% galactosylated, about 20% sialylated, about 25.2% with high mannose glycans and about 1.6% bisecting N-glycans (e.g., wherein the VEGF mini-trap is REGN10103); or having the glycan profile of about 44.8% fucosylated, about 71.6% galactosylated, about 26.5% sialylated, about 18.6% with high mannose glycans and about 1.9% bisecting N-glycans (e.g., wherein the VEGF mini-trap is REGN10105).

See e.g., Yu et al., Production, characterization, and pharmacokinetic properties of antibodies with N-linked mannose-5 glycans, MAbs. July-August 2012; 4(4):475-87.

In an embodiment of the invention, REGN10105 is N-glycosylated (e.g., as described herein) at one or more of the underscored N residues indicated below: SDTGRPFVEMYSEIPEIIHMTEGRELVIPCRVTSPNITVTLKKFPLDTLIPDGKRIIWDSRKGFIISNATYKEIGLL TCEATVNGHLYKTNYLTHRQTNTIIDVVLSPSHGIELSVGEKLVLNCTARTELNVGIDENWEYPSSKHQHKKLVNRD LKTQSGSEMKKFLSTLTIDGVTRSDQGLYTCAASSGLMTKKNSTFVRVHEKVECPPCPAPPVA (SEQ ID NO: 33). Corresponding N residues in the other VEGF traps and mini-traps set forth herein may be similarly N-glycosylated.

In an embodiment of the invention, one or more histidines in a VEGF trap or mini-trap as set forth herein is a 2-oxo-histidine.

Two chemical versions of 2-oxo-histidine (2-oxo-his) can be produced,

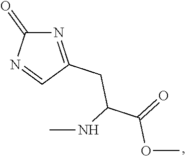

having a 13.98 Da increase in molecular weight relative to histidine (13.98 Da version); or

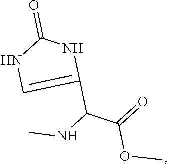

having a 15.99 Da increase in molecular weight relative to histidine (15.99 Da version); wherein the 13.98 Da version of 2-oxo-histidine is the predominant moiety observed in mini-trap expressed in CDM. The content of the 13.98 Da version of 2-oxo-histidine in a peptide can be evaluated spectrophotometrically since this moiety has an enhanced absorbance of light at 350 nM wavelength whereas the 15.99 Da version does not have such an enhanced absorbance. Formation of the 13.98 Da version of 2-oxo-histidine in mini-trap may be catalyzed by light whereas formation of the 15.99 Da version may be catalyzed by metal such as copper (Cu²⁺).

Oxidation of tryptophan can give a complex mixture of products. The primary products can be N-formylkynurenine and kynurenine along with mono-oxidation, di-oxidation and/or tri-oxidation products. Peptides bearing oxidized Trp modifications generally exhibit mass increases of 4, 16, 32 and 48 Da, corresponding to the formation of kynurenine (KYN), hydroxytryptophan (Wₒₓ₁), and N-formylkynurenine/dihydroxytryptophan (NFK/Wₒₓ₂, referred to also as “doubly oxidized Trp”), trihydroxytryptophan (Wₒₓ₃, referred to also as “triply oxidized Trp”), and their combinations, such as hydroxykynurenine (KYNₒₓ₁, +20 Da). Oxidation to hydroxytryptophan (Wₒₓ₁) (Mass spectrometric identification of oxidative modifications of tryptophan residues in proteins: chemical artifact or post-translational modification? J Am Soc Mass Spectrom. 2010 July; 21(7): 1114-1117). Similar to tryptophan, oxidation of tyrosine primarily yields 3,4-dihydroxyphenylalanine (DOPA) and dityrosine (Li, S, C Schoneich, and R T. Borchardt. 1995. Chemical Instability of Protein Pharmaceuticals: Mechanisms of Oxidation and Strategies for Stabilization. Biotechnol. Bioeng. 48:490-500).

The scope of the present invention includes compositions wherein about 0.1-2% (or less, e.g., 0.1% or less, or 0.05% or 0.01%) of histidines in the VEGF mini-traps or traps in the composition are 2-oxo-histidine.

### Ides and Variants Thereof

The present invention includes VEGF mini-traps and compositions thereof that have been produced by proteolytic digestion of aflibercept with Streptococcus pyogenes IdeS (FabRICATOR) and variants thereof. FabRICATOR is commercially available from Genovis, Inc.; Cambridge, MA; Lund, Sweden.

In one embodiment, the IdeS polypeptide comprises an amino acid sequence with at least 70% sequence identity over a full length of the isolated an amino acid sequence as set forth in the group consisting of SEQ ID NO: 50-65. In one aspect, the isolated an amino acid sequence has at least about 80% sequence identity over a full length of the isolated an amino acid sequence. In another aspect, the isolated an amino acid sequence has at least about 90% sequence identity over a full length of the isolated an amino acid sequence. In another aspect, the isolated an amino acid sequence has about 100% sequence identity over a full length of the isolated an amino acid sequence. In one aspect, the polypeptide can be capable of cleaving a target protein into fragments. In a particular aspect, the target protein is an IgG. In another particular aspect, the target protein is a fusion protein. In yet another particular aspect, the fragments can comprise a Fab fragment and/or a Fc fragment.

In one embodiment, the IdeS amino acid sequence comprises a parental amino acid sequence defined by

(SEQ ID NO: 66)
MRKRCYSTSAAVLAAVTLFVLSVDRGVIADSFSANQEIRYSEVTPYHVT
SVWTKGVTPPANFTQGEDVFHAPYVANQGWYDITKTFNGKDDLLCGAAT
AGNMLHWWFDQNKDQIKRYLEEHPEKQKINFNGEQMEDVKEAIDTKNHQ
LDSKLFEYFKEKAFPYLSTKHLGVFPDHVIDMFINGYRLSLTNHGPTPV
KEGSKDPRGGIFDAVFTRGDQSKLLTSRHDFKEKNLKEISDLIKKELTE
GKALGLSHTYANVRINHVINLWGADFDSNGNLKAIYVTDSDSNASIGMK
KYFVGVNSAGKVAISAKEIKEDNIGAQVLGLFTLSTGQDSWNQTN,

but having an asparagine residue at position 87, 130, 182 and/or 274 mutated to an amino acid other than asparagine. In one aspect, the mutation can confer an increased chemical stability at alkaline pH-values compared to the parental amino acid sequence. In another aspect, the mutation can confer an increase in chemical stability by 50% at alkaline pH-values compared to the parental amino acid sequence. In one aspect, the amino acid can be selected from aspartic acid, leucine, and arginine. In a particular aspect, the asparagine residue at position 87 is mutated to aspartic acid residue. In another particular aspect, the asparagine residue at position 130 is mutated to arginine residue. In a yet another particular aspect, the asparagine residue at position 182 is mutated to a leucine residue. In a yet another particular aspect, the asparagine residue at position 274 is mutated to aspartic acid residue. In a yet another particular aspect, the asparagine residue at position 87 and 130 are mutated. In a yet another particular aspect, the asparagine residue at position 87 and 182 are mutated. In a yet another particular aspect, the asparagine residue at position 87 and 274 are mutated. In a yet another particular aspect, the asparagine residue at position 130 and 182 are mutated. In a yet another particular aspect, the asparagine residue at position 130 and 274 are mutated. In a yet another particular aspect, the asparagine residue at position 182 and 274 are mutated. In a yet another particular aspect, the asparagine residue at position 87, 130 and 182 are mutated. In a yet another particular aspect, the asparagine residue at position 87, 182 and 274 are mutated. In a yet another particular aspect, the asparagine residue at position 130, 182 and 274 are mutated. In a yet another particular aspect, the asparagine residue at position 87, 130, 182 and 274 are mutated.

Aflibercept or another VEGF trap can be cleaved by IdeS that has been immobilized to a solid support, e.g., a chromatography bead. For example, a sample including VEGF trap in a buffered aqueous solution (in a cleavage buffer) can be applied to the immobilized IdeS, e.g., in a chromatography column. The column can be incubated, e.g., for 30 minutes, e.g., at about 18° C. The column can then be washed with the cleavage buffer. After cleavage, the digestion and wash solutions can be applied to a protein A column to capture cleaved Fc by-product wherein mini-trap product is retained in the flow-through fraction. In an embodiment of the invention, the cleavage buffer and/or the protein-A column equilibration and wash solutions are at pH 7, e.g., 40 mM Tris, 54 mM Acetate pH 7.0±0.1. Such methods for making a VEGF mini-trap are part of the present invention.

In an embodiment of the invention, an IdeS variant includes an amino acid sequence set forth below:

(SEQ ID NO: 50)
MRKRCYSTSAAVLAAVTLFVLSVDRGVIADSFSANQEIRYSEVTPYHVTSVWTKGVTPPANFTQGEDVFHAPYVANQ
GWYDITKTFNGKDDLLCGAATAGNMLHWWFDQNKDQIKRYLEEHPEKQKINENGEQMFDVKEAIDTKNHQLDSKLFE
YEKEKAFPYLSTKHLGVFPDHVIDMFINGYRLSLTNHGPTPVKEGSKDPRGGIFDAVFTRGDQSKLLTSRHDFKEKN
LKEISDLIKKELTEGKALGLSHTYANVRINHVINLWGADFDSNGNLKAIYVTDSDSNASIGMKKYFVGVNSAGKVAI
SAKEIKEDNIGAQVLGLFTLSTGQDSWNQTN;
(SEQ ID NO: 51)
MRKRCYSTSAAVLAAVTLFVLSVDRGVIADSFSANQEIRYSEVTPYHVTSVWTKGVTPPANFTQGEDVFHA
PYVANQGWYDITKTFDGKDDLLCGAATAGNMLHWWFDQNKDQIKRYLEEHPEKQKINENGEQMFDVKEAIDTKNHQL
DSKLFEYFKEKAFPYLSTKHLGVFPDHVIDMFINGYRLSLTNHGPTPVKEGSKDPRGGIFDAVFTRGDQSKLLTSRH
DFKEKNLKEISDLIKKELTEGKALGLSHTYANVRINHVINLWGADFDSNGNLKAIYVTDSDSNASIGMKKYFVGVNS
AGKVAISAKEIKEDNIGAQVLGLFTLSTGQDSWNQTN;
(SEQ ID NO: 52)
MRKRCYSTSAAVLAAVTLFVLSVDRGVIADSFSANQEIRYSEVTPYHVTSVWTKGVTPPANFTQGEDVFHA
PYVANQGWYDITKTFNGKDDLLCGAATAGNMLHWWFDQNKDQIKRYLEEHPEKQKINFRGEQMFDVKEAIDTKNHQL
DSKLFEYFKEKAFPYLSTKHLGVFPDHVIDMFINGYRLSLTNHGPTPVKEGSKDPRGGIFDAVFTRGDQSKLLTSRH
DFKEKNLKEISDLIKKELTEGKALGLSHTYANVRINHVINLWGADFDSNGNLKAIYVTDSDSNASIGMKKYFVGVNS
AGKVAISAKEIKEDNIGAQVLGLFTLSTGQDSWNQTN;
(SEQ ID NO: 53)
MRKRCYSTSAAVLAAVTLFVLSVDRGVIADSFSANQEIRYSEVTPYHVTSVWTKGVTPPANFTQGEDVFHA
PYVANQGWYDITKTFNGKDDLLCGAATAGNMLHWWFDQNKDQIKRYLEEHPEKQKINENGEQMFDVKEAIDTKNHQL
DSKLFEYFKEKAFPYLSTKHLGVFPDHVIDMFILGYRLSLTNHGPTPVKEGSKDPRGGIFDAVFTRGDQSKLLTSRH
DFKEKNLKEISDLIKKELTEGKALGLSHTYANVRINHVINLWGADFDSNGNLKAIYVTDSDSNASIGMKKYFVGVNS
AGKVAISAKEIKEDNIGAQVLGLFTLSTGQDSWNQTN;
(SEQ ID NO: 54)
MRKRCYSTSAAVLAAVTLFVLSVDRGVIADSFSANQEIRYSEVTPYHVTSVWTKGVTPPANFTQGEDVFHA
PYVANQGWYDITKTFNGKDDLLCGAATAGNMLHWWFDQNKDQIKRYLEEHPEKQKINENGEQMFDVKEAIDTKNHQL
DSKLFEYFKEKAFPYLSTKHLGVFPDHVIDMFINGYRLSLTNHGPTPVKEGSKDPRGGIFDAVFTRGDQSKLLTSRH
DFKEKNLKEISDLIKKELTEGKALGLSHTYANVRINHVINLWGADFDSDGNLKAIYVTDSDSNASIGMKKYFVGVNS
AGKVAISAKEIKEDNIGAQVLGLFTLSTGQDSWNQTN;
(SEQ ID NO: 55)
MRKRCYSTSAAVLAAVTLFVLSVDRGVIADSFSANQEIRYSEVTPYHVTSVWTKGVTPPANFTQGEDVFHA
PYVANQGWYDITKTFDGKDDLLCGAATAGNMLHWWFDQNKDQIKRYLEEHPEKQKINFRGEQMFDVKEAIDTKNHQL
DSKLFEYFKEKAFPYLSTKHLGVFPDHVIDMFINGYRLSLTNHGPTPVKEGSKDPRGGIFDAVFTRGDQSKLLTSRH
DFKEKNLKEISDLIKKELTEGKALGLSHTYANVRINHVINLWGADFDSNGNLKAIYVTDSDSNASIGMKKYFVGVNS
AGKVAISAKEIKEDNIGAQVLGLFTLSTGQDSWNQTN;
(SEQ ID NO: 56)
MRKRCYSTSAAVLAAVTLFVLSVDRGVIADSFSANQEIRYSEVTPYHVTSVWTKGVTPPANFTQGEDVFHA
PYVANQGWYDITKTFDGKDDLLCGAATAGNMLHWWFDQNKDQIKRYLEEHPEKQKINFNGEQMFDVKEAIDTKNHQL
DSKLFEYFKEKAFPYLSTKHLGVFPDHVIDMFILGYRLSLTNHGPTPVKEGSKDPRGGIFDAVFTRGDQSKLLTSRH
DFKEKNLKEISDLIKKELTEGKALGLSHTYANVRINHVINLWGADFDSNGNLKAIYVTDSDSNASIGMKKYFVGVNS
AGKVAISAKEIKEDNIGAQVLGLFTLSTGQDSWNQTN;
(SEQ ID NO: 57)
MRKRCYSTSAAVLAAVTLFVLSVDRGVIADSFSANQEIRYSEVTPYHVTSVWTKGVTPPANFTQGEDVFHA
PYVANQGWYDITKTFDGKDDLLCGAATAGNMLHWWFDQNKDQIKRYLEEHPEKQKINFNGEQMFDVKEAIDTKNHQL
DSKLFEYFKEKAFPYLSTKHLGVFPDHVIDMFINGYRLSLTNHGPTPVKEGSKDPRGGIFDAVFTRGDQSKLLTSRH
DFKEKNLKEISDLIKKELTEGKALGLSHTYANVRINHVINLWGADFDSDGNLKAIYVTDSDSNASIGMKKYFVGVNS
AGKVAISAKEIKEDNIGAQVLGLFTLSTGQDSWNQTN;
(SEQ ID NO: 58)
MRKRCYSTSAAVLAAVTLFVLSVDRGVIADSFSANQEIRYSEVTPYHVTSVWTKGVTPPANFTQGEDVFHA
PYVANQGWYDITKTFNGKDDLLCGAATAGNMLHWWFDQNKDQIKRYLEEHPEKQKINFRGEQMFDVKEAIDTKNHQL
DSKLFEYFKEKAFPYLSTKHLGVFPDHVIDMFILGYRLSLTNHGPTPVKEGSKDPRGGIFDAVFTRGDQSKLLTSRH
DFKEKNLKEISDLIKKELTEGKALGLSHTYANVRINHVINLWGADFDSNGNLKAIYVTDSDSNASIGMKKYFVGVNS
AGKVAISAKEIKEDNIGAQVLGLFTLSTGQDSWNQTN;
(SEQ ID NO: 59)
MRKRCYSTSAAVLAAVTLFVLSVDRGVIADSFSANQEIRYSEVTPYHVTSVWTKGVTPPANFTQGEDVFHA
PYVANQGWYDITKTFNGKDDLLCGAATAGNMLHWWFDQNKDQIKRYLEEHPEKQKINFRGEQMFDVKEAIDTKNHQL
DSKLFEYFKEKAFPYLSTKHLGVFPDHVIDMFINGYRLSLTNHGPTPVKEGSKDPRGGIFDAVFTRGDQSKLLTSRH
DFKEKNLKEISDLIKKELTEGKALGLSHTYANVRINHVINLWGADFDSDGNLKAIYVTDSDSNASIGMKKYFVGVNS
AGKVAISAKEIKEDNIGAQVLGLFTLSTGQDSWNQTN;
(SEQ ID NO: 60)
MRKRCYSTSAAVLAAVTLFVLSVDRGVIADSFSANQEIRYSEVTPYHVTSVWTKGVTPPANFTQGEDVFHA
PYVANQGWYDITKTFNGKDDLLCGAATAGNMLHWWFDQNKDQIKRYLEEHPEKQKINFNGEQMFDVKEAIDTKNHQL
DSKLFEYFKEKAFPYLSTKHLGVFPDHVIDMFILGYRLSLTNHGPTPVKEGSKDPRGGIFDAVFTRGDQSKLLTSRH
DFKEKNLKEISDLIKKELTEGKALGLSHTYANVRINHVINLWGADFDSDGNLKAIYVTDSDSNASIGMKKYFVGVNS
AGKVAISAKEIKEDNIGAQVLGLFTLSTGQDSWNQTN;
(SEQ ID NO: 61)
MRKRCYSTSAAVLAAVTLFVLSVDRGVIADSFSANQEIRYSEVTPYHVTSVWTKGVTPPANFTQGEDVFHA
PYVANQGWYDITKTFDGKDDLLCGAATAGNMLHWWFDQNKDQIKRYLEEHPEKQKINFRGEQMFDVKEAIDTKNHQL
DSKLFEYFKEKAFPYLSTKHLGVFPDHVIDMFILGYRLSLTNHGPTPVKEGSKDPRGGIFDAVFTRGDQSKLLTSRH
DFKEKNLKEISDLIKKELTEGKALGLSHTYANVRINHVINLWGADFDSNGNLKAIYVTDSDSNASIGMKKYFVGVNS
AGKVAISAKEIKEDNIGAQVLGLFTLSTGQDSWNQTN;
(SEQ ID NO: 62)
MRKRCYSTSAAVLAAVTLFVLSVDRGVIADSFSANQEIRYSEVTPYHVTSVWTKGVTPPANFTQGEDVFHA
PYVANQGWYDITKTFDGKDDLLCGAATAGNMLHWWFDQNKDQIKRYLEEHPEKQKINFRGEQMFDVKEAIDTKNHQL
DSKLFEYFKEKAFPYLSTKHLGVFPDHVIDMFINGYRLSLTNHGPTPVKEGSKDPRGGIFDAVFTRGDQSKLLTSRH
DFKEKNLKEISDLIKKELTEGKALGLSHTYANVRINHVINLWGADFDSDGNLKAIYVTDSDSNASIGMKKYFVGVNS
AGKVAISAKEIKEDNIGAQVLGLFTLSTGQDSWNQTN;
(SEQ ID NO: 63)
MRKRCYSTSAAVLAAVTLFVLSVDRGVIADSFSANQEIRYSEVTPYHVTSVWTKGVTPPANFTQGEDVFHA
PYVANQGWYDITKTFDGKDDLLCGAATAGNMLHWWFDQNKDQIKRYLEEHPEKQKINFNGEQMFDVKEAIDTKNHQL
DSKLFEYFKEKAFPYLSTKHLGVFPDHVIDMFILGYRLSLTNHGPTPVKEGSKDPRGGIFDAVFTRGDQSKLLTSRH
DFKEKNLKEISDLIKKELTEGKALGLSHTYANVRINHVINLWGADFDSDGNLKAIYVTDSDSNASIGMKKYFVGVNS
AGKVAISAKEIKEDNIGAQVLGLFTLSTGQDSWNQTN;
(SEQ ID NO: 64)
MRKRCYSTSAAVLAAVTLFVLSVDRGVIADSFSANQEIRYSEVTPYHVTSVWTKGVTPPANFTQGEDVFHAPYVANQ
GWYDITKTFNGKDDLLCGAATAGNMLHWWFDQNKDQIKRYLEEHPEKQKINFRGEQMFDVKEAIDTKNHQLDSKLFE
YFKEKAFPYLSTKHLGVFPDHVIDMFILGYRLSLTNHGPTPVKEGSKDPRGGIFDAVFTRGDQSKLLTSRHDFKEKN
LKEISDLIKKELTEGKALGLSHTYANVRINHVINLWGADFDSDGNLKAIYVTDSDSNASIGMKKYFVGVNSAGKVAI
SAKEIKEDNIGAQVLGLFTLSTGQDSWNQTN;
or
(SEQ ID NO: 65)
MRKRCYSTSAAVLAAVTLFVLSVDRGVIADSFSANQEIRYSEVTPYHVTSVWTKGVTPPANFTQGEDVFHA
PYVANQGWYDITKTFDGKDDLLCGAATAGNMLHWWFDQNKDQIKRYLEEHPEKQKINFRGEQMFDVKEAIDTKNHQL
DSKLFEYFKEKAFPYLSTKHLGVFPDHVIDMFILGYRLSLTNHGPTPVKEGSKDPRGGIFDAVFTRGDQSKLLTSRH
DFKEKNLKEISDLIKKELTEGKALGLSHTYANVRINHVINLWGADFDSDGNLKAIYVTDSDSNASIGMKKYFVGVNS
AGKVAISAKEIKEDNIGAQVLGLFTLSTGQDSWNQTN.

Such IdeS variants possess an increased chemical stability at alkaline pH-values compared to the parental amino acid sequence (SEQ ID NO: 66).

The present invention includes compositions including a VEGF mini-trap as set forth herein and the corresponding VEGF trap which was used to generate the VEGF mini-trap product through IdeS cleavage. The VEGF trap in the mixture is undigested or yet-to-be digested reactant for proteolysis by IdeS. For example, the composition may include:
VEGF Trap REGN10102 and VEGF mini-trap REGN10105;
VEGF Trap REGN10104 and VEGF mini-trap mREGN10104;
VEGF Trap REGN10117 and VEGF mini-trap REGN10103;
VEGF Trap REGN10187 and VEGF mini-trap mREGN10187;
VEGF Trap REGN10511 and VEGF mini-trap mREGN10511;
VEGF Trap REGN10512 and VEGF mini-trap mREGN10512;
VEGF Trap REGN10513 and VEGF mini-trap mREGN10513;
VEGF Trap REGN10514 and VEGF mini-trap REGN11095; and/or.
VEGF Trap REGN10515 and VEGF mini-trap mREGN10515.
   In an embodiment of the invention, there is more (e.g., moles or grams) of the VEGF mini-trap than the VEGF trap, for example, 2, 5, 10 or 15 times more. Such a composition may further include IdeS or a variant thereof, e.g., as set forth herein. IdeS may be either absent or only be present at trace amounts, e.g., 0.04 ppm (or less) or less than 0.3 ppm. The composition may also include a buffer and/or other components conducive to protease cleavage.

### Polynucleotides, Vectors, Host Cells and Methods of Making

An isolated polynucleotide encoding any VEGF trap or VEGF mini-trap polypeptides set forth herein forms part of the present invention as does a vector comprising the polynucleotide and/or a host cell (e.g., Chinese hamster ovary (CHO) cell) comprising the polynucleotide, vector, VEGF trap or VEGF mini-trap and/or a polypeptide set forth herein. Such host cells also form part of the present invention.

A polynucleotide includes DNA and RNA. The present invention includes any polynucleotide of the present invention, for example, encoding a VEGF trap or VEGF mini-trap polypeptide set forth herein (e.g., any of SEQ ID NOs: 32-49). Optionally, the polynucleotide is operably linked to a promoter or other expression control sequence. In an embodiment of the invention, a polynucleotide of the present invention is fused to a secretion signal sequence. Polypeptides encoded by such polynucleotides are also within the scope of the present invention.

In an embodiment of the invention, the polynucleotide encoding REGN10103 comprises the nucleotide sequence:

(SEQ ID NO: 71)
AGTGATACCGGTAGACCTTTCGTAGAGATGTACAGTGAAATCCCCGAAA
TTATACACATGACTGAAGGAAGGGAGCTCGTCATTCCCTGCCGGGTTAC
GTCACCTAACATCACTGTTACTTTAAAAAAGTTTCCACTTGACACTTTG
ATCCCTGATGGAAAACGCATAATCTGGGACAGTAGAAAGGGCTTCATCA
TATCAAATGCAACGTACAAAGAAATAGGGCTTCTGACCTGTGAAGCAAC
AGTCAATGGGCATTTGTATAAGACAAACTATCTCACACATCGACAAACC
AATACAATCATAGATGTGGTTCTGAGTCCGTCTCATGGAATTGAACTAT
CTGTTGGAGAAAAGCTTGTCTTAAATTGTACAGCAAGAACTGAACTAAA
TGTGGGGATTGACTTCAACTGGGAATACCCTTCTTCGAAGCATCAGCAT
AAGAAACTTGTAAACCGAGACCTAAAAACCCAGTCTGGGAGTGAGATGA
AGAAATTTTTGAGCACCTTAACTATAGATGGTGTAACCCGGAGTGACCA
AGGATTGTACACCTGTGCAGCATCCAGTGGGCTGATGACCAAGAAGAAC
AGCACATTTGTCAGGGTCCATGAAAAGGAGCGCAAATGTTGTGTCGAGT
GCCCACCGTGCCCAGCACCACCTGTGGCATGA

In an embodiment of the invention, the polynucleotide encoding REGN10105 comprises the nucleotide sequence:

(SEQ ID NO: 72)
AGTGATACCGGTAGACCTTTCGTAGAGATGTACAGTGAAATCCCCGAAA
TTATACACATGACTGAAGGAAGGGAGCTCGTCATTCCCTGCCGGGTTAC
GTCACCTAACATCACTGTTACTTTAAAAAAGTTTCCACTTGACACTTTG
ATCCCTGATGGAAAACGCATAATCTGGGACAGTAGAAAGGGCTTCATCA
TATCAAATGCAACGTACAAAGAAATAGGGCTTCTGACCTGTGAAGCAAC
AGTCAATGGGCATTTGTATAAGACAAACTATCTCACACATCGACAAACC
AATACAATCATAGATGTGGTTCTGAGTCCGTCTCATGGAATTGAACTAT
CTGTTGGAGAAAAGCTTGTCTTAAATTGTACAGCAAGAACTGAACTAAA
TGTGGGGATTGACTTCAACTGGGAATACCCTTCTTCGAAGCATCAGCAT
AAGAAACTTGTAAACCGAGACCTAAAAACCCAGTCTGGGAGTGAGATGA
AGAAATTTTTGAGCACCTTAACTATAGATGGTGTAACCCGGAGTGACCA
AGGATTGTACACCTGTGCAGCATCCAGTGGGCTGATGACCAAGAAGAAC
AGCACATTTGTCAGGGTCCATGAAAAGGTCGAGTGCCCACCGTGCCCAG
CACCACCTGTGGCATGA

In an embodiment of the invention, the polynucleotide encoding REGN11095 comprises the nucleotide sequence:

(SEQ ID NO: 73)
AGTGATACCGGTAGACCTTTCGTAGAGATGTACAGTGAAATCCCCGAAA
TTATACACATGACTGAAGGAAGGGAGCTCGTCATTCCCTGCCGGGTTAC
GTCACCTAACATCACTGTTACTTTAAAAAAGTTTCCACTTGACACTTTG
ATCCCTGATGGAAAACGCATAATCTGGGACAGTAGAAAGGGCTTCATCA
TATCAAATGCAACGTACAAAGAAATAGGGCTTCTGACCTGTGAAGCAAC
AGTCAATGGGCATTTGTATAAGACAAACTATCTCACACATCGACAAACC
AATACAATCATAGATGTGGTTCTGAGTCCGTCTCATGGAATTGAACTAT
CTGTTGGAGAAAAGCTTGTCTTAAATTGTACAGCAAGAACTGAACTAAA
TGTGGGGATTGACTTCAACTGGGAATACCCTTCTTCGAAGCATCAGCAT
AAGAAACTTGTAAACCGAGACCTAAAAACCCAGTCTGGGAGTGAGATGA
AGAAATTTTTGAGCACCTTAACTATAGATGGTGTAACCCGGAGTGACCA
AGGATTGTACACCTGTGCAGCATCCAGTGGGCTGATGACCAAGAAGAAC
AGCACATTTGTCAGGGTCCATGAAAAGGACAAAACTCACACATGCCCAC
CGTGCCCAGGCGGTGGACTTCTAGGGTGA

In general, a “promoter” or “promoter sequence” is a DNA regulatory region capable of binding an RNA polymerase in a cell (e.g., directly or through other promoter-bound proteins or substances) and initiating transcription of a coding sequence. A promoter may be operably linked to other expression control sequences, including enhancer and repressor sequences and/or with a polynucleotide of the invention. Promoters which may be used to control gene expression include, but are not limited to, cytomegalovirus (CMV) promoter (U.S. Pat. Nos. 5,385,839 and 5,168,062), the SV40 early promoter region (Benoist, et al., (1981) Nature 290:304-310), the promoter contained in the 3′ long terminal repeat of Rous sarcoma virus (Yamamoto, et al., (1980) Cell 22:787-797), the herpes thymidine kinase promoter (Wagner, et al., (1981) Proc. Natl. Acad. Sci. USA 78:1441-1445), the regulatory sequences of the metallothionein gene (Brinster, et al., (1982) Nature 296:39-42); prokaryotic expression vectors such as the beta-lactamase promoter (VIIIa-Komaroff, et al., (1978) Proc. Natl. Acad. Sci. USA 75:3727-3731), or the tac promoter (DeBoer, et al., (1983) Proc. Natl. Acad. Sci. USA 80:21-25); see also “Useful proteins from recombinant bacteria” in Scientific American (1980) 242:74-94; and promoter elements from yeast or other fungi such as the Gal4 promoter, the ADC (alcohol dehydrogenase) promoter, PGK (phosphoglycerol kinase) promoter or the alkaline phosphatase promoter.

A polynucleotide encoding a polypeptide is “operably linked” to a promoter or other expression control sequence when, in a cell or other expression system, the sequence directs RNA polymerase mediated transcription of the coding sequence into RNA, preferably mRNA, which then may be RNA spliced (if it contains introns) and, optionally, translated into a protein encoded by the coding sequence.

The present invention includes polynucleotides encoding VEGF trap or VEGF mini-trap polypeptide chains which are variants of those whose amino acid sequence is specifically set forth herein (e.g., any of SEQ ID NOs: 32-49).

Eukaryotic and prokaryotic host cells, including mammalian cells, may be used as hosts for expression of a VEGF trap or VEGF mini-trap polypeptide (e.g., any of SEQ ID NOs: 32-49). Such host cells are well known in the art and many are available from the American Type Culture Collection (ATCC). These host cells include, inter alia, Chinese hamster ovary (CHO) cells, CHO K1, EESYR, NICE, NSO, Sp2/0, embryonic kidney cells and BHK cells. Host cells include fungal cells such as Pichia, e.g., Pichia pastoris or bacterial cells, e.g., E. coli. The present invention includes an isolated host cell (e.g., a CHO cell or any type of host cell set forth above) comprising one or more VEGF trap or VEGF mini-trap polypeptides (or variant thereof) and/or a polynucleotide encoding such a polypeptide(s) (e.g., as discussed herein). A polynucleotide encoding a VEGF trap or VEGF mini-trap, or vector thereof, may be ectopic or chromosomally integrated into the chromosomal DNA or the host cell.

Transformation can be by any known method for introducing polynucleotides into a host cell. Methods for introduction of heterologous polynucleotides into mammalian cells are well known in the art and include dextran-mediated transfection, calcium phosphate precipitation, polybrene-mediated transfection, protoplast fusion, electroporation, encapsulation of the polynucleotide(s) in liposomes, biolistic injection and direct microinjection of the DNA into nuclei. In addition, nucleic acid molecules may be introduced into mammalian cells by viral vectors. Methods of transforming cells are well known in the art. See, for example, U.S. Pat. Nos. 4,399,216; 4,912,040; 4,740,461 and 4,959,455. Thus, the present invention includes recombinant methods for making a VEGF trap or VEGF mini-trap comprising the steps of:
(i) culturing the host cell (e.g., CHO or Pichia or Pichia pastoris) under conditions favorable to expression of a polynucleotide encoding a VEGF trap or VEGF mini-trap (e.g., in a bioreactor, e.g., in chemically defined medium (CDM) or non-CDM) and,
(ii) optionally, isolating the VEGF trap or VEGF mini-trap or chain thereof from the host cell and/or medium in which the host cell is grown.

In an embodiment of the invention, the method includes the step of introducing, into a host cell, one or more polynucleotides encoding a VEGF trap or VEGF mini-trap polypeptide (e.g., including the amino acid sequence set forth in any one of SEQ ID NOs: 32-49), for example, wherein the polynucleotide is in a vector; and/or integrates into the host cell chromosome and/or is operably linked to a promoter. When making a VEGF trap or VEGF mini-trap that includes two or more polypeptide chains, co-expression of the chains in a single host cell leads to association of the chains, e.g., in the cell or on the cell surface or outside the cell if such chains are secreted, so as to form homodimeric VEGF mini-trap. The present invention also includes VEGF traps or VEGF mini-traps which are the product of the production methods set forth herein, and, optionally, the purification methods set forth herein.

Recombinant VEGF traps and VEGF mini-traps (e.g., any of SEQ ID NOs: 32-49) are part of the present invention, e.g., that are the product of such a method.

See e.g., Ling et al., Development and manufacturability assessment of chemically-defined medium for the production of protein therapeutics in CHO cells, Biotechnol Prog September-October 2015; 31(5):1163-71.

The present invention also provides a method for making a VEGF trap or VEGF mini-trap (e.g., a homodimeric VEGF mini-trap) set forth herein, from a VEGF trap, comprising, consisting of or consisting essentially of proteolyzing VEGF Trap with a protease which cleaves the VEGF Trap in the immunoglobulin Fc multimerizing component below (to the C-terminal side of) the Fc hinge domain such that the lower hinge or a portion thereof is part of the VEGF mini-trap product. For example, the proteolysis can be done with S. pyogenes IdeS (e.g., FabRICATOR protease; Genovis, Inc.; Cambridge, MA; Lund, Sweden) or Streptococcus equi subspecies zooepidemicus IdeZ (New England Biolabs; Ipswich, MA). In an embodiment of the invention, such a method lacks any steps that include significant modification of the amino acid residues of such VEGF mini-trap polypeptide (e.g., directed chemical modification such as PEGylation or iodoacetamidation) and/or disulfide bridge reduction. A VEGF mini-trap product of such a method for making is also part of the present invention.

Such a method for making a VEGF mini-trap may be followed by a method for purifying VEGF mini-trap, e.g., from contaminants such as an Fc fragment, proteolytic enzyme or other material. In an embodiment of the invention, the method for purifying is done under conditions promoting the formation of homodimeric VEGF mini-trap (e.g., under non-reducing conditions, e.g., in the absence of reducing agents such as dithiothreitol (DTT) or beta-mercaptoethanol). The VEGF mini-trap product of such a method for making and/or a method for purifying is also part of the present invention. In an embodiment of the invention, purification is performed by a method including chromatographic purification.

Also provided by the present invention is an isolated polypeptide comprising, consisting or consisting essentially an amino acid sequence selected from the group consisting of SEQ ID Nos: 32-49. Compositions comprising such polypeptides, e.g., in which all or a portion of such polypeptides are associated, e.g., into homodimeric VEGF trap or VEGF mini-trap complexes which can bind to VEGF are part of the present invention. Such compositions may include, for example, one or more of host cells, protease (e.g., IdeS) and culture medium.

Bioreactors, such as tank bioreactors or single-use bioreactors, may be used to culture host cells for expression of a VEGF trap or VEGF mini-trap. Such bioreactors may include an impellor (e.g., turbine, marine or spiral design) for culture mixing and means for controlling temperature, pH, oxygen and/or nitrogen content in the culture medium. Bioreactor volumes may be research scale (e.g., 250 mL or 2 liters) or manufacturing scale (e.g., 2000 liters or 10000 liters). The present invention includes a tank bioreactor that comprises a VEGF trap and/or VEGF mini-trap of the invention (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) and, optionally, host cells (e.g., as discussed herein) and/or culture medium (e.g., CDM). See e.g., Innovations in Cell Culture, BioProcess Vol. 12 (suppl 5) (September 2014).

### Combinations and Pharmaceutical Formulations

The present invention provides compositions that include VEGF traps or VEGF mini-traps (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) in association with one or more ingredients; as well as methods of use thereof and methods of making such compositions. Pharmaceutic formulations comprising a VEGF mini-trap and a pharmaceutically acceptable carrier or excipient are part of the present invention. In an embodiment of the invention, a pharmaceutical formulation of the present invention has a pH of approximately 5.5, 5.6, 5.7, 5.8, 5.9, 6.0, 6.1 or 6.2.

To prepare pharmaceutical formulations of the VEGF traps or VEGF mini-traps (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515), the mini-traps are admixed with a pharmaceutically acceptable carrier or excipient. See, e.g., Remington's Pharmaceutical Sciences and U.S. Pharmacopeia: National Formulary, Mack Publishing Company, Easton, Pa. (1984); Hardman, et al. (2001) Goodman and Gilman's The Pharmacological Basis of Therapeutics, McGraw-Hill, New York, N.Y.; Gennaro (2000) Remington: The Science and Practice of Pharmacy, Lippincott, Williams, and Wilkins, New York, N.Y.; Avis, et al. (eds.) (1993) Pharmaceutical Dosage Forms: Parenteral Medications, Marcel Dekker, NY; Lieberman, et al. (eds.) (1990) Pharmaceutical Dosage Forms: Tablets, Marcel Dekker, NY; Lieberman, et al. (eds.) (1990) Pharmaceutical Dosage Forms: Disperse Systems, Marcel Dekker, N.Y.; Weiner and Kotkoskie (2000) Excipient Toxicity and Safety, Marcel Dekker, Inc., New York, N.Y. In an embodiment of the invention, the pharmaceutical formulation is sterile. Such compositions are part of the present invention.

Pharmaceutical formulations of the present invention include a VEGF trap or VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) and a pharmaceutically acceptable carrier including, for example, water, buffering agents, preservatives and/or detergents.

The scope of the present invention includes desiccated, e.g., freeze-dried, compositions comprising a VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) or a pharmaceutical formulation thereof that includes a pharmaceutically acceptable carrier but substantially lacks water.

In a further embodiment of the invention, a further therapeutic agent that is administered to a subject in association with a VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) disclosed herein is administered to the subject in accordance with the Physicians' Desk Reference 2003 (Thomson Healthcare; 57th edition (Nov. 1, 2002)).

The present invention provides a vessel (e.g., a plastic or glass vial, e.g., with a cap or a chromatography column, hollow bore needle or a syringe cylinder) comprising any of the VEGF traps or VEGF mini-traps (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) or a pharmaceutical formulation comprising a pharmaceutically acceptable carrier thereof. The present invention also provides an injection device comprising a VEGF trap or VEGF mini-trap or formulation set forth herein, e.g., a syringe, a pre-filled syringe or an autoinjector. In an embodiment of the invention, a vessel is tinted (e.g., brown or green) to block out light.

The present invention includes combinations including a VEGF trap or VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) in association with one or more further therapeutic agents. The VEGF trap or VEGF mini-trap and the further therapeutic agent can be in a single composition or in separate compositions. For example, in an embodiment of the invention, the further therapeutic agent is an oligonucleotide (e.g., an oligonucleotide that reduces expression of VEGF, for example, an anti-sense oligonucleotide), Ang-2 inhibitor (e.g., nesvacumab), a Tie-2 receptor activator, an anti-PDGF antibody or antigen-binding fragment thereof, an anti-PDGF receptor or PDGF receptor beta antibody or antigen-binding fragment thereof and/or an additional VEGF antagonist such as aflibercept, conbercept, bevacizumab, ranibizumab, an anti-VEGF aptamer such as pegaptanib (e.g., pegaptanib sodium), a single chain (e.g., VL-VH) anti-VEGF antibody such as brolucizumab, an anti-VEGF DARPin such as the Abicipar Pegol DARPin, a bispecific anti-VEGF antibody, e.g., which also binds to ANG2, such as RG7716, or a soluble form of human vascular endothelial growth factor receptor-3 (VEGFR-3) comprising extracellular domains 1-3, expressed as an Fc-fusion protein.

### Administration and Treatment

The present invention provides methods for treating or preventing a cancer (e.g., whose growth and/or metastasis is mediated, at least in part, by VEGF, e.g., VEGF-mediated angiogenesis) or an angiogenic eye disorder, in a subject, comprising administering a therapeutically effective amount of VEGF trap or VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) to the subject.

The present invention also provides a method for treating cancer (e.g., whose growth and/or metastasis is mediated, at least in part, by VEGF, e.g., VEGF-mediated angiogenesis) or an angiogenic eye disorder in a subject in need thereof, the method comprising administering a therapeutically effective amount of VEGF traps or VEGF mini-trap set forth herein, and optionally a further therapeutic agent, to the body of the subject, e.g., into an eye of the subject. The expression “angiogenic eye disorder,” as used herein, means any disease of the eye which is caused by or associated with the growth or proliferation of blood vessels or by blood vessel leakage. In an embodiment of the invention, administration is done by intravitreal injection. Non-limiting examples of angiogenic eye disorders that are treatable or preventable using the methods herein, include:
age-related macular degeneration (AMD) (e.g., wet or dry, preferably wet),
macular edema,
macular edema following retinal vein occlusion,
retinal vein occlusion (RVO),
central retinal vein occlusion (CRVO),
branch retinal vein occlusion (BRVO),
diabetic macular edema (DME),
choroidal neovascularization (CNV),
iris neovascularization,
neovascular glaucoma,
post-surgical fibrosis in glaucoma,
proliferative vitreoretinopathy (PVR),
optic disc neovascularization,
corneal neovascularization,
retinal neovascularization,
vitreal neovascularization,
pannus,
pterygium,
vascular retinopathy,
diabetic retinopathy in a subject with diabetic macular edema; and
diabetic retinopathies (e.g., non-proliferative diabetic retinopathy (e.g., characterized by a Diabetic Retinopathy Severity Scale (DRSS) level of about 47 or 53) or proliferative diabetic retinopathy; e.g., in a subject that does not suffer from DME or center-involved DME).

The term “treat” or “treatment” refers to a therapeutic measure that reverses, stabilizes or eliminates an undesired disease or disorder (e.g., an angiogenic eye disorder or cancer), for example, by causing the regression, stabilization or elimination of one or more symptoms or indicia of such disease or disorder by any clinically measurable degree, e.g., with regard to an angiogenic eye disorder, by causing a reduction in or maintenance of diabetic retinopathy severity score (DRSS), by improving or maintaining vision (e.g., in best corrected visual acuity e.g., as measured by an increase in ETDRS letters), increasing or maintaining visual field and/or reducing or maintaining central retinal thickness and, with respect to cancer, stopping or reversing the growth, survival and/or metastasis of cancer cells in the subject. Typically, the therapeutic measure is administration of one or more doses of a therapeutically effective amount of VEGF trap or VEGF mini-trap to the subject with the disease or disorder.

An effective or therapeutically effective amount of VEGF trap or VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) for treating or preventing cancer (e.g., which is mediated, at least in part, by angiogenesis) or an angiogenic eye disorder refers to the amount of the VEGF trap or VEGF mini-trap sufficient to cause the regression, stabilization or elimination of the cancer or angiogenic eye disorder, e.g., by regressing, stabilizing or eliminating one or more symptoms or indicia of the cancer or angiogenic eye disorder by any clinically measurable degree, e.g., with regard to an angiogenic eye disorder, by causing a reduction in or maintenance of diabetic retinopathy severity score (DRSS), by improving or maintaining vision (e.g., in best corrected visual acuity e.g., as measured by an increase in ETDRS letters), increasing or maintaining visual field and/or reducing or maintaining central retinal thickness and, with respect to cancer, stopping or reversing the growth, survival and/or metastasis of cancer cells in the subject. In an embodiment of the invention, an effective or therapeutically effective amount of VEGF trap or VEGF mini-trap for treating or preventing an angiogenic eye disorder is about 0.5-25 mg, e.g., in no more than about 100 μl. The amount may vary depending upon the age and the size of a subject to be administered, target disease, conditions, route of administration, and the like. In certain embodiments, the initial dose may be followed by administration of a second or a plurality of subsequent doses of VEGF trap or VEGF mini-trap in an amount that can be approximately the same or less or more than that of the initial dose, wherein the subsequent doses are separated by about 1 to about 8 weeks.

The mode of administration of a VEGF trap or VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) or composition thereof can vary. Routes of administration include parenteral, non-parenteral, oral, rectal, transmucosal, intestinal, intramuscular, subcutaneous, intradermal, intramedullary, intrathecal, direct intraventricular, intravenous, intraperitoneal, intranasal, intraocular, inhalation, insufflation, topical, cutaneous, intraocular, intravitreal, transdermal or intra-arterial.

The present invention provides methods for administering a VEGF trap or VEGF mini-trap (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515) to a subject, comprising introducing the VEGF trap or VEGF mini-trap or a pharmaceutical formulation thereof into the body of the subject. For example, in an embodiment of the invention, the method comprises piercing the body of the subject, e.g., with a needle of a syringe, and injecting the VEGF trap or VEGF mini-trap or a pharmaceutical formulation thereof into the body of the subject, e.g., into the eye, vein, artery, muscular tissue or subcutis of the subject.

In an embodiment of the invention, intravitreal injection of a pharmaceutical formulation of the present invention (which includes a VEGF trap or VEGF mini-trap of the present invention (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515)) includes the step of piercing the eye with a syringe and needle (e.g., 30-gauge injection needle) containing the formulation and injecting the formulation (e.g., less than or equal to about 100 microliters) into the vitreous of the eye (e.g., with a sufficient volume as to deliver a therapeutically effective amount as set forth herein, e.g., of about 0.5-20 mg VEGF mini-trap). Optionally, the method includes the steps of administering a local anesthetic (e.g., proparacaine, lidocaine or tetracaine), an antibiotic (e.g., a fluoroquinolone), antiseptic (e.g., povidone-iodine) and/or a pupil dilating agent to the eye being injected. In an embodiment of the invention, a sterile field around the eye to be injected is established before the injection. In an embodiment of the invention, following intravitreal injection, the subject is monitored for elevations in intraocular pressure, inflammation and/or blood pressure.

The term “in association with” indicates that components, a VEGF trap or VEGF mini-trap of the present invention (e.g., REGN10104; mREGN10104; REGN10102; REGN10105; REGN10117; REGN10103; REGN10187; mREGN10187; REGN10511; mREGN10511; REGN10512; mREGN10512; REGN10513; mREGN10513; REGN10514; REGN11095; REGN10515; or mREGN10515), along with another agent such as anti-ANG2, can be formulated into a single composition, e.g., for simultaneous delivery, or formulated separately into two or more compositions (e.g., a kit including each component). Components administered in association with each another can be administered to a subject at a different time than when the other component is administered; for example, each administration may be given non-simultaneously (e.g., separately or sequentially) at intervals over a given period of time. Separate components administered in association with each another may also be administered essentially simultaneously (e.g., at precisely the same time or separated by a non-clinically significant time period) during the same administration session. Moreover, the separate components administered in association with each another may be administered to a subject by the same or by a different route

As used herein, the term “subject” refers to a mammal (e.g., rat, mouse, cat, dog, cow, sheep, horse, goat, rabbit), preferably a human, for example, in need of prevention and/or treatment of a cancer or an angiogenic eye disorder. The subject may have cancer or angiogenic eye disorder or be predisposed to developing cancer or angiogenic eye disorder.

In an embodiment of the invention, any method including the step of intravitreally injecting a VEGF trap or VEGF mini-trap of the present invention, e.g., for treating or preventing an angiogenic eye disorder, does not lead to a significant increase in intraocular pressure and/or blood pressure.

### EXAMPLES

These examples are intended to exemplify the present invention are not a limitation thereof. Compositions and methods set forth in the Examples form part of the present invention.

### Example 1: Binding Kinetic Analysis of VEGF Traps and VEGF Mini-Traps and VEGF on Receptor Captured Surface

The binding characteristics of various VEGF traps and VEGF mini-traps was evaluated.

TABLE 1-1
VEGF traps and VEGF mini-traps

| REGN# | VEGF trap or mini-trap |
|---|---|
| REGN110 | Human VEGF₁₆₅ |
| REGN3 | VEGF Trap hIgG1 Fc |
| | (aflibercept) |
| REGN7483 | VEGF mini-trap hIgG1 Fc |
| REGN10104 | VEGF Trap hIgG1 Stealth Fc |
| mREGN10104 | VEGF mini-trap hIgG1 Stealth Fc |
| REGN10102 | VEGF Trap hIgG2 Fc w_2 Cys |
| REGN10105 | VEGF mini-trap hIgG2 Fc w_2 Cys |
| REGN10117 | VEGF Trap hIgG2 Fc w_4 Cys |
| REGN10103 | VEGF mini-trap hIgG2 Fc w_4 Cys |
| REGN10187 | VEGF Trap hIgG4 Stealth Fc |
| mREGN10187 | VEGF mini-trap hIgG4 Stealth Fc |
| REGN10514 | VEGF Trap hIgG1 Mut GGGL (SEQ |
| | ID NO: 94) Fc |
| REGN11095 | VEGF mini-trap hIgG1 Mut GGGL |
| | (SEQ ID NO: 94) Fc |

REGN3 =
(SEQ ID NO: 69)
SDTGRPFVEMYSEIPEIIHMTEGRELVIPCRVTSPNITVTLKKFPLDTL
IPDGKRIIWDSRKGFIISNATYKEIGLLTCEATVNGHLYKTNYLTHRQT
NTIIDVVLSPSHGIELSVGEKLVLNCTARTELNVGIDFNWEYPSSKHQH
KKLVNRDLKTQSGSEMKKFLSTLTIDGVTRSDQGLYTCAASSGLMTKKN
STFVRVHEKDKTHTCPPC240PAPELLGGPSVFLFPPKPKDTLMISRTP
EVTCVVVDVSHEDPEVKFNWYVDGVEVHNAKTKPREEQYNSTYRVVSVL
TVLHQDWLNGKEYKCKVSNKALPAPIEKTISKAKGQPREPQVYTLPPSR
DELTKNQVSLTCLVKGFYPSDIAVEWESNGQPENNYK403TTPPVLDSD
GSFFLYSKLTVDKSRWQQGNVESCSVMHEALHNHYTQKSLSLSPGK

### Experimental Procedure (Include Description of Relevant Cell Lines, Proteins, Reagents, and Instrument Type and Model).

Equilibrium dissociation constants (K_{D} values) for human VEGF₁₆₅ binding to various purified VEGF mini-trap constructs were determined using a real-time surface plasmon resonance biosensor using a Biacore 3000 instrument. All binding studies were performed in 10 mM HEPES, 150 mM NaCl, 3 mM EDTA, and 0.05% v/v Surfactant Tween-20, pH 7.4 (HBS-ET) running buffer at 25° C. The Biacore sensor surface was first derivatized by amine coupling with a monoclonal mouse anti-VEGFR1 antibody (REGN18) to capture VEGF mini-trap constructs. Binding studies were performed on the following human VEGF reagents: human VEGF₁₆₅. Different concentrations of VEGF₁₆₅ reagent were prepared in HBS-ET running buffer (2.5 nM-0.078 pM; 2-fold serial dilution for human VEGF₁₆₅) and then injected over anti-VEGFR1 captured VEGF mini-trap constructs surface for 108 seconds at a flow rate of 90 μL/minute. Dissociation of bound VEGF₁₆₅ reagent from VEGF mini-trap constructs was monitored for 60 minutes in HBS-ET running buffer. Kinetic association (ka) and dissociation (kd) rate constants were determined by fitting the real-time sensorgrams to a 1:1 binding model using Scrubber 2.0c curve fitting software. Binding dissociation equilibrium constants (K_{D}) and dissociative half-lives (t½) were calculated from the kinetic rate constants as:

$${K}_{D} ( M ) = \frac{kd}{k 2} , and t \frac{1}{2} ( min ) = \frac{ln ( 2 )}{60 * kd}$$

The binding kinetic parameters for human VEGF165 binding to different VEGF mini-trap constructs at 25° C. are shown in Table 1-2 and Table 1-3. All VEGF mini-traps showed comparable binding kinetics as aflibercept REGN3.

TABLE 1-2
Binding Kinetic Parameters of Human VEGF₁₆₅ Binding to Different VEGF
Mini-Trap Constructs at 25° C.

| | | | Trap/ | | | | | |
|---|---|---|---|---|---|---|---|---|
| VEGF | | | mini-trap | 2.5 nM | | | | |
| Trap or | | | Capture | hVEGF₁₆₅ | | | | |
| mini- | | Human | Level | Bound | | | | |
| trap | REGN# | IgG | (RU) | (RU) | ka(1/Ms) | kd(1/s) | K_{D}(M) | t½(min) |
| Trap | REGN3 | IgG1 | 15.8 ± 0.1 | 4.7 | 9.70E+06 | 1.70E−05 | 1.80E−12 | 679 |
| mini-trap | REGN7483 | IgG1 | 17.0 ± 0.1 | 9.37 | 1.01E+07 | 1.95E−05 | 1.90E−12 | 591 |
| Trap | REGN10104 | IgG1 | 21.1 ± 0.2 | 7.3 | 9.20E+06 | 1.01E−05 | 1.10E−12 | 1149 |
| | | stealth | | | | | | |
| mini- | mREGN10104 | IgG1 | 20.7 ± 0.1 | 12.2 | 9.50E+06 | 1.70E−05 | 1.78E−12 | 681 |
| trap | | stealth | | | | | | |
| Trap | REGN10102 | IgG2 | 18.6 ± 0.2 | 6.6 | 1.06E+07 | 2.77E−05 | 2.60E−12 | 417 |
| | | w_2cys | | | | | | |
| mini- | REGN10105 | IgG2 | 19.7 ± 0.2 | 10.5 | 7.62E+06 | 1.44E−05 | 1.89E−12 | 801 |
| trap | | w_2cys | | | | | | |
| Trap | REGN10117 | IgG2 | 14.7 ± 0.1 | 4.2 | 7.30E+06 | ≤1e−5 | 1.37E−12 | ≥1155 |
| | | w_4cys | | | | | | |
| mini- | REGN10103 | IgG2 | 23.9 ± 0.3 | 14.8 | 9.30E+06 | ≤1e−5 | 1.08E−12 | ≥1155 |
| trap | | w_4cys | | | | | | |
| Trap | REGN10187 | IgG4 | 26.4 ± 0.3 | 10.5 | 9.50E+06 | 1.73E−05 | 1.82E−12 | 668 |
| | | stealth | | | | | | |
| mini- | mREGN10187 | IgG4 | 25.1 ± 0.3 | 14.3 | 7.30E+06 | ≤1e−5 | 1.37E−12 | ≥1155 |
| trap | | stealth | | | | | | |
| Trap | REGN10514 | hIgG1- | | | | | | |
| | | GGGL | | | | | | |
| | | (SEQ ID | | | | | | |
| | | NO: 94) | | | | | | |
| mini- | REGN11095 | hIgG1- | | | | | | |
| trap | | GGGL | | | | | | |
| | | (SEQ ID | | | | | | |
| | | NO: 94) | | | | | | |

TABLE 1-3
Binding Kinetic Parameters of Human VEGF₁₆₅ Binding to Different VEGF
Mini-Trap Constructs at 25° C. (Additional Measurements) (“GGGL” disclosed as SEQ
ID NO: 94 and “CPPAPAPPVA” disclosed as SEQ ID NO: 106)

| | | VEGF | 2 nM | | | | |
|---|---|---|---|---|---|---|---|
| | | Trap | VEGF₁₆₅ | | | | |
| | | captured | Bound | ka | kd | KD | t½ |
| VEGF Trap/mini-trap | Construct | (RU) | (RU) | (1/Ms) | (1/s) | (M) | (min) |
| REGN11095 | VEGF mini Trap hIgG1 | 30 + 0.5 | 19.4 | 1.16E+07 | ≤1e−5 | 8.60E−13 | ≥1155 |
| REGN11095 (Alexa 488) | Mut GGGL (cleaved) | 24 + 0.2 | 14.9 | 1.16E+07 | ≤1e−5 | 8.65E−13 | ≥1155 |
| REGN10105 | VEGF mini Trap hIgG2 Fc w_2 cys | 29 + 0.2 | 20.4 | 9.30E+06 | ≤1e−5 | 1.08E−12 | ≥1155 |
| REGN10105 (Alexa 488) | CPPAPAPPVA (cleaved) | 24 + 0.1 | 18.0 | 1.36E+07 | ≤1e−5 | 7.40E−13 | ≥1155 |
| REGN3 | VEGF-Trap | 32 + 0.6 | 12.0 | 9.98E+06 | ≤1e−5 | 1.00E−12 | ≥1155 |

### Example 2: Evaluation of the Ability of VEGF Mini-Traps to Block the Activation of VEGFR1 and VEFGR2 by VEGF₁₆₅ or VEGF₁₂₁ in a Luciferase Bioassay

The ability of various VEGF traps and VEGF mini-traps to block VEGFR activation was evaluated.

TABLE 2-1
VEGF Traps and VEGF Mini-Traps

| REGN# | Description |
|---|---|
| | Human VEGF₁₂₁ |
| REGN110 | Human VEGF₁₆₅ |
| REGN3 | VEGF Trap hIgG1 Fc |
| | (aflibercept) |
| REGN7483 | VEGF mini-trap hIgG1 Fc |
| REGN10104 | VEGF Trap hIgG1 Fc Stealth |
| mREGN10104 | VEGF mini-trap hIgG1 Fc |
| | Stealth |
| REGN10102 | VEGF Trap hIgG2 Fc w_2 Cys |
| REGN10105 | VEGF mini-trap hIgG2 Fc w_2 |
| | Cys |
| REGN10117 | VEGF Trap hIgG2 Fc w_4 Cys |
| REGN10103 | VEGF mini-trap hIgG2 Fc w_4 |
| | Cys |
| REGN10187 | VEGF Trap hIgG4 Stealth Fc |
| mREGN10187 | VEGF mini-trap hIgG4 Stealth |
| | Fc |
| REGN10514 | VEGF Trap hIgG1 Mut GGGL |
| | (SEQ ID NO: 94) Fc |
| REGN11095 | VEGF mini-trap hIgG1 Mut |
| | GGGL (SEQ ID NO: 94) Fc |

### Experimental Procedure (Include Description of Relevant Cell Lines, Proteins, Reagents, and Instrument Type and Model).

### Cell Line

Two cell lines were generated to measure the ligand signaling pathway through VEGFR1 and VEGFR2 respectively. For VEGFR1, HEK293/D9/Flt (1-7)-IL18Rα/Flt-IL18Rβ clone V3H9 was constructed by two chimeric receptors incorporating the VEGFR1 extracellular domain Flt1 (1-7) fused to the cytoplasmic domain of either IL18Rα or IL18Rβ. The chimeric receptors were transfected into a cell line with an integrated NFκB-luciferase-IRES-eGFP reporter gene. The extracellular VEGFR1 is dimerized upon binding VEGF, resulting in interaction of the IL18Rα and IL18Rβ intracellular domains, NFκB signaling, and subsequent luciferase production. Similarly, for VEGFR2, HEK293/D9/Flk (1-7)-IL18Rα/Flt-IL18Rβ was constructed by two chimeric receptors incorporating the VEGFR2 extracellular domain Flk1 (1-7) fused to the cytoplasmic domain of either IL18Rα or IL18Rβ.

### Assay Procedure

HEK293/D9/Flt-IL18Rα/Flt-IL18Rβ (VEGFR1) or HEK293/D9/Flk(1-7)-IL18Rα/Flt-IL18Rβ (VEGFR2) cells were plated in 96-well white opaque plates (Nunc, Cat #136101) at 10,000 cells/well in OptiMEM (INVITROGEN, Cat #31985) with 0.5% FBS (Seradigm, Cat #1500-500) and incubated at 37° C., 5% CO₂ overnight. The next day, cells were differentially treated with a 1:3 serial dilution of VEGF Trap or Mini Trap proteins ranging in concentration from 5000 pM to 0.085 pM, followed by the addition of a fixed concentration of VEGF₁₂₁ (R&D SYSTEMS Cat #4644-VS) or VEGF₁₆₅ (REGN110) ligand protein at 40 μM and incubated for 6 hours at 37° C., 5% CO₂. One-Glo luciferase substrate (PROMEGA, Cat #E6130) was then added to the cells and luminescence was measured using a VICTOR™ X5 Multilabel plate reader (PerkinElmer, Model 2030-0050). Data were analyzed using a 4-parameter logistic equation over an 11-point response curve with GraphPad Prism software to determine EC₅₀ and IC₅₀ values.

### Results Summary and Conclusions

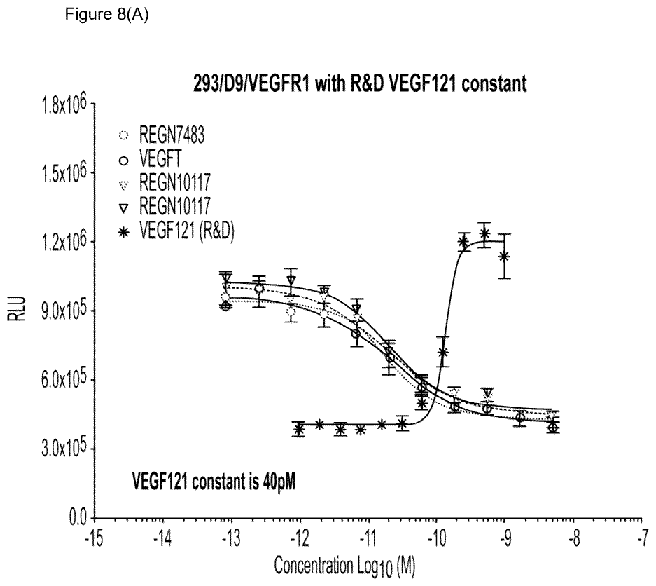

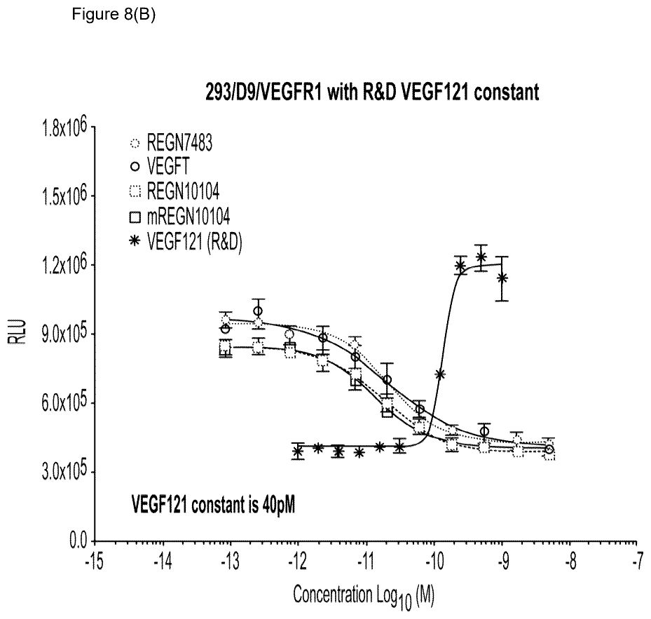

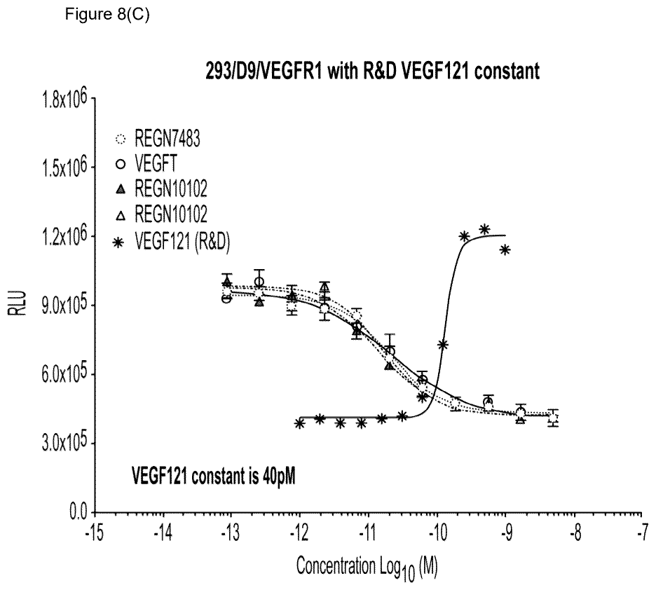

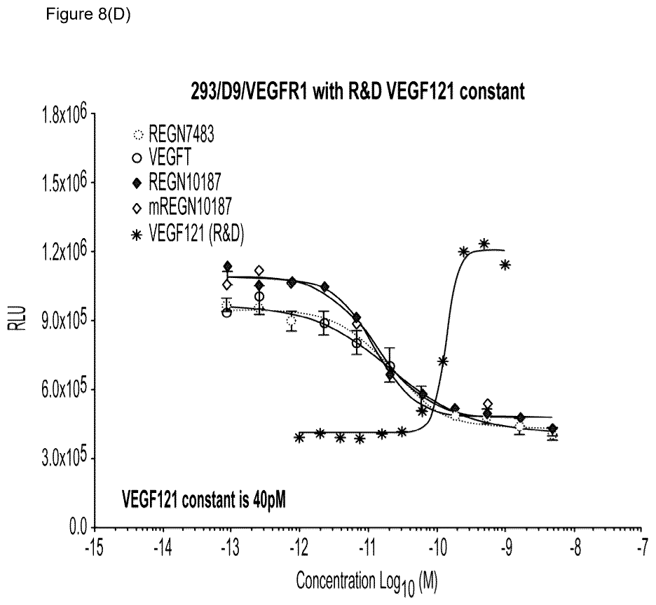

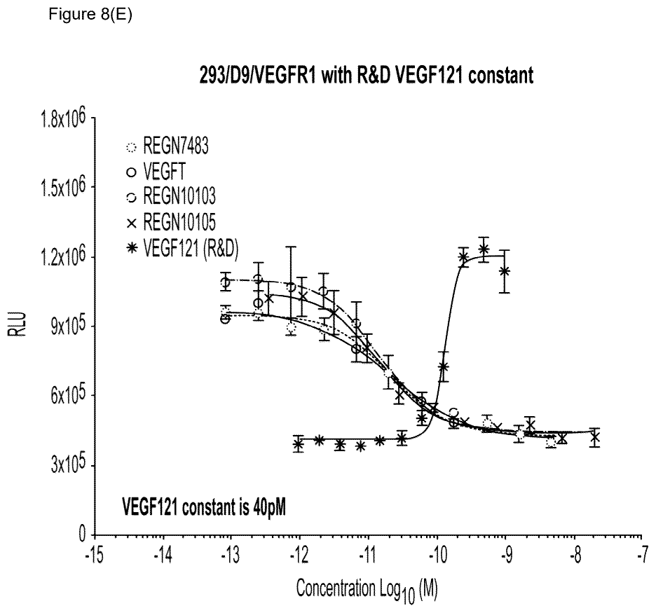

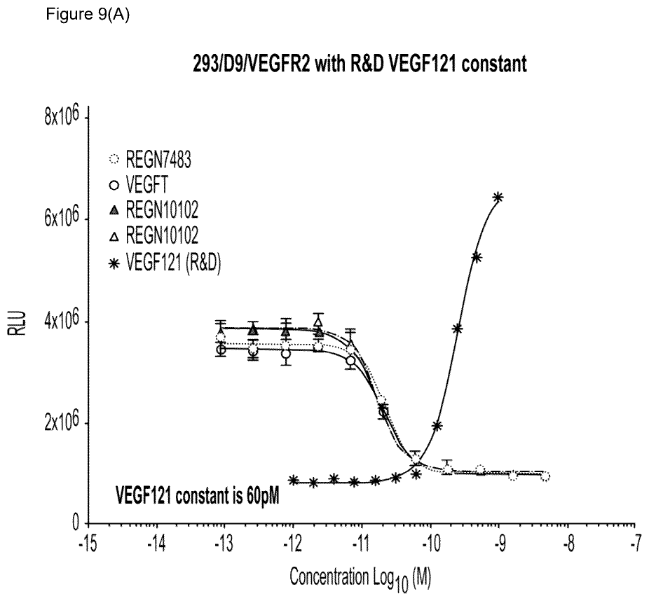

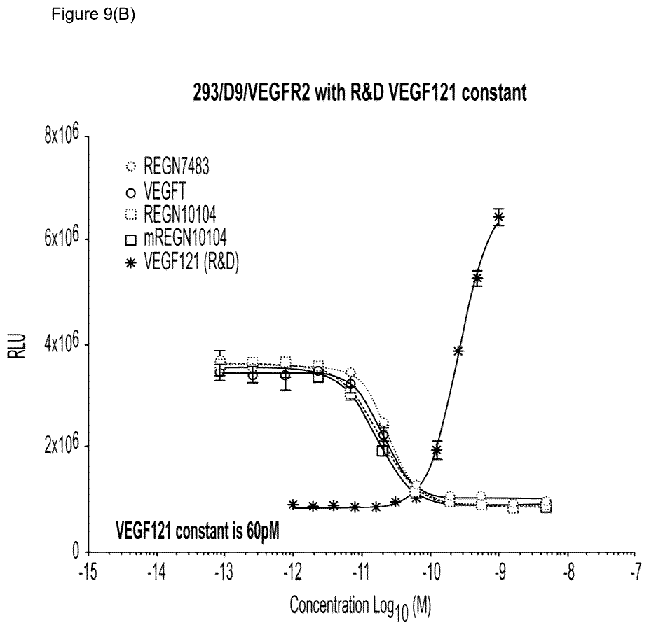

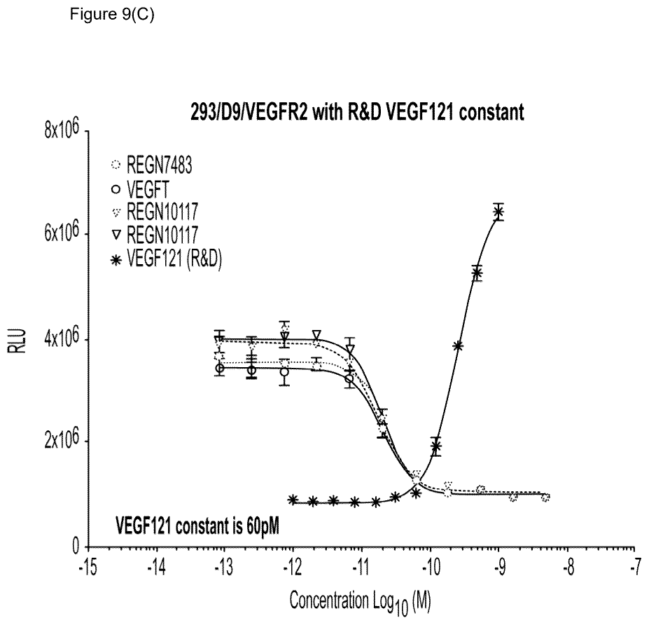

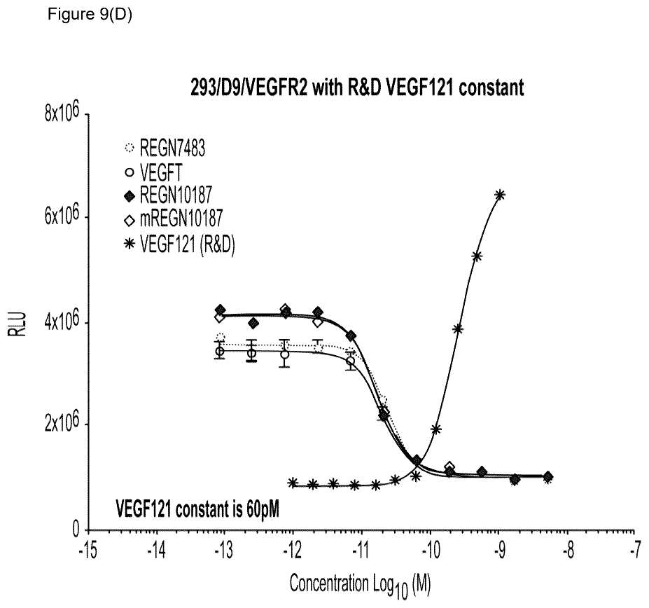

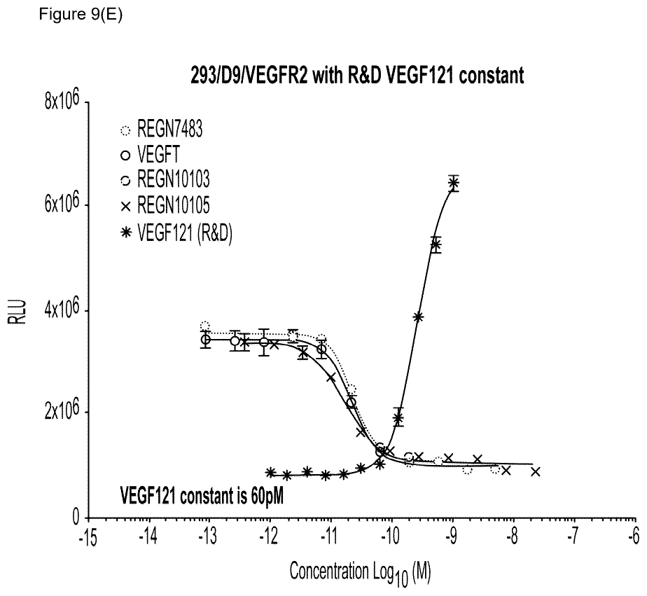

VEGF₁₂₁ activates HEK293/D9/Flt-IL18Rα/Flt-IL18Rβ (VEGFR1) or HEK293/D9/Flk (1-7)-IL18Rα/Flt-IL18Rβ (VEGFR2) cells with EC₅₀ values of ˜30 pM (FIG. 8 and FIG. 9 and Table 2-2).

All VEGF mini-traps inhibited 20 pM of VEGF₁₂₁ mediated activation of VEGFR1 or VEGFR2 with similar IC₅₀ values to that observed with full length VEGF trap REGN3 (FIG. 8 and FIG. 9 and Table 2-2 and Table 2-3).

TABLE 2-2
Bioassay Activity of VEGF mini-traps
Blocking VEGF₁₂₁ or VEGF₁₆₅
in VEGFR1 and VEGFR2 Cells

| | | | Inhibition | | Inhibition | |
|---|---|---|---|---|---|---|
| | | | of 20 pM | | of 20 pM | |
| VEGF | | | VEGF₁₂₁ | | VEGF₁₆₅ | |
| Trap | | | IC50 (M) | | IC50 (M) | |
| or | | | VEG | VEG | VEG | VEG |
| mini- | | Human | FR1 | FR2 | FR1 | FR2 |
| trap | REGN# | IgG | assay | assay | assay | assay |
| Trap | REGN | IgG1 | 2.15 | 2.14 | 3.10 | 3.21 |
| | 3 | | E−11 | E−11 | E−11 | E−11 |
| mini- | REGN | IgG1 | 2.09 | 2.37 | 3.36 | 4.03 |
| trap | 7483 | | E−11 | E−11 | E−11 | E−11 |
| Trap | REGN | IgG1 | 1.91 | 1.77 | | |
| | 10104 | stealth | E−11 | E−11 | | |
| mini- | mREGN | IgG1 | 1.34 | 1.60 | | |
| trap | 10104 | stealth | E−11 | E−11 | | |
| Trap | REGN | IgG2 | 1.43 | 1.72 | | |
| | 10102 | w_2cys | E−11 | E−11 | | |
| mini- | REGN | IgG2 | 1.68 | 1.79 | | |
| trap | 10105 | w_2cys | E−11 | E−11 | | |
| Trap | REGN | IgG2 | 2.02 | 1.82 | | |
| | 10117 | w_4cys | E−11 | E−11 | | |
| mini- | REGN | IgG2 | 2.11 | 2.02 | | |
| trap | 10103 | w_4cys | E−11 | E−11 | | |
| Trap | REGN | IgG4 | 1.25 | 1.64 | | |
| | 10187 | stealth | E−11 | E−11 | | |
| mini- | mREGN | IgG4 | 1.32 | 1.63 | | |
| trap | 10187 | stealth | E−11 | E−11 | | |
| Trap | REGN | hIgG1- | | | | |
| | 10514 | GGGL | | | | |
| | | (SEQ ID | | | | |
| | | NO: | | | | |
| | | 94) | | | | |
| mini- | REGN | hIgG1- | | | | |
| trap | 11095 | GGGL | | | | |
| | | (SEQ ID | | | | |
| | | NO: | | | | |
| | | 94) | | | | |

TABLE 2-3
Bioassay Activity of VEGF mini-traps Blocking VEGF₁₂₁ or VEGF₁₆₅ in
VEGFR1 and VEGFR2 Cells (Additional Measurements)
IC50 Table (M)

| | VEGFR1 40 pM | | VEGFR2 20 pM | |
|---|---|---|---|---|
| | VEGF165 | VEGFR1 40 pM | VEGF165 | VEGFR2 60 pM |
| | (REGN110) | R&D VEGF121 | (REGN110) | R&D VEGF121 |
| REGN3 | 1.49E−11 | 1.12E−11 | 2.16E−11 | 1.52E−11 |
| REGN10105 | 1.06E−11 | 1.74E−11 | 2.46E−11 | 1.80E−11 |
| REGN11095 | 2.19E−11 | 2.16E−11 | 2.39E−11 | 2.30E−11 |
| IgG1 Isotype | ND | ND | ND | ND |

### Example 3: Determine Relative Cleavage Efficiency of VEGF-Trap Variants Mediated by Ides Protease Using SDS-PAGE Analysis

The ability of various VEGF traps to be cleaved by IdeS protease was determined.

TABLE 3-1
VEGF Traps and VEGF Mini-Traps

| REGN# | Construct Details | |
|---|---|---|
| REGN3 | Full-length | VEGF trap hIgG1 |
| | VEGF Trap, | isotype |
| | aflibercept | |
| REGN10102 | Full-length | VEGF trap hIgG2 |
| | VEGF trap | Fc w_2 cys |
| REGN10511 | Full-length | VEGF trap hIgG1- |
| | VEGF trap | 2 Chimera Fc |
| REGN10512 | Full-length | VEGF trap hIgG1 |
| | VEGF trap | Mut GGGF (SEQ |
| | | ID NO: 92) Fc |
| REGN10514 | Full-length | VEGF trap hIgG1 |
| | VEGF trap | Mut GGGL (SEQ |
| | | ID NO: 94) Fc |
| REGN10515 | Full-length | VEGF trap hIgG1 |
| | VEGF trap | Del (APE) Fc |

### Experimental Procedure (Include Description of Relevant Cell Lines, Proteins, Reagents, and Instrument Type and Model)

Relative cleavage efficiency of various VEGF trap constructs mediated by Ides protease was determined using SDS-PAGE analysis. All cleavage studies were performed in 10 mM sodium phosphate, 40 mM sodium chloride, 0.03% polysorbate 20, and 5% sucrose, pH 6.2 buffer at room temperature. The various VEGF trap constructs were first normalized to 0.1 mg/ml at a scale of 0.1 mg. For each VEGF trap construct, 100 μL sample aliquots were prepared from the normalized stock in replicates of 5. Stock Ides protease (0.1 mg/mL) was then added to all samples at a ratio of 5 ng protease/ug VEGF trap. Samples were allowed to incubate for periods of 0, 1, 2, 4, and 24 hr. Cleavage was stopped at each time point by addition of 0.1 mL of a stock amine based pH2.8 buffer. Sample timepoints collected were grouped by VEGF trap variant type, and 2 ug of each digest sample was then analyzed by SDS-PAGE using a 4-20% Tris-Glycine gel under non-reducing conditions. Each gel was run for a period of 1 hr at constant 300 mA. Simply-Blue Safe-Stain was used for subsequent staining of each gel. The de-stained gels were analyzed visually to ascertain relative cleavage efficiency of each VEGF trap variant over incubation time as mediated by Ides protease. Extent of cleavage efficiency was determined by comparison of relative levels of non-cleaved VEGF trap dimer, partially cleaved VEGF trap dimer, fully cleaved VEGF trap dimer (VEGF mini-trap), and Fc monomer fragment. A ranking of relative cleavage efficiency among the VEGF trap variants was then determined based on extent of Ides mediated cleavage observed.

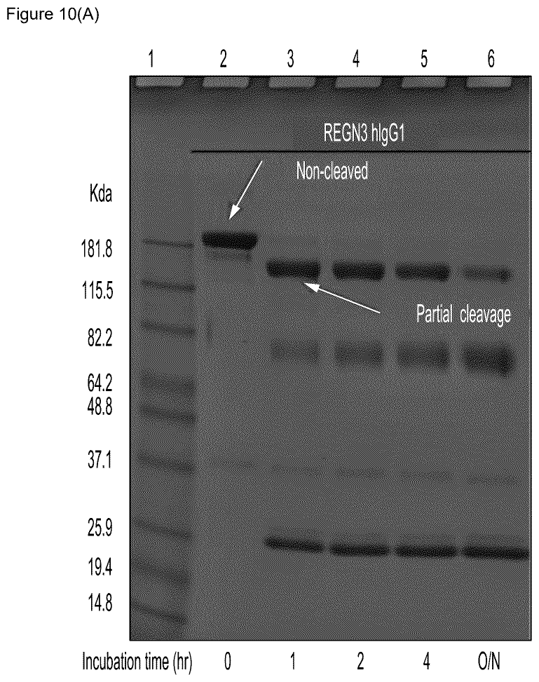

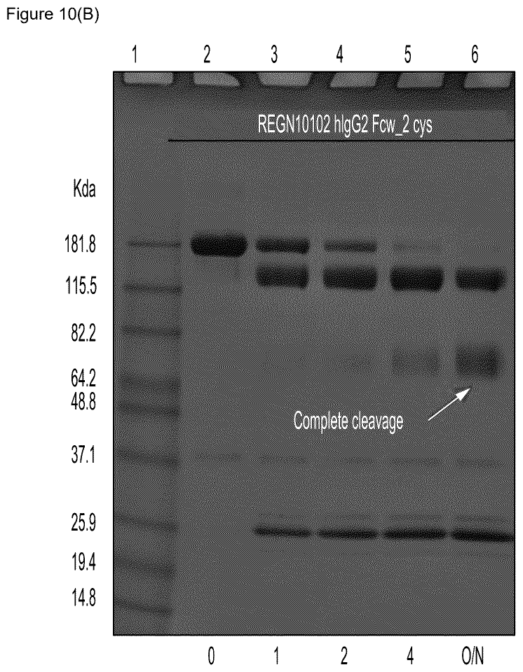

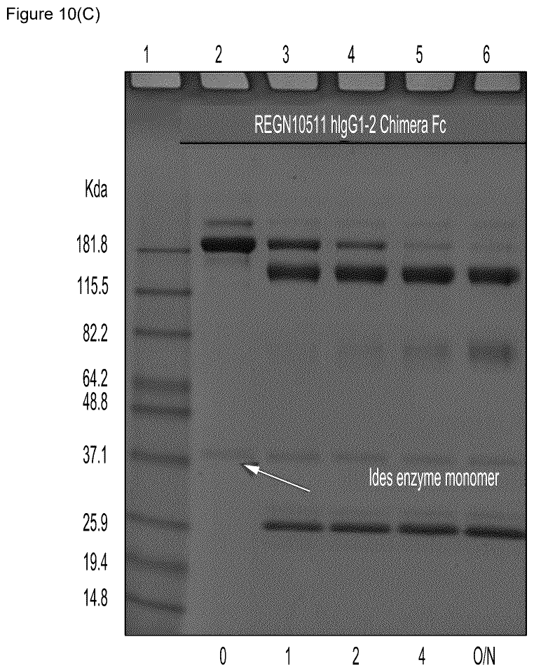

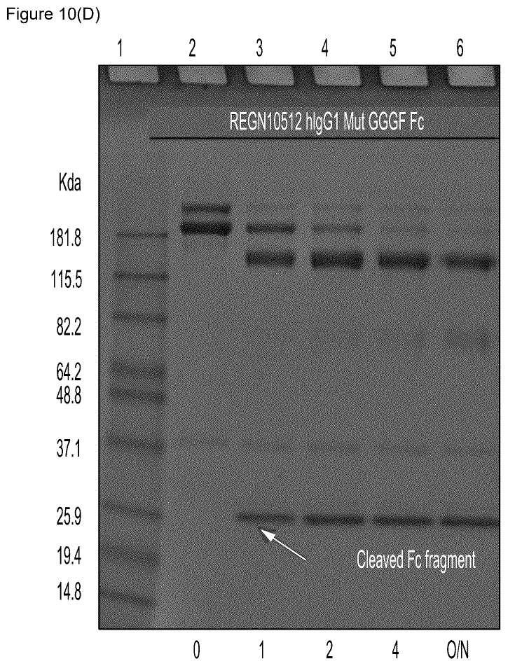

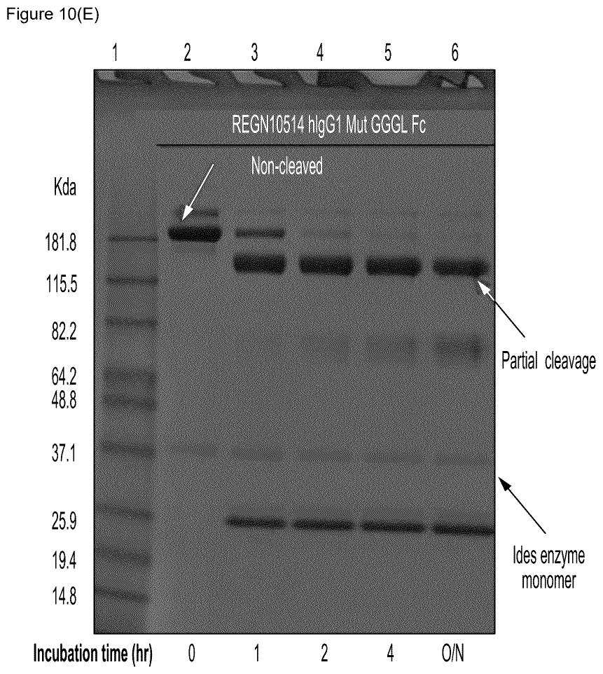

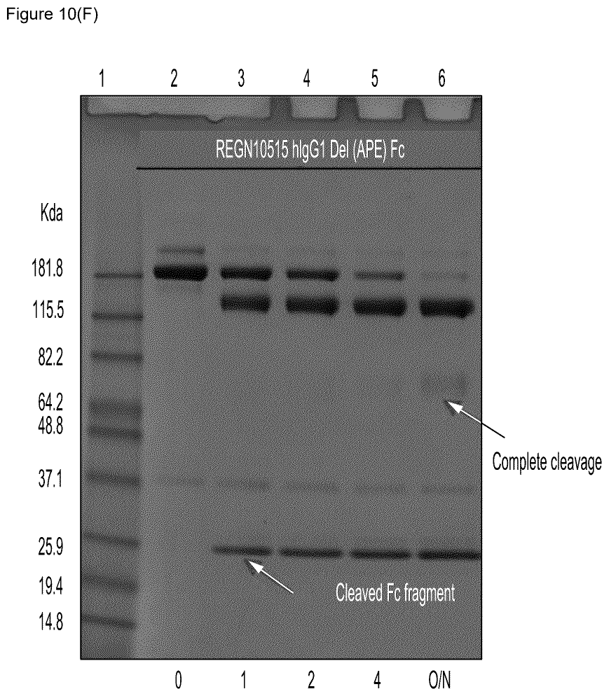

SDS-PAGE analysis of IdeS protease mediated cleavage of each VEGF trap variant tested is shown in FIG. 10 (A-F).

### Example 4: Assay of VEGF Mini-Traps for Reactivity with Pre-Existing Anti-Hinge Antibodies (AHA)

The reactivity of various VEGF mini-traps to pre-existing human and monkey anti-hinge antibodies was determined.

TABLE 3-1
VEGF Mini-Traps

| Mini-Trap | Human IgG | Details |
|---|---|---|
| REGN7483 | IgG1 | Enzymatically cleaved from aflibercept |
| mREGN10104 | IgG1 stealth | Enzymatically cleaved from VEGF trap- |
| | | IgG1s |
| REGN10105 | IgG2 (2Cys) | Recombinantly expressed in CHO |
| REGN10105 | IgG2 (2Cys) | Enzymatically cleaved from VEGF trap- |
| | | IgG2 |
| REGN10103 | IgG2 (4Cys) | Enzymatically cleaved from VEGF trap- |
| | | IgG2 |
| mREGN10187 | IgG4 stealth | Enzymatically cleaved from VEGF trap- |
| | | IgG4s |

### Experimental Procedure (Include Description of Relevant Cell Lines, Proteins, Reagents, and Instrument Type and Model)

The potential immunogenicity of VEGF mini-traps towards pre-existing anti-hinge antibodies (AHA) was evaluated in an Anti-Drug Antibody (ADA) assay. See the illustration of the assay format in FIG. 11(A) This ADA assay is an Electrochemiluminescent Immunoassay (ECL) bridging assay, using Biotin- and Ruthenium-labeled drugs (mini-traps) to form a bridge. Anti-drug antibody binds Biotin- and Ruthenium-labeled drugs (VEGF mini-trap) forming a bridged complex, which is then captured by the streptavidin-coated plate surface. Electric current activates Ruthenium-labeled drug to produce an electrochemiluminescent signal.

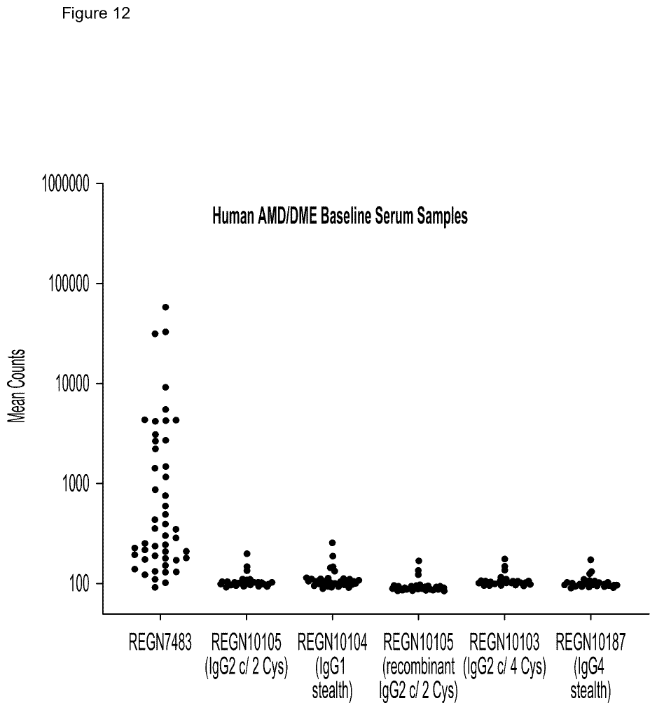

Six VEGF mini-traps were tested in this assay using 48 human AMD/DME baseline serum samples and 24 monkey naïve serum samples. An anti-VEGFR1 monoclonal antibody (REGN18) was used as positive control. Results are summarized in FIG. 11(B) and FIG. 12.

### Results Summary and Conclusions

Data showed that pre-existing anti-hinge antibodies (AHA) in naïve monkey serum samples or human AMD/DME baseline serum samples did not recognize the VEGF mini-trap constructs, except REGN7483 (hIgG1) which showed variable range of counts. Positive control anti-VEGFR1 mAb showed mean counts in the range of 6858-15055.

### Example 5: Size Analysis of In Vitro Complexes Formed Between VEGF Mini-Traps (REGN10105 and REGN11095) and Recombinant Human VEGF₁₆₅ (REGN110) by Size Exclusion Chromatography Coupled to Multi-Angle Laser Light Scattering (SEC-MALS)

The stoichiometry of two VEGF mini-traps (REGN10105 and REGN11095) for binding to human VEGF₁₆₅ (hVEGF₁₆₅; REGN110) was determined.

### Materials and Equipment

TABLE 5-1
Instrumentation List

| Instrumentation | Source |
|---|---|
| Waters Quaternary Solvent manager | Waters Corporation, |
| Waters Column Compartment | Milford, MA |
| Waters Sample Manager | |
| Waters UV detector | |
| Waters ACQUITY UPLC BEH ®200 SEC | |
| column (2.5 μm, 4.6 mm × 300 mm) | |
| microDawn light scattering detector | Wyatt Technology, |
| Optilab ® UT-rEX refractive index detector | Santa Barbara, CA |

TABLE 5-2
Reagent and Materials

| Reagent | Source |
|---|---|
| 10X Dulbecco's Phosphate Buffered Saline | Gibco |
| HPLC grade water | J. T. Baker |
| Sodium Phosphate Dibasic Heptahydrate | J. T. Baker |
| Sodium phosphate monobasic, monohydrate | J. T. Baker |
| Sodium Perchlorate | Sigma Aldrich |

### Methods

SEC-MALS Mobile Phase Buffer. The mobile phase buffer (10 mM sodium phosphate, 500 mM sodium chloride, pH 7.0±0.1) was prepared by combining 1.4 g sodium phosphate monobasic monohydrate, 10.7 g sodium phosphate dibasic heptahydrate, and 500 mL 5 M sodium chloride; the solution was then brought to a volume to 5.0 L with HPLC grade water. The final measured pH of the buffer was 7.0. The mobile phase buffer was filtered (0.2 μm) before use.

SEC-MALS. The SEC-MALS system was composed of an UPLC connected to ultraviolet (UV), light scattering (LS), and refractive index (RI) detectors. The detectors were connected in series in the following order: UV-LS-RI. LS and RI detectors were calibrated according to instructions provided by Wyatt Technology. A BEH®200 SEC column size exclusion column was connected to the SEC-MALS system and pre-equilibrated in 10 mM sodium phosphate, 500 mM sodium chloride, pH 7.0 (SEC mobile phase buffer) with a flow rate of 0.3 mL/min prior to injection of samples. Defined amounts of mini-traps were each combined with hVEGF165 (REGN110) and diluted in 1×DPBS, pH 7.4 to yield different ratios. All samples were incubated at ambient temperature for 2 hours and maintained unfiltered at 4° C. prior to injection. Bovine serum albumin (BSA; 2 mg/mL; 10 μg sample load) was injected separately and included as a system suitability control.

MALS Data Analysis. Data were analyzed using ASTRA V software (version 7.3.1.9, Wyatt Technology). The data were fit to the equation that relates the excess scattered light to the solute concentration and weight-average molar mass, Mw, (Kendrick et al., Online Size-Exclusion High-Performance Liquid Chromatography Light Scattering and Differential Refractometry Methods to Determine Degree of Polymer Conjugation to Proteins and Protein-Protein or Protein-Ligand Association States”. (2001). Anal Biochem. 299(2), 136-46; Wyatt (1993) Anal. Chim. Acta 272(1), 1-40, Light Scattering and the Absolute Characterization of Macromolecules):

$$\frac{K * c}{R ( θ , c )} = \frac{1}{MwP ( θ )} + 2 {A}_{2} C \qquad Equation 1$$

where c is the solute concentration, R(θ,c) is the excess Raleigh ratio from the solute as a function of scattering angle and concentration, Mw is the molar mass, P(θ) describes the angular dependence of scattered light (˜1 for particles with radius of gyration<50 nm), A₂ is the second virial coefficient in the expansion of osmotic pressure (which can be neglected since measurements are performed on dilute solutions) and

$$K * = \frac{4 {π}^{2} {n}_{0}^{2}}{{N}_{A} {λ}_{0}^{4}} {( \frac{dn}{dc} )}^{2} \qquad Equation 2$$

where nₒ represents the solvent refractive index, N_{A} is Avogadro's number, λ₀ is the wavelength of the incident light in a vacuum, and dn/dc represents the specific refractive index increment for the solute.

The molar mass of BSA monomer served to evaluate the calibration constants of the light scattering and differential refractive index detectors during data collection (system suitability check). The relative standard deviation (% RSD) of the average molar mass of BSA determined from the UV and RI detectors was ≤5.0%.

The normalization coefficients for the light scattering detectors, inter-detector delay volume and band broadening terms were calculated from the BSA chromatograms collected for the SEC-MALS condition employed. These values were applied to the data files collected for all the other samples to correct for these terms.

The dn/dc value and the extinction coefficient at 280 nm (corrected for glycosylation) were experimentally determined using the protein conjugate analysis provided in the Astra software. The corrected extinction coefficient and dn/dc value was used to analyze all protein-protein complex samples.

### Results

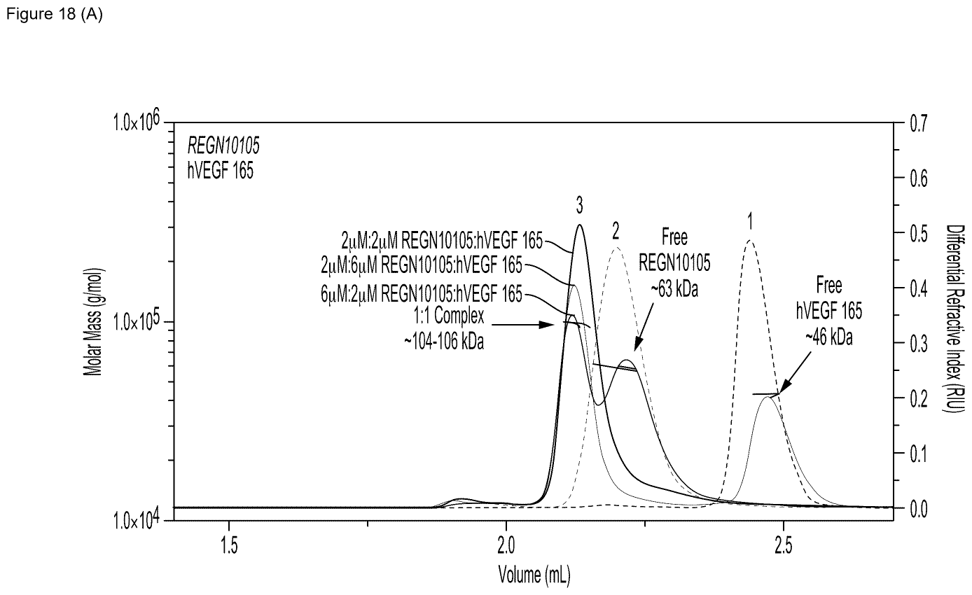

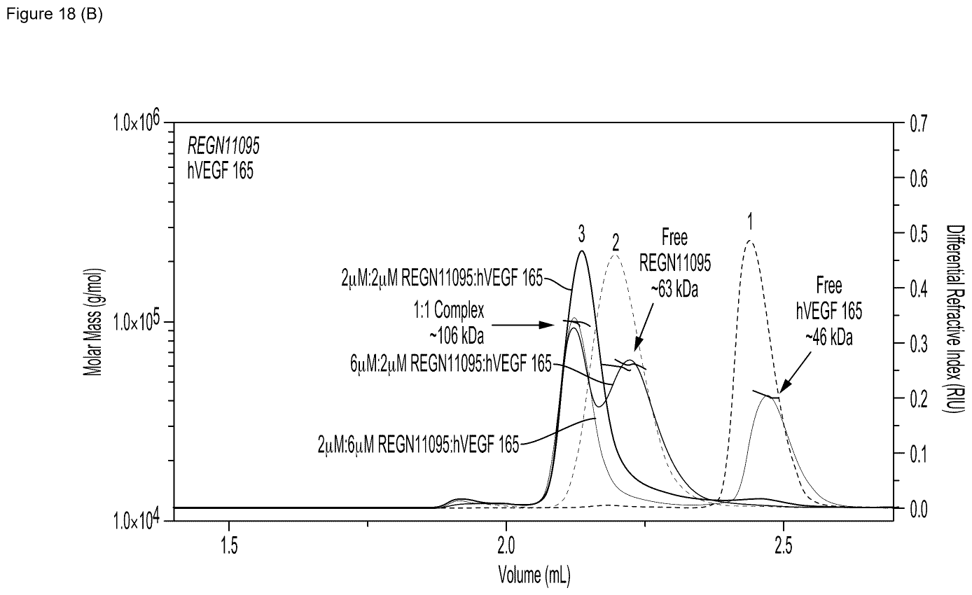

The stoichiometry of two individual mini-traps (REGN10105 and REGN11095) binding to human VEGF₁₆₅ (hVEGF₁₆₅; REGN110) was determined from SEC-MALS analysis of mini-trap in the presence and absence of varying concentrations of ligand. Under equivalent binding conditions, the stoichiometry of binding to hVEGF₁₆₅ Was equivalent for each mini-trap tested. The stoichiometries of the resulting complexes from all samples analyzed are presented in FIG. 18 (A), and FIG. 18 (B) and are summarized in Table 5-3 and Table 5-4.

Representative overlaid chromatograms corresponding to 3:1, 1:1, and 1:3 molar ratios of each mini-trap to hVEGF₁₆₅ are shown in FIGS. 18 (A) and 18 (B). The chromatograms of free hVEGF₁₆₅ and free mini-trap are also overlaid for comparison. The annotated peak numbers correspond to free hVEGF₁₆₅ (peak 1), free mini-trap (peak 2), and mini-trap-hVEGF₁₆₅ complexes (peak 3). The molar masses for each of the observed peaks are superimposed on the individual peaks for each chromatogram.

The measured molar masses of peak 1 and peak 2 were approximately 46 kDa and 63 kDa corresponding to free hVEGF₁₆₅ and free mini-trap, respectively. Based on the calculated molar masses of free hVEGF₁₆₅ and free mini-trap, the theoretical molar mass for a 1:1 complex of mini-trap:hVEGF₁₆₅ is predicted to be 109 kDa. Therefore, the chromatogram peak 3 most likely corresponds to a 1:1 mini-trap:hVEGF₁₆₅ complex based on a calculated average molar mass of approximately 106 kDa (Tables 5-3 and 5-4). Additionally, little to no higher molecular weight complexes were reliably detected for either sample under any of the tested conditions, indicating that each mini-trap binds to hVEGF₁₆₅ without higher-order multimerization. Taken together, the results demonstrate that each mini-trap exhibited equivalent binding stoichiometry to hVEGF₁₆₅, with each mini-trap molecule capable of binding one molecule of hVEGF₁₆₅ ligand.

TABLE 5-3
Summary of Molar Masses and Retention Tim of hVEGF₁₆₅ (REGN110)
Complexes with REGN10105

| | | | | | | Peak 3 | |
|---|---|---|---|---|---|---|---|
| | | | | | | [Mini-Trap]₁: | |
| | | Peak 1 | | Peak 2 | | [hVEGF₁₆₅]₁ | |
| | | Free hVEGF₁₆₅ | | Free Mini-Trap | | Complex | |
| | Molar Ratio | | | | | | M_{w}, |
| hVEGF₁₆₅ | (mol:mol) | Rₜ, min | M_{w}, kDa | Rₜ, min | M_{w}, kDa | Rₜ, min | kDa |
| hVEGF₁₆₅ | — | 2.4 | 45.7 | ND | ND | ND | ND |
| REGN10105 | — | ND | ND | 2.2 | 63.1 | ND | ND |
| REGN10105:hVEGF₁₆₅ | 3:1 | ND | ND | 2.2 | 62.7 | 2.1 | 104.1 |
| REGN10105:hVEGF₁₆₅ | 1:1 | ND | ND | ND | ND | 2.1 | 104.4 |
| REGN10105:hVEGF₁₆₅ | 1:3 | 2.5 | 45.5 | ND | ND | 2.1 | 106.2 |

Rₜ: Retention Time;
M_{w}: weight average molar mass;
NA: Not Applicable; min: minutes;
kDa: kiloDaltons;

TABLE 5-4
Summary of Molar Masses and Retention Time of hVEGF₁₆₅ (REGN110)
Complexes with REGN11095

| | | | | | | Peak 3 | |
|---|---|---|---|---|---|---|---|
| | | | | | | [Mini-Trap]₁: | |
| | | Peak 1 | | Peak 2 | | [hVEGF₁₆₅]₁ | |
| | | Free hVEGF₁₆₅ | | Free Mini-Trap | | Complex | |
| | Molar Ratio | | | | | | M_{w}, |
| Sample | (mol:mol) | Rₜ, min | M_{w}, kDa | Rₜ, min | M_{w}, kDa | Rₜ, min | kDa |
| hVEGF₁₆₅ | — | 2.4 | 45.7 | ND | ND | ND | ND |
| REGN11095 | — | ND | ND | 2.2 | 63.4 | ND | ND |
| REGN11095:hVEGF₁₆₅ | 3:1 | ND | ND | 2.2 | 65.8 | 2.1 | 106.4 |
| REGN11095:hVEGF₁₆₅ | 1:1 | ND | ND | ND | ND | 2.1 | 106.2 |
| REGN11095:hVEGF₁₆₅ | 1:3 | 2.5 | 45.8 | ND | ND | 2.1 | 106.6 |

Rₜ: Retention Time;
M_{w}: weight average molar mass;
NA: Not Applicable; min: minutes;
kDa: kiloDaltons;

### Example 6: Cryogenic Electron Microscopy (Cryo-EM) Structural Analysis of VEGF Mini-Trap REGN10105 in Complex with VEGF and Anti-VEGF Fab (REGN18)

Cryogenic electron microscopy was used to analyze the binding of REGN10105 to VEGF along with a Fab molecule. REGN18 is the anti-human VEGFR1-d2 mAb that is a non-blocker of VEGF binding which was added to increase the size of the complex to make the sample more suitable for Cryo-EM study.

Fab fragment preparation. The anti-hVEGFR1 domain 2 antibody REGN18 was cleaved into F(ab′)₂ and Fc fragments using Fabricator enzyme (Genovis) following standard protocols from the manufacturer. F(ab′)₂ was reduced into Fab using 2-mercaptoethylamine (2-MEA, ThermoFisher) followed by Fc fragment removal using CaptureSelect IgG-Fc (ms) affinity resin (ThermoFisher). Fab fragments were further purified by injection into a size exclusion chromatography (SEC) column (Superdex 200 Increase 15/300 GL, GE healthcare) connected to an AKTA Avant 25 chromatography system (GE healthcare). Running buffer contained 50 mM Tris-HCl pH 7.5, 150 mM NaCl. Peak fractions were pooled and concentrated in a 10 kDa cutoff centrifugal filter (Millipore Sigma) for subsequent use in complex preparation.

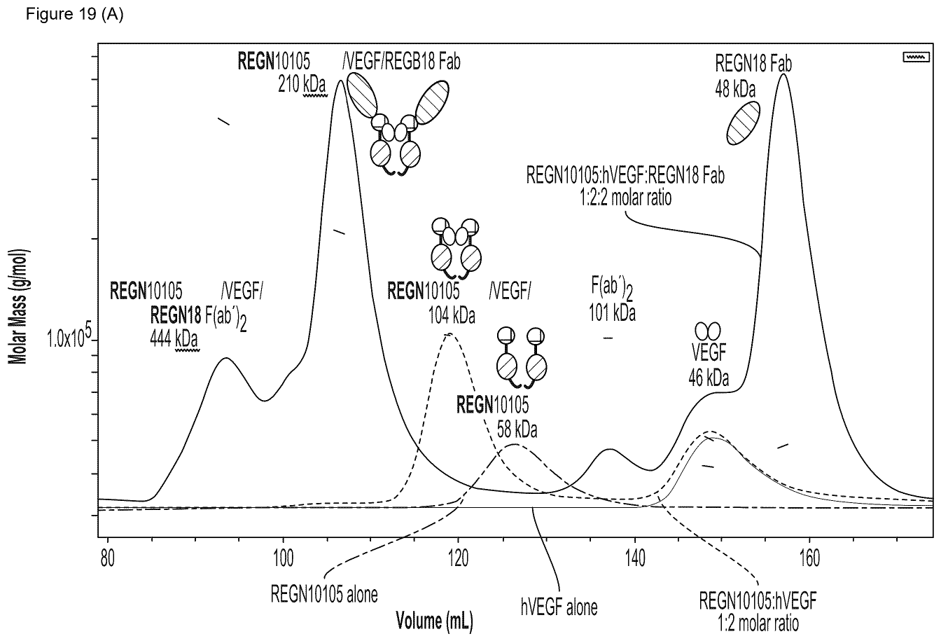

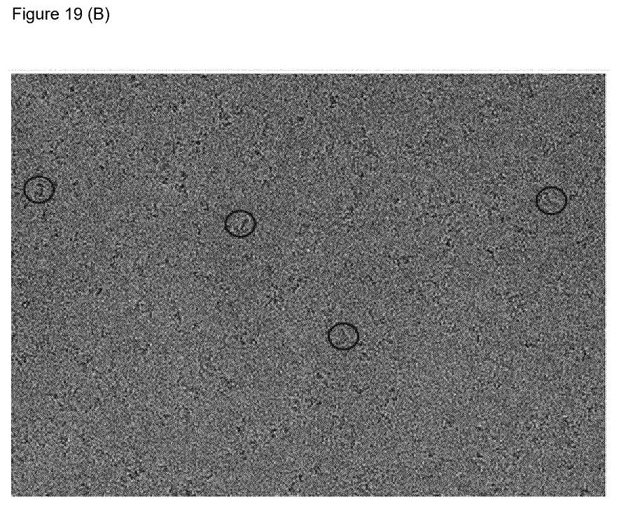

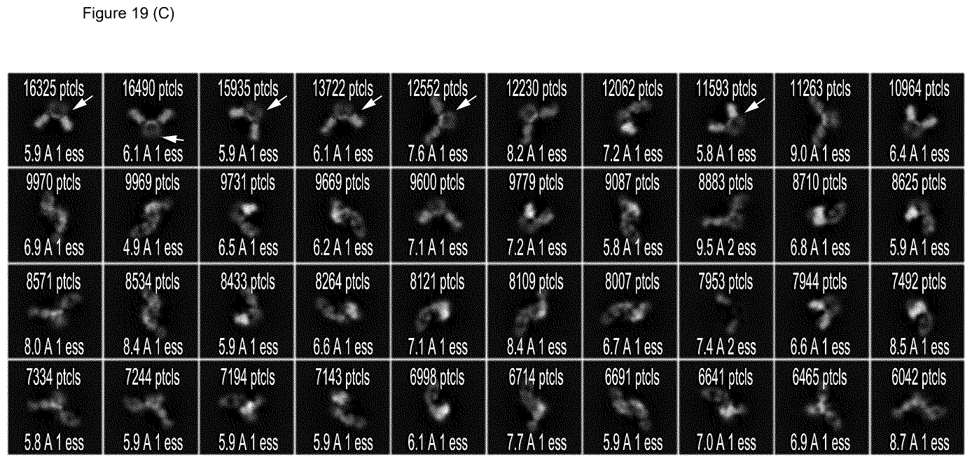

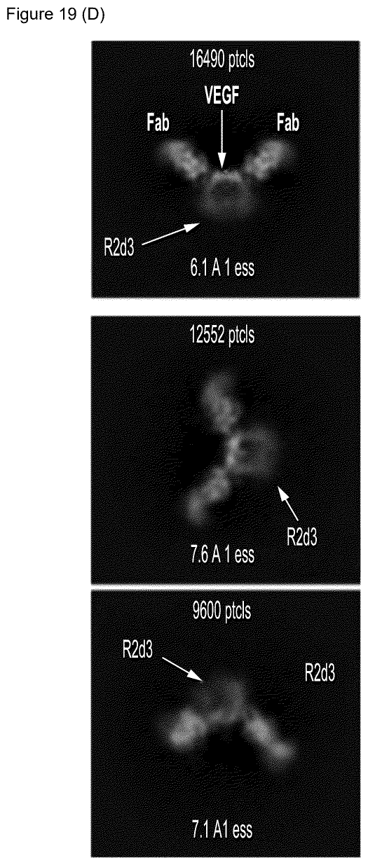

Complex preparation. 100 μg of purified mini-Trap (REGN10105-L3) was mixed with 200 μg REGN18 Fab and 160 μg hVEGF (REGN110-L9) and incubated at 4° C. overnight. The mixture was injected into an SEC column (Superdex 200 Increase 15/300 GL, GE) connected to an AKTA chromatography system (GE healthcare) and a Multi-Angle Light Scattering (MALS) detector (Wyatt Technology). SEC running buffer contained 50 mM Tris-HCl PH 7.5, 150 mM NaCl. Fractions from the peak corresponding to the REGN10105-REGN110-REGN18 Fab complex with an estimated molecular weight of 210 kDa were pooled and concentrated in a 30 kDa cutoff centrifugal filter (Millipore Sigma) to a concentration of 3.2 mg/mL measured using a Nanodrop instrument (ThermoFisher). See FIG. 19 (A). REGN18 is a non-blocker of VEGF binding and was added to increase the size of the REGN10105-REGN110 complex so as to make the sample more suitable for subsequent Cryo-EM study.

Cryo-EM sample preparation and data collection. Freshly purified REGN10105-REGN110-REGN18 Fab complex was mixed with 0.15% PMAL-C8 Amphipol detergent before pipetting 3.5 μL of the mixture onto a UltrAufoil R1.2/1.3, 300 mesh grid (Quantifoil). Excess liquid was blotted away using filter paper and the grid was plunge frozen into liquid ethane cooled by liquid nitrogen using a Vitrobot Mark IV (ThermoFisher) operated at 4° C. and 100% humidity. The grid was then loaded into a Titan Krios G3i microscope (ThermoFisher) equipped with a K3 camera (Gatan). 13,248 movies were collected in counted mode at a nominal magnification of 105,000× (0.85 Å pixel size). Each movie contained 46 dose fractions over a 2 second exposure, and the total acquired dose per Å2 was ˜40 electrons.

Cryo-EM data processing and map generation. Cryo-EM data were processed using Cryosparc v2.14.2. Movies were motion corrected by Patch motion correction and CTF parameters were estimated by Patch CTF estimation. Particles were initially picked using Blob picker to generate 2D class averages for template picking. 633,335 particles were left after running several rounds of 2D classification to remove junk particles. After ab initio reconstruction and Heterogenous refinement, 125,447 particles corresponding to the REGN10105-REGN110-REGN18 Fab complex were identified and further refined to 3.4 Å resolution reconstruction using Non-uniform refinement.

Results. Complex formation between REGN10105, hVEGF and REGN18 was determined. REGN10105 formed a 1:1 complex with hVEGF dimer (58 kDa+46 kDa=104 kDa). REGN10105, VEGF dimer and REGN18 Fab form a 1:1:2 complex (104 kDa+48 kDa*2=210 kDa). There was some REGN18 F(ab′)₂ contamination in the sample. See FIG. 19 (A).

The 2-dimensional structure of the REGN10105/VEGF/REGN18 complex was characterized by visualization under cryogenic electron microscopy. The 2D classes showed secondary structure features and some of them show clear 2-fold symmetry. The VEGFR2 domain 3 density is visible in many views, but it is quite blurry (arrow pointed) compared with the rest of the complex, suggesting that this domain is flexible. The VEGFR2 domain 3 density is even blurrier at the distal end.

A 3.4 Å Cryo-EM map of REGN10105-VEGF-REGN18 complex showed poor density at the VEGFR2-domain 3 C-terminal end. There was very clear density for VEGFR1 domain 2, VEGF and the REGN18 Fab variable domain; and poor density for the REGN18 Fab constant domain due to flexibility of this region. VEGFR2 domain 3 was resolved where the main chain and some side chains could be traced, but the Cryo-EM density is quite weak at the C-terminal end of R2-d3, likely due to high flexibility of this region. See FIG. 19 (B-D).

REGN10105 that forms a complex essentially as depicted in FIG. 19 (C-D) when complexed with human VEGF₁₆₅ and an anti-VEGF Fab, when visualized by Cryo-EM is part of the present invention.

### Example 7: Effect of Mini-Traps REGN10105 and REGN11095 in Oxygen Induced Retinopathy (OIR) Mouse Model

The effects of systemic and intravitreal REGN11095 and REGN10015 on retinal vasculature in an OIR mouse model was determined.

In the Oxygen Induced Retinopathy (OIR) model in mice, Taconic C57BI/6 mouse pups were placed in a hyperoxic environment (75% O₂) at postnatal day (P) 6 and returned to room air at P11. Study 1 (Intravitreal (IVT) Injection Equimolar Screen Study): OIR pups were injected IVT with human (h) Fc (hFc) 0.125 μg, aflibercept 0.25 μg, REGN10105 0.125 μg, REGN11095 0.125 μg respectively, at P13 and collected at P16. Study 2 (IVT Dose Response Study): OIR mice were injected IVT with hFc at 0.25 μg, REGN10105 at 0.025 μg, 0.25 μg, and 2.5 μg, or aflibercept at 0.05 μg, 0.5 μg, and 5 μg at P13 and collected at P16. Study 3 (Equimolar Systemic (IP) Study): OIR mice were injected IP with hFc at 5 mg/kg, aflibercept at 10 mg/kg, REGN10105 at 5 mg/kg, or REGN11095 at 5 mg/ml, respectively, at P12 and collected at P16. Retinas were fixed, dissected, and stained with FITC-labeled Griffornia simplicifolia lectin I (GS Lectin I) to label retinal vasculature (Vector Laboratories) and NG2 (Millipore) with secondary Alexa Fluor594 (Thermo Scientific) to label neovascular tufts.

Mice pups were placed in a chamber at the 75% O₂ at postnatal (P) 6 to P11. After 5 days in an oxygen chamber, they were placed back in room air (21% O₂). For Intravitreal (IVT) studies, mice were injected with reagents at P13 and eyes collected at P16. For systemic (IP) studies, mice were injected with reagents at P12 and eyes were collected at P16.

### Study 1: Intravitreal (IVT) Aflibercept and VEGF Mini-Trap Equimolar Dose Studies

Aflibercept (REGN3): 0.25 μg/eye
REGN11095: 0.125 μg/eye
REGN10105: 0.125 μg/eye
Vehicle (hFc): 0.125 μg/eye
   3 mice per group; left and right eyes injected

In the Oxygen Induced Retinopathy (OIR) model in mice, Taconic C57BI/6 mouse pups were placed in a hyperoxic environment (75% O₂) at postnatal day (P) 6 and returned to room air at P11.

Study 1 (IVT Equimolar Screen Study): OIR pups were injected IVT with human Fc (hFc) 0.125 μg, aflibercept 0.25 μg, REGN10105 0.125 μg, REGN11095 0.125 μg respectively, at P13 and collected at P16.

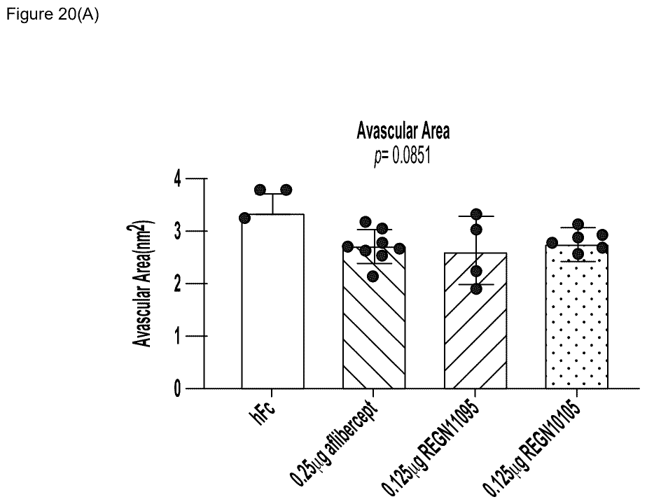

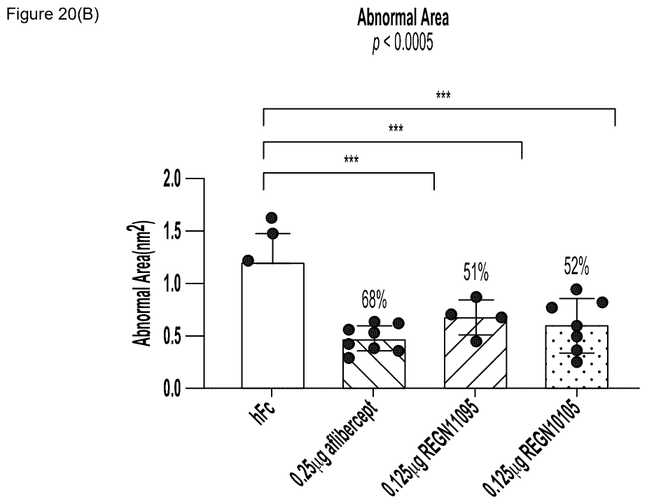

Retinas were fixed, dissected, and stained with FITC-labeled Griffornia simplicifolia lectin I (GS Lectin I) to label retinal vasculature (Vector Laboratories) and NG2 (Millipore) with secondary Alexa Fluor594 (Thermo Scientific) to label neovascular tufts. Avascular area of mouse retinas are summarized in FIG. 20 (A). The abnormal area of the mouse retinas are summarized in FIG. 20 (B).

Study 2: Intravitreal (IVT) Equimolar Aflibercept and VEGF mini-Trap Dose Response studies
Aflibercept (REGN3): 0.05 μg/eye, 0.5 μg/eye, and 5 μg/eye
REGN10105: 0.025μ g/eye, 0.25μ g/eye, and 2.5μ g/eye
Vehicle (hFc): 0.25 μg/eye
   3-4 mice per group; only left eyes injected

In the Oxygen Induced Retinopathy (OIR) model in mice, Taconic C57BI/6 mouse pups were placed in a hyperoxic environment (75% O₂) at postnatal day (P) 6 and returned to room air at P11.

Study 2 (IVT Dose Response Study): OIR mice were injected IVT with hFc at 0.25 μg, REGN10105 at 0.025 μg, 0.25 μg, and 2.5 μg, or aflibercept at 0.05 μg, 0.5 μg, and 5 μg at P13 and collected at P16.

Retinas were fixed, dissected, and stained with FITC-labeled Griffornia simplicifolia lectin I (GS Lectin I) to label retinal vasculature (Vector Laboratories) and NG2 (Millipore) with secondary Alexa Fluor594 (Thermo Scientific) to label neovascular tufts.

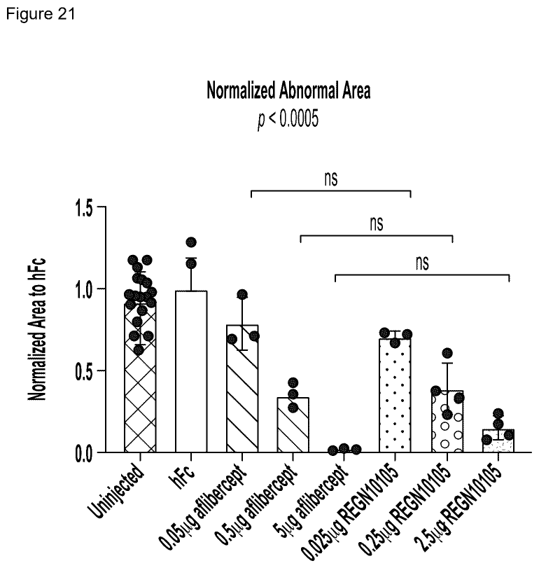

The area of abnormal retinal area, which is normalized to that of mice dosed with hFc only, is set forth in FIG. 21.

### Study 3: Systemic Aflibercept and VEGF Mini-Trap Equimolar Studies

Aflibercept (REGN3): 10 mg/kg
REGN10105 5 mg/kg
REGN11095 5 mg/kg
Vehicle (hFc): 5 mg/kg
   *Experiment done 2 times
   3 mice per group; left and right eyes analyzed

In the Oxygen Induced Retinopathy (OIR) model in mice, Taconic C57BI/6 mouse pups were placed in a hyperoxic environment (75% O₂) at postnatal day (P)6 and returned to room air at P11.

Study 3 (Equimolar Systemic (IP) Study): OIR mice were injected IP with hFc at 5 mg/kg, aflibercept at 10 mg/kg, REGN10105 at 5 mg/kg, or REGN11095 at 5 mg/ml, respectively, at P12 and collected at P16.

Retinas were fixed, dissected, and stained with FITC-labeled Griffornia simplicifolia lectin I (GS Lectin I) to label retinal vasculature (Vector Laboratories) and NG2 (Millipore) with secondary Alexa Fluor594 (Thermo Scientific) to label neovascular tufts.

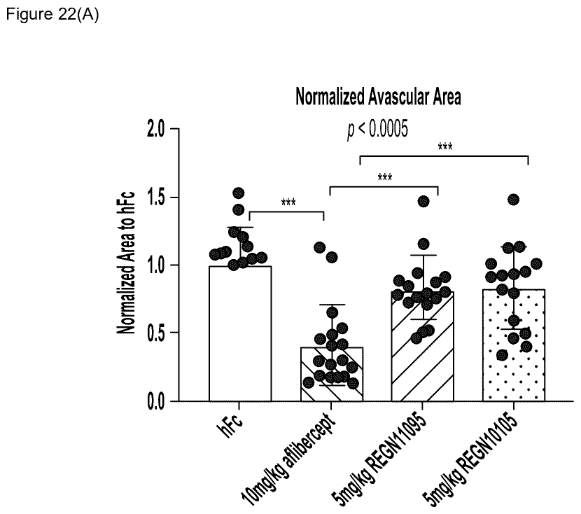

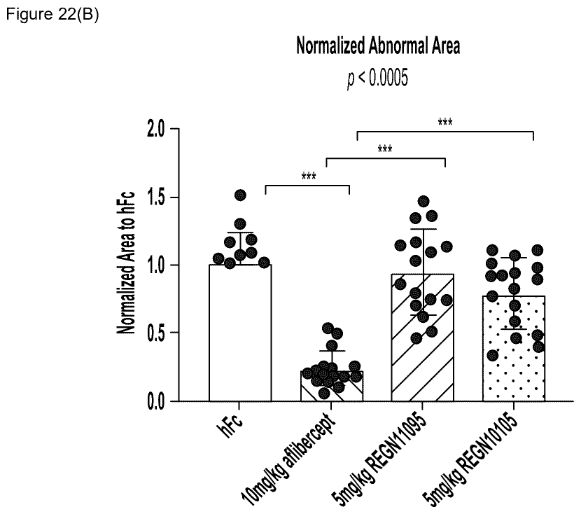

The avascular area of mouse retinas, normalized to that of mice dosed with hFc only, are summarized in FIG. 22 (A). The abnormal area of the mouse retinas, normalized to that of mice dosed with hFc only, are summarized in FIG. 22 (B).

### Example 8: VEGF Trap and Mini-Trap (REGN10105, REGN11095 and REGN3) In Vivo Pharmacokinetics (PK) in Rabbits

The pharmacokinetics of REGN10105 and REGN11095 in rabbits was investigated.

This PK study showed that the half-lives of the VEGF mini-traps (REGN10105 and REGN11095)) were shorter than that of VEGF Trap (REGN3) in NZW rabbit vitreous, the VEGF mini-traps persistence was about 22% and 32% shorter, respectively (5.0 days vs 3.9 and 3.4 days).

TABLE 8-1
VEGF Traps and VEGF Mini-Traps Tested

| | | IgG hinge amino |
|---|---|---|
| REGN# | Common name | acid sequence |
| REGN3 | VEGF Trap | DKTHT CPPCP |
| | hIgG1 Fc | APELLGGPSV |
| | | (SEQ ID NO: 107) |
| REGN10105 | VEGF mini-trap | VE CPPCP APPVA |
| | hIgG2 Fc w_2 | (SEQ ID NO: 5) |
| | cys | |
| REGN11095 | VEGF mini-trap | DKTHT CPPCP GGGLLG |
| | hIgG1 Mut | (SEQ ID NO: 13) |
| | GGGL (SEQ ID | |
| | NO: 94) | |

Experimental procedures. VEGF Trap (REGN3) and two new VEGF mini-traps (REGN10105, and REGN11095) were molecules tagged with Alexa Fluor 488 (AF488) through amine conjugation. Protein concentrations, endotoxin levels, and Degree of Labeling (DOL) are provided in Table 8-2. Bilateral intravitreal (IVT) injections were made to 6 male New Zealand White (NZW) rabbits (6 eyes/3 rabbits/molecule). All eyes were examined for vitreous baseline fluorescence with OcuMetrics Fluorotron fluorophotometer (Mountain View, CA) before injection, and followed up for vitreous fluorescence intensity post injection at Day 3, 7, 14 and 21. General ocular examination included intraocular pressure (IOP), inflammation signs, corneal and conjunctival edema, hemorrhages, floaters in anterior chamber, pupil size and shape, cataract, and retinal detachment before and 30 minutes after IVT injection, and at each follow-up time point. Fluorescence intensity and position information were extracted and imported in GraphPad Prism for graphical display and analysis. The data were fitted to a first order, single compartment model.

TABLE 8-2
Half-life (t_{1/2}) of VEGF Trap and VEGF Mini-Trap in Intravitreal
Photofluorimetry PK in Rabbits

| | | | | DOL | | |
|---|---|---|---|---|---|---|
| | | | | (Degree | | |
| | | | Endotoxin | of AF488 | Volume | t_{1/2} (Std) |
| Test article | Conjugation | Conc. mg/ml | (EU/mL) | Labeling) | (uL)/eye | Days |
| REGN3 | AF488 | 2.29 | <5 | 2.38 | 50 | 5.0 (0.4) |
| (aflibercept) | | | | | | |
| REGN10105 | AF488 | 2.92 | <1.05 | 2.87 | 50 | 3.9 (0.3) |
| REGN11095 | AF488 | 2.93 | <1.05 | 2.65 | 50 | 3.4 (0.5) |

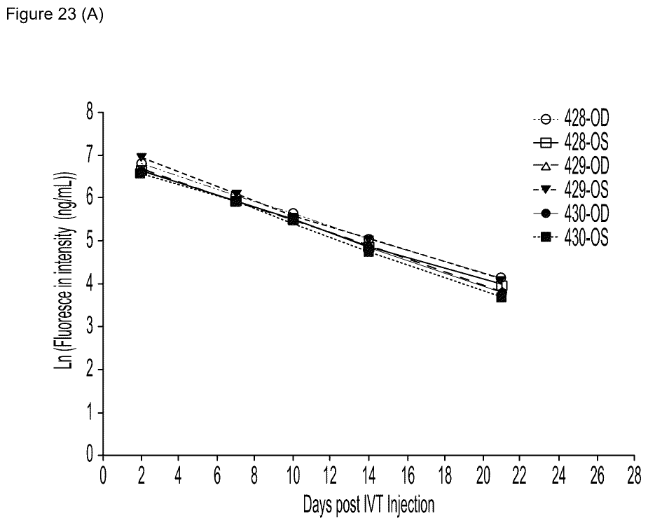

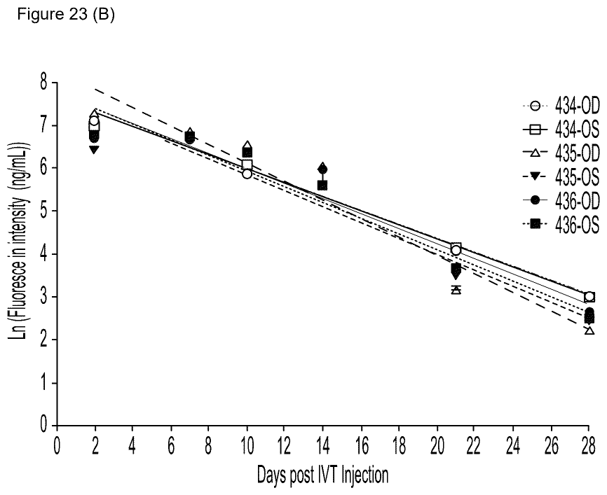

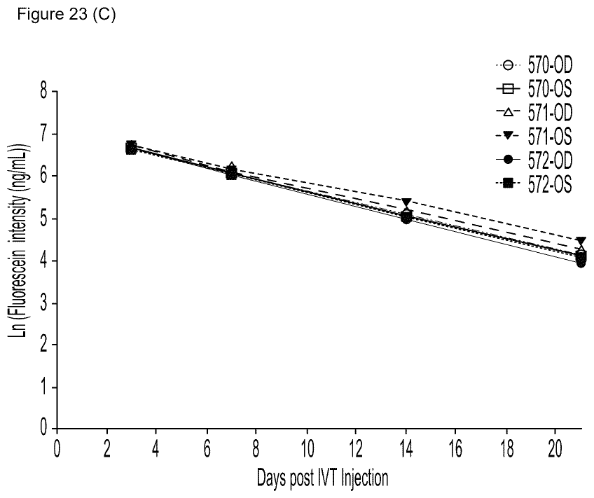

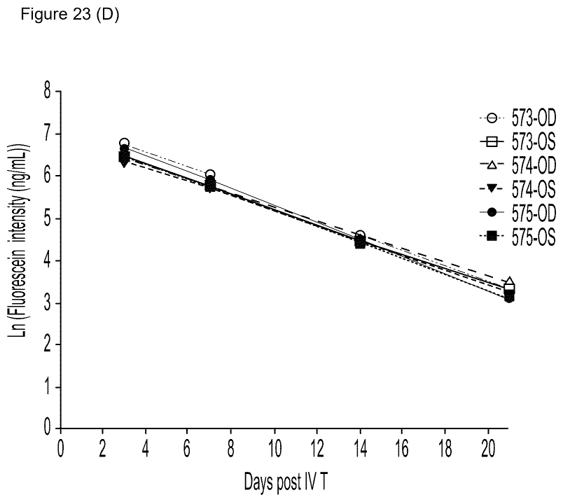

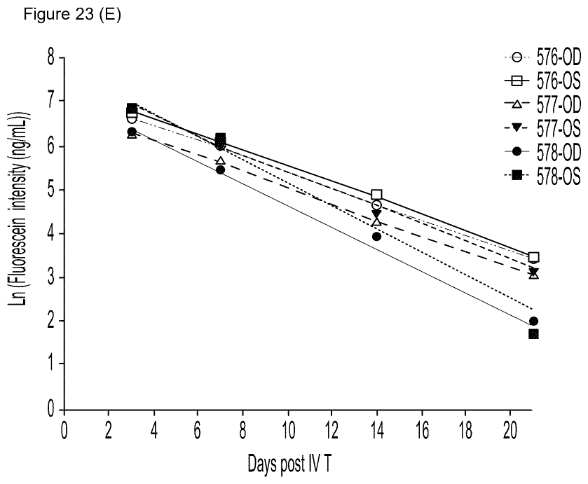

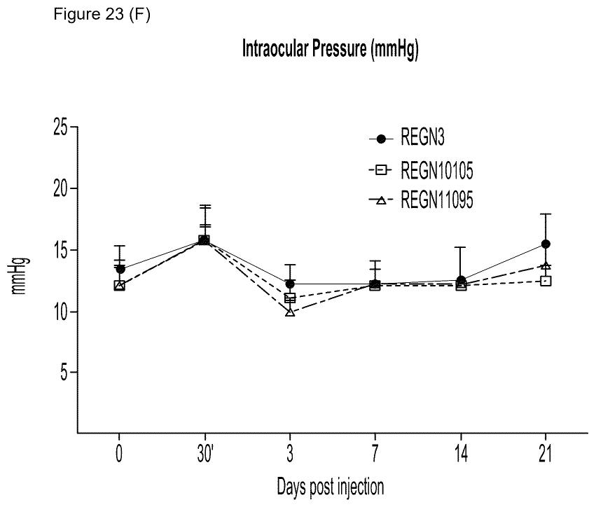

Results. The PK study of VEGF Trap (REGN3) and the VEGF mini-traps (REGN10105 and REGN11095) in NZW rabbit vitreous showed the half-lives were 5.0 (±0.4), 3.9 (±0.3), and 3.4 (±0.5) days, respectively. See FIG. 23 (A-C) There was no significant IOP change before and after IVT injection of either molecule. See FIG. 23 (D). There were no clinically notable signs observed in general ocular examination.

### Example 9: Pharmacokinetic Study of VEGF Mini-Traps in Cynomolgus Monkeys

The pharmacokinetics of REGN10105 and REGN11095 in monkeys was investigated.

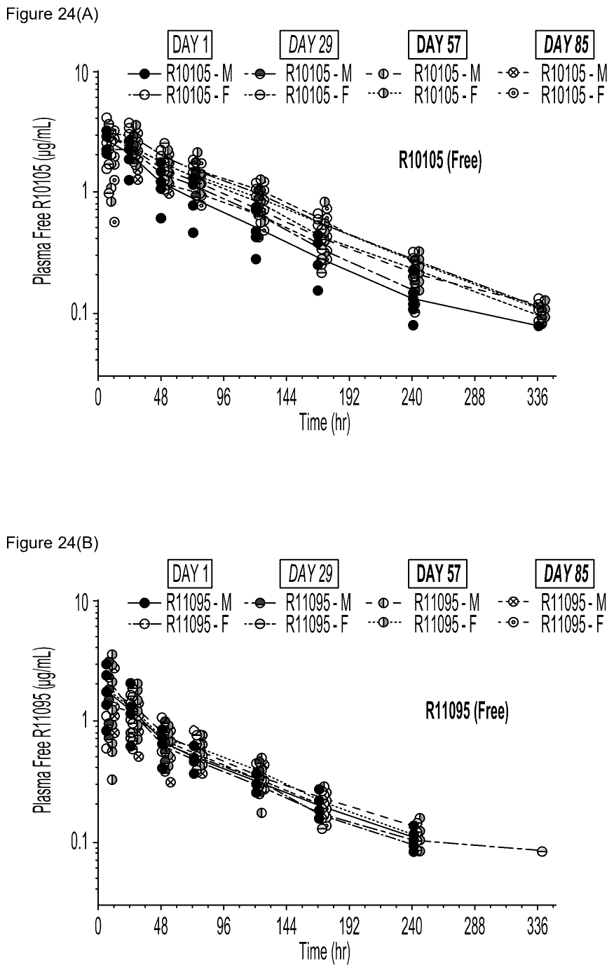

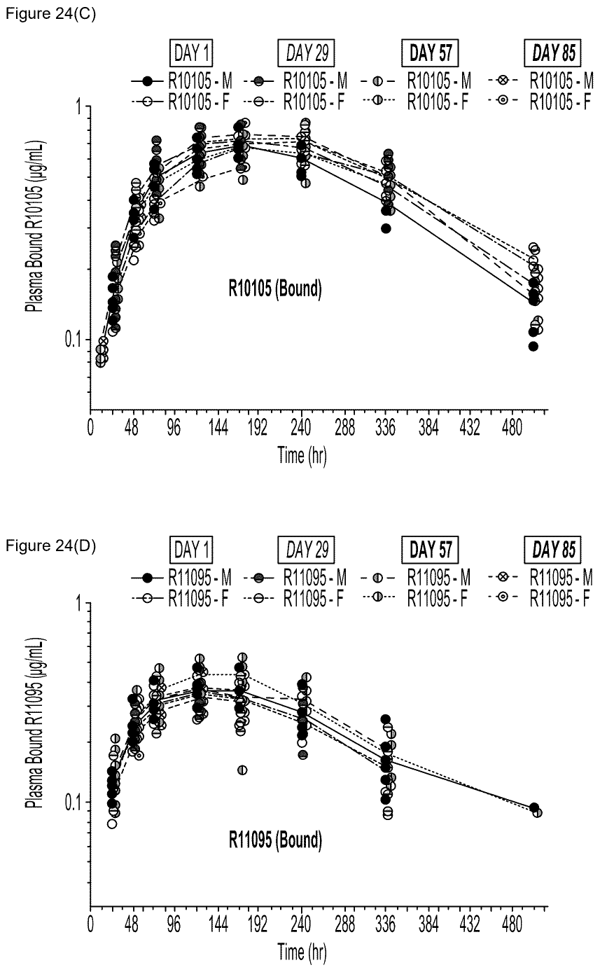

Concentrations of free and bound REGN10105 and REGN11095 were measured in plasma of cynomolgus monkeys (6/sex/group) following intravitreal administration. In this study, anesthetized animals received bilateral intravitreal injections of REGN10105 or REGN11095 every 4 weeks for a total of 4 doses at 5.5 mg/eye/dose. A1-cc syringe and 30-gauge needle was used for each dose administration of 50 microL/eye. Blood samples were collected after each of the 4 doses for systemic drug exposure evaluation. The assays used to measure plasma concentrations of free and bound REGN10105 and REGN11095 were enzyme-linked immunosorbent assay-based methods with a lower limit of quantitation of 0.078 μg/mL for free and bound species of each compound. See FIG. 24 (A-D).

Peak plasma concentrations of free REGN10105 were achieved by the first sampling time (6 hours) while plasma concentrations of bound REGN10105 were delayed and generally between 5 to 7 days after dosing. Plasma concentrations of free or bound REGN10105 were similar in male and female monkeys after the first dose as well as after each of the subsequent (total of 4 doses) doses of REGN10105 indicating that there were no gender differences and no systemic drug accumulation after multiple dosing. Animals dosed with REGN11095, while they shared similar plasma pharmacokinetic characteristics as REGN10105 (e.g., time to peak concentrations, lack of gender differences and no accumulation after multiple dosing), showed lower plasma concentrations and exposure than animals dosed with REGN10105.

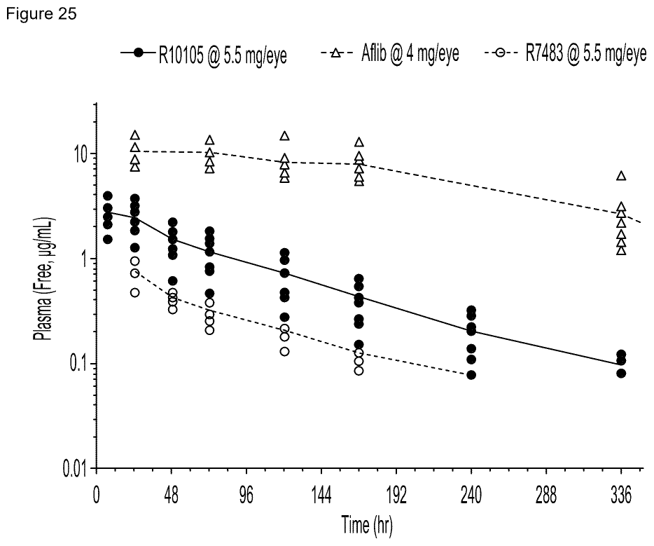

A comparison of free REGN10105 plasma concentrations, after a single dose (the first dose of the multiple dose study), with plasma concentration profiles from REGN7483 and REGN3 shows that REGN7483 has the lower plasma concentrations (systemic exposure), followed by REGN10105, than that of REGN3 when given as intravitreal injections at similar dosages (5.5 mg/eye of either REGN10105 or REGN7483 or 4 mg/eye of REGN3). This indicates that both REGN7483 and REGN10105 have lower systemic exposure after intravitreal injections to each eye than REGN3. See FIG. 25.

### Example 10: N-Linked Glycan Analysis of VEGF Mini-Trap

The glycan profile of REGN10103 and REGN10105, in particular the levels of fucosylation, galactosylation, sialylation, high mannose and bisecting glycation, was determined in this example.

N-linked glycans were released from the protein by PNGase-F in 50 mM HEPES buffer (pH 7.9) containing 0.1% Waters RapiGest and 4.2 mM TCEP at 45° C. for 25 minutes. The released glycans were labeled with RapiFluor-MS fluorescence dye at 45° C. for 30 minutes. The protein was precipitated by adding 25% DMF and 53% [v/v] acetonitrile and pelletized to the bottom of the tube through centrifugation at 16,000×g for 5 minutes. The supernatant containing the labeled glycans was collected and analyzed on an UPLC using hydrophilic interaction liquid chromatography (Waters BEH Amide column) with post-column fluorescence detection. After binding to the column, the labeled glycans were separated using a binary mobile phase gradient comprised of aqueous 50 mM ammonium formate (pH 4.4) as mobile phase A and acetonitrile as mobile phase B. The separated glycans were detected using a fluorescence detector with an excitation wavelength of 265 nm and an emission wavelength of 425 nm. Using the relative area percentages of the N-glycan peaks in the resultant chromatograms, the N-glycan distribution was reported as the total percentage of N-glycans (1) containing a core fucose residue, (2) containing at least one sialic acid residue, (3) identified as Mannose-5, (4) containing at least one galactose residue, and (5) bisecting N-glycans.

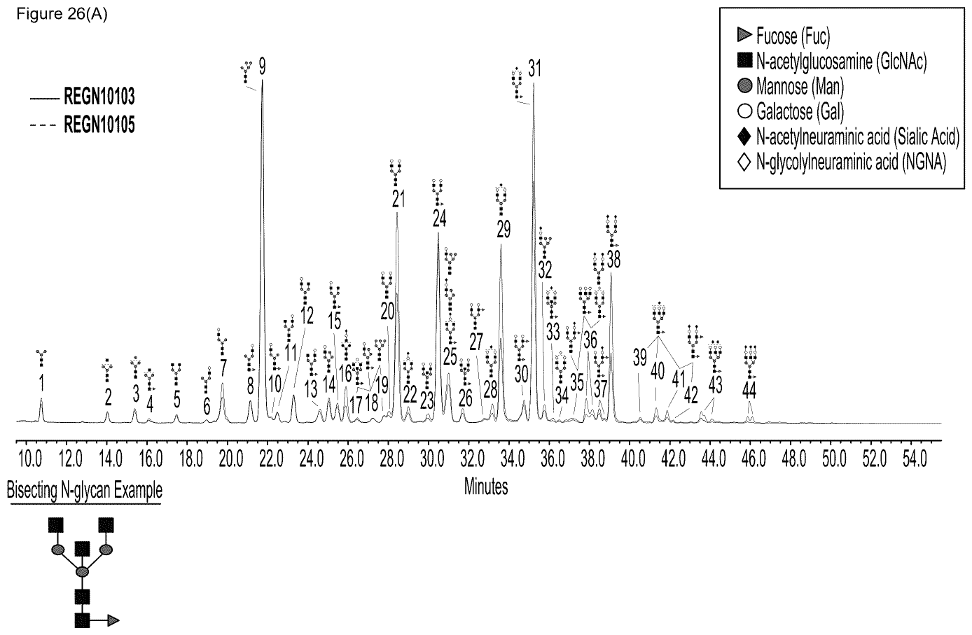

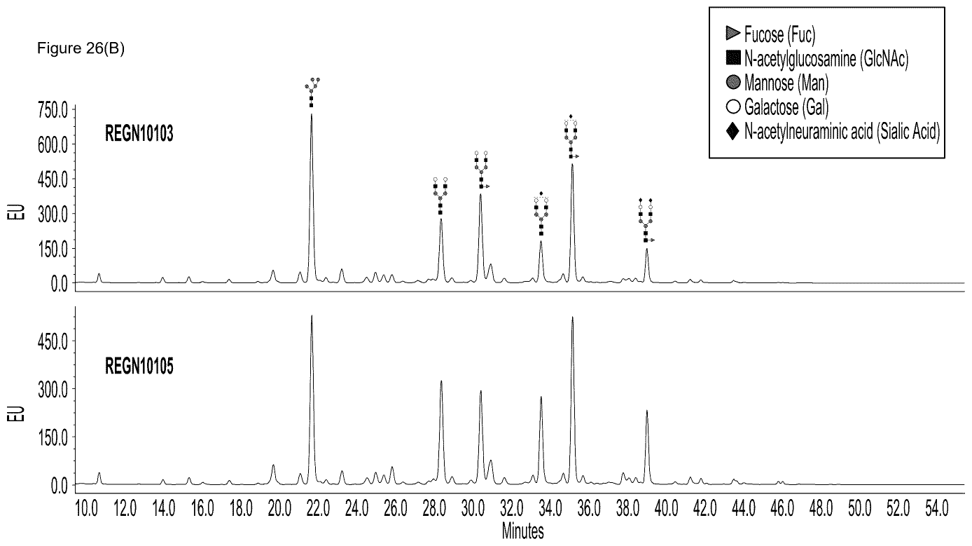

The overlaid HILIC-FLR (hydrophilic interaction chromatography-fluorescence) chromatograms of N-linked glycans from REGN10103 and REGN10105 are set forth in FIG. 26(A) and the separate chromatograms are set forth in FIG. 26(B) and summarized in Table 10-1 herein.

TABLE 10-1
Summary of Glycan Quantification

| Glycosylation | REGN10103 | REGN10105 |
|---|---|---|
| % Fucosylation | 43.3% | 44.8% |
| % Galactosylation | 64.4% | 71.6% |
| % Sialylation | 20.0% | 26.5% |
| % High Mannose | 25.2% | 18.6% |
| % Bisecting | 1.6% | 1.9% |

There was an elevated level of high-mannose species (for example, Man5) in REGN10103 relative to REGN10105. The relative abundance of galactosylation and sialylation were lower in REGN10103 than in REGN10105 possibly due to the elevated high-mannose in REGN10103.

### Example 11: REGN10105 Viscosity and Stability Study

Stability (stress stability and photostability) and viscosity studies of REGN10105 was performed in this example.

REGN10105 (IgG2, 2 Cys, FabRICATOR cleaved VEGF mini-trap) was developed and found to eliminate the risk of anti-hinge antibodies associated with Fc-cleaved IgG1 molecule, REGN7483. The viscosity, thermal stability, and photostability of REGN10105 was evaluated in a composition including histidine.

The viscosity assessment was performed for REGN10105 at concentrations ranging between 38-132 mg/mL. A concentration versus viscosity graph was plotted and the results were compared with REGN7483. REGN10105 showed lower viscosity profile as compared to REGN7483 at equimolar concentrations (viscosity=5.8 cP for REGN10105 (at 90 mg/ml) versus 6.9 cP for REGN7483 (at 90 mg/ml)). The viscosity of about 12 cP was observed at concentration of 120 mg/mL REGN10105. The viscosity increased exponentially at concentrations above 120 mg/mL.

Thermal stability of REGN10105 was evaluated at 90 mg/ml and 120 mg/mL in Type I, borosilicate glass vials at a fill volume of 0.4 mL. Thermal stress stability was tested at 37° C. for 4 weeks and storage stability was assessed at 5° C. up to 3 months. The data generated was compared with the results from the stability testing for 90 mg/mL REGN7483F. REGN10105 showed lower aggregation rates under thermal stress (3.74% HMW/week) as compared to REGN7483 (5.45% HMW/week) at 90 mg/mL. The storage stability for REGN10105 was comparable to REGN7483 at similar concentrations. In addition, REGN10105 formulation did not show any obvious change in aggregation levels on agitation (1000 rpm up to 15 min) and freeze thaw (2×).

The photostability of 10 mg/mL REGN10105 was evaluated in the photochamber (Bahnson ES2000 CL-LT Photo-Stability Chamber) after exposure to ambient (10 W/m² for 0.6 h and cool-white light (CWL) at 8Klux for 18 h (6 W*h/m² and 144 klux*h)) and 0.5×ICH (10W/m² for 10 h and 8Klux for 75h (100W*h/m² and 600Klux*h)). REGN10105 showed slightly better photostability than REGN7483. Overall, REGN10105 showed higher thermal stability and lower viscosity as compared to REGN7483.

All references cited herein are incorporated by reference to the same extent as if each individual publication, database entry (e.g., Genbank sequences or GeneID entries), patent application, or patent, was specifically and individually indicated to be incorporated by reference. This statement of incorporation by reference is intended by Applicants to relate to each and every individual publication, database entry (e.g., Genbank sequences or GeneID entries), patent application, or patent, each of which is clearly identified in even if such citation is not immediately adjacent to a dedicated statement of incorporation by reference. The inclusion of dedicated statements of incorporation by reference, if any, within the specification does not in any way weaken this general statement of incorporation by reference. Citation of the references herein is not intended as an admission that the reference is pertinent prior art, nor does it constitute any admission as to the contents or date of these publications or documents.

We claim:

1. A VEGF mini-trap comprising a polypeptide characterized by the domain structure: 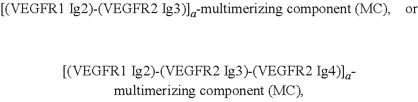
wherein a=1, 2, 3, 4, 5, 6, 7, 8, 9 or 10; and
wherein the MC comprises, consists of or consists essentially of a hinge region which is
an IgG Fc domain comprising the upper hinge region, the core hinge region and the lower hinge region which extends up to, but does not include the GPSV (SEQ ID NO: 96) cleavage site for IdeS protease, or a variant thereof with the proviso that the hinge region does not consist of the amino acid sequence DKTHTCPPCPAPELLG (amino acids 6-21 of SEQ ID NO: 15), DKTHTCPPCPPC (SEQ ID NO: 87); DKTHTCPPCPPCPPC (SEQ ID NO: 88) or DKTHTCPLCPAPELLG (SEQ ID NO: 89);
an IgG1 hinge region wherein residues ELLG (SEQ ID NO: 90) are mutated to PVA;
an IgG1 hinge region-IgG2 hinge region chimera wherein the IgG1 residues ELLG (SEQ ID NO: 90) are mutated to PVA;
an IgG1 hinge region wherein residues ELLG (SEQ ID NO: 90) are mutated to GGG;
an IgG1 hinge region wherein residues APEL (SEQ ID NO: 91) mutated to GGGF (SEQ ID NO: 92), GGGG (SEQ ID NO: 93) or GGGL (SEQ ID NO: 94);
an IgG1 hinge wherein APE residues are deleted;
an IgG2 hinge region;
an IgG3 hinge region;
an IgG4 hinge region;
an IgG4 hinge region wherein IgG4 hinge residues EFLG (SEQ ID NO: 95) are mutated to PVA; or
an IgG4 hinge region wherein residues EFLG (SEQ ID NO: 95) are mutated to GGG.

2. A VEGF trap comprising a polypeptide characterized by the domain structure: 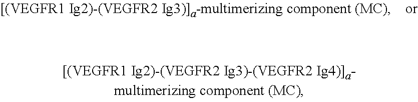
wherein a=1, 2, 3, 4, 5, 6, 7, 8, 9 or 10; and
wherein the MC comprises a human Fc hinge region which is
an IgG1 hinge region comprising a stealth mutation;
an IgG1 hinge region-IgG2 hinge region chimera;
an IgG1 hinge region with one or more of residues APELLG (SEQ ID NO: 108) or PEL mutated;
an IgG1 with APE residue deletion;
an IgG2 hinge region;
an IgG3 hinge region;
an IgG4 hinge region; or
an IgG4 hinge region comprising a stealth mutation;
wherein the MC comprises a protease cleavage site.

3. A VEGF mini-trap that comprises, consists of or consists essentially of the amino acid sequence:
(SEQ ID NO: 32, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKERKCCVECPPCPAPPVA
.
,
(SEQ ID NO: 34, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPPVA
(SEQ ID NO: 35, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKESKYGPPCPPCPAPPVA
(SEQ ID NO: 36, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPPVA
(SEQ ID NO: 37, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPGGGFLG
(SEQ ID NO: 38, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPGGGGLG
(SEQ ID NO: 39, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPGGGLLG
(SEQ ID NO: 40, or a variant thereof).
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPLLG

4. A VEGF Trap that comprises, consists of or consists essentially of an amino acid sequence:
(SEQ ID NO: 41, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKVECPPCPAPPVAGPSVFLFPPKPKDTLMISRTPEVTCVVVDVSHE
DPEVQFNWYVDGVEVHNAKTKPREEQFNSTFRVVSVLTVVHQDWLNGKEY
KCKVSNKGLPAPIEKTISKTKGQPREPQVYTLPPSREEMTKNQVSLTCLV
KGFYPSDIAVEWESNGQPENNYKTTPPMLDSDGSFFLYSKLTVDKSRWQQ
GNVFSCSVMHEALHNHYTQKSLSLSPGK
(SEQ ID NO: 42, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPPVAGPSVFLFPPKPKDTLMISRTPEVTCVVVDV
SQEDPEVQFNWYVDGVEVHNAKTKPREEQFNSTYRVVSVLTVLHQDWLNG
KEYKCKVSNKGLPSSIEKTISKAKGQPREPQVYTLPPSRDELTKNQVSLT
CLVKGFYPSDIAVEWESNGQPENNYKTTPPVLDSDGSFFLYSKLTVDKSR
WQQGNVFSCSVMHEALHNHYTQKSLSLSPGK
(SEQ ID NO: 43, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKERKCCVECPPCPAPPVAGPSVFLFPPKPKDTLMISRTPEVTCVVV
DVSHEDPEVQFNWYVDGVEVHNAKTKPREEQFNSTFRVVSVLTVVHQDWL
NGKEYKCKVSNKGLPAPIEKTISKTKGQPREPQVYTLPPSREEMTKNQVS
LTCLVKGFYPSDIAVEWESNGQPENNYKTTPPMLDSDGSFFLYSKLTVDK
SRWQQGNVFSCSVMHEALHNHYTQKSLSLSPGK
(SEQ ID NO: 44, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKESKYGPPCPPCPAPPVAGPSVFLFPPKPKDTLMISRTPEVTCVVV
DVSQEDPEVQFNWYVDGVEVHNAKTKPREEQFNSTYRVVSVLTVLHQDWL
NGKEYKCKVSNKGLPSSIEKTISKAKGQPREPQVYTLPPSQEEMTKNQVS
LTCLVKGFYPSDIAVEWESNGQPENNYKTTPPVLDSDGSFFLYSRLTVDK
SRWQEGNVFSCSVMHEALHNHYTQKSLSLSLGK
(SEQ ID NO: 45, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPAPPVAGPSVFLFPPKPKDTLMISRTPEVTCVVVDV
SHEDPEVQFNWYVDGVEVHNAKTKPREEQFNSTFRVVSVLTVVHQDWLNG
KEYKCKVSNKGLPAPIEKTISKTKGQPREPQVYTLPPSREEMTKNQVSLT
CLVKGFYPSDIAVEWESNGQPENNYKTTPPMLDSDGSFFLYSKLTVDKSR
WQQGNVFSCSVMHEALHNHYTQKSLSLSPGK
(SEQ ID NO: 46, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPGGGFLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK
(SEQ ID NO: 47, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPGGGGLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK
(SEQ ID NO: 48, or a variant thereof);
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPGGGLLGGPSVFLFPPKPKDTLMISRTPEVTCVVVD
VSHEDPEVKFNWYVDGVEVHNAKTKPREEQYNSTYRVVSVLTVLHQDWLN
GKEYKCKVSNKALPAPIEKTISKAKGQPREPQVYTLPPSRDELTKNQVSL
TCLVKGFYPSDIAVEWESNGQPENNYKTTPPVLDSDGSFFLYSKLTVDKS
RWQQGNVFSCSVMHEALHNHYTQKSLSLSPGK
or
(SEQ ID NO: 49, or a variant thereof).
SDTGRPFVEMYSEIPEIIHMTEGRELVIPCRVTSPNITVTLKKFPLDTLI
PDGKRIIWDSRKGFIISNATYKEIGLLTCEATVNGHLYKTNYLTHRQTNT
IIDVVLSPSHGIELSVGEKLVLNCTARTELNVGIDFNWEYPSSKHQHKKL
VNRDLKTQSGSEMKKFLSTLTIDGVTRSDQGLYTCAASSGLMTKKNSTFV
RVHEKDKTHTCPPCPLLGGPSVFLFPPKPKDTLMISRTPEVTCVVVDVSH
EDPEVKFNWYVDGVEVHNAKTKPREEQYNSTYRVVSVLTVLHQDWLNGKE
YKCKVSNKALPAPIEKTISKAKGQPREPQVYTLPPSRDELTKNQVSLTCL
VKGFYPSDIAVEWESNGQPENNYKTTPPVLDSDGSFFLYSKLTVDKSRWQ
QGNVFSCSVMHEALHNHYTQKSLSLSPGK

5. The VEGF mini-trap of claim 1 which is glycosylated.

6. The VEGF mini-trap of claim 1 wherein
said VEGF mini-trap comprises N-linked glycans;
said VEGF mini-trap comprises O-linked glycans;
said VEGF mini-trap is sialylated, galactosylated and/or fusosylated at one or more residues;
said VEGF mini-trap comprises an N-linked mannose-5 glycan (Man5) and/or a bisecting N-glycan at one or more residues; and/or
said VEGF mini-trap comprises CHO cell glycosylation.

7. The VEGF mini-trap of claim 1, characterized by any one or more of the following:
Binds to human VEGF₁₆₅ with a K_{D} of about 1-2 pM or a greater affinity at 25° C. wherein said affinity was measured by surface plasmon resonance;
Has a molecular weight of less than 97 kDa;
Has an IC₅₀ of about 1-2×10⁻¹¹ M for blocking VEGF₁₂₁ and/or VEGF₁₆₅ activation of VEGFR1 and/or VEGFR2 as measured in HEK293 cells expressing the VEGF receptor extracellular domain fused to the intracellular domain of IL18Ralpha or IL18R beta and having an NFkappaB-luciferase-IRES-eGFP reporter gene;
Exhibits less immunogenicity in monkey naïve serum samples and/or human AMD/DME baseline serum samples than that of REGN7483 as measured by anti-drug antibody (ADA) assay;
Forms a 1:1 complex with VEGF₁₆₅ homodimer;
Forms a 1:1:2 complex with VEGF₁₆₅ homodimer and an anti-VEGF Fab fragment (VEGF mini-trap:VEGF₁₆₅ homodimer:anti-VEGF Fab fragment);
Reduces retinal neovascularization when dosed intravitreally in an Oxygen Induced Retinopathy mouse model;
Exhibits a shorter intravitreal half-live than aflibercept in rabbit eyes when intravitreally injected;
Has a half-life of about 3.4 or 3.9 days in the vitreous of a rabbit when injected with about 0.146 mg VEGF mini-trap;
Exhibits lower plasma levels of free molecule when injected intravitreally at about 5.5 mg/eye, than aflibercept injected intravitreally at 4 mg/eye in a non-human primate;
Exhibits about 0.1 or 0.2 or 0.1-0.2 micrograms/ml (or less) concentration of free molecule in the plasma of a non-human primate about 336 hours after intravitreal injection of about 5.5 mg/eye;
Does not significantly accumulate in the eye of a non-human primate after four intravitreal injections about every 28 days of about 5.5 mg/eye;
Exhibits less formation of high molecular weight species than aflibercept and/or REGN7483 when stored at 37° C. for 4 weeks;
Has a viscosity of about 5.8 cP at 90 mg/ml concentration and/or about 12.1 cP at 120 mg/ml concentration at 20° C.;
Forms HMW species when exposed to light at a rate of about 8.8% increase in HMW/M lux*hrs (relative to a dark control);
one or more histidines are oxidized to 2-oxo-histidine,
one or more tryptophans are deoxidated;
one or more asparagines thereof are glycosylated;
one or more serines or threonines are O-glycosylated;
one or more asparagines are deamidated;
one or more Aspartate-Glycine motifs are converted to iso-aspartate-glycine and/or Asn-Gly;
one or more methionines are oxidized;
one or more tryptophans are converted to N-formylkynurenin;
one or more arginines are converted to Arg 3-deoxyglucosone;
the C-terminal residue is not present;
there are one or more non-glycosylated glycosites;
is xylosylated;
is glycated at a lysine;
comprises a cystine with a free-thiol group;
comprises an intrachain disulfide bridge;
comprises disulfide bridges in parallel or crossed orientation; and/or
comprises a lysine or arginine which is carboxymethylated.

8. The VEGF mini-trap of claim 1 which is a homodimer.

9. A composition comprising a heterogeneous mixture of glycosylated variants of the VEGF mini-trap of claim 1.

10. The composition of claim 9, wherein about 20-27% of the VEGF mini-traps are sialylated, about 64-72% of the VEGF mini-traps are galactosylated, about 43-45% of the VEGF mini-traps are fucosylated, about 19-25% of the VEGF mini-traps are Man-5 glycosylated, and/or about 1-2% modified with bisecting N-glycan.

11. A composition comprising a VEGF mini-trap of claim 1; optionally, further comprising IdeS protease or a variant thereof.

12. A complex comprising the VEGF mini-trap homodimer of claim 1 bound to VEGF or a homodimer thereof.

13. The complex of claim 12:
wherein VEGF is human VEGF;
wherein VEGF is human VEGF₁₆₅;
wherein VEGF is human VEGF₁₂₁;
which includes an anti-VEGF antibody or Fab fragment thereof;
which includes human VEGF homodimer and an anti-VEGF Fab fragment wherein the stoichiometric ratio of VEGF mini-trap homodimer:human VEGF:Fab is 1:1:2;
and/or,
wherein the stoichiometric ratio of VEGF mini-trap homodimer:VEGF homodimer is 1:1.

14. A pharmaceutical formulation comprising the VEGF mini-trap of claim 1 and a pharmaceutically effective carrier.

15. An isolated polynucleotide that encodes the VEGF mini-trap of claim 1, optionally comprising the nucleotide sequence set forth in SEQ ID NO: 71, 72 or 73.

16. A vector comprising the polynucleotide of claim 15.

17. A host cell comprising the vector of claim 16.

18. The host cell of claim 17 which is a Chinese hamster ovary cell.

19. An isolated polypeptide comprising, consisting or consisting essentially an amino acid sequence selected from the group consisting of SEQ ID Nos: 32-49.

20. A method for making a VEGF mini-trap or pharmaceutical formulation thereof comprising contacting a VEGF trap of claim 2 with a protease that cleaves the MC of said VEGF trap and incubating the VEGF trap and protease under conditions that promotes cleavage, and, optionally, isolating the VEGF mini-trap product of the cleavage.

21. The method of claim 20, wherein the protease is IdeS.

22. The method of claim 21, wherein the IdeS protease comprises an amino acid sequence selected from SEQ ID Nos: 50-65.

23. A method for making a VEGF mini-trap comprising incubating a host cell of claim 17 in a culture medium under conditions that promote expression of the mini-trap and, optionally, isolating the mini-trap from the host cell and/or medium.

24. A VEGF mini-trap that is the product of a method of claim 23.

25. The VEGF mini-trap of claim 24 which is a homodimer.

26. A vessel, bioreactor or injection device comprising a VEGF mini-trap claim 1 or a pharmaceutical formulation thereof.

27. The vessel, bioreactor or injection device of claim 26 which is an injection device selected from a syringe, a disposable syringe and a pre-filled syringe.

28. The injection device of claim 26 which is sterile and/or glass and/or plastic.

29. A method for treating or preventing an angiogenic eye disorder, in a subject in need thereof, comprising administering a therapeutically effective amount of VEGF mini-trap claim 1 or a pharmaceutical formulation thereof to the subject.

30. The method of claim 29, wherein the angiogenic eye disorder is age-related macular degeneration, wet age-related macular degeneration, macular edema, macular edema following retinal vein occlusion, retinal vein occlusion (RVO), central retinal vein occlusion (CRVO), branch retinal vein occlusion (BRVO), diabetic macular edema (DME), choroidal neovascularization (CNV), iris neovascularization, neovascular glaucoma, retinopathy of prematurity (ROP), post-surgical fibrosis in glaucoma, proliferative vitreoretinopathy (PVR), optic disc neovascularization, corneal neovascularization, retinal neovascularization, vitreal neovascularization, pannus, pterygium, vascular retinopathy, diabetic retinopathy in a subject with diabetic macular edema; diabetic retinopathy, non-proliferative diabetic retinopathy, and/or proliferative diabetic retinopathy.

31. The method of claim 29, wherein the VEGF mini-trap is administered to the subject intraocularly.

32. The method of claim 29, wherein the VEGF mini-trap is administered to the subject intravitreally.

33. A method for reducing neovascularization of the retina and/or reducing vascular permeability of the retina and/or increasing avascularized area of the retina and/or reducing vascularized area of the retina of a subject in need thereof comprising administering a therapeutically effective amount of VEGF mini-trap of claim 1 or a pharmaceutical formulation thereof to the subject.

34. A method for treating or preventing cancer, in a subject in need thereof, comprising administering a therapeutically effective amount of VEGF mini-trap of claim 1 or a pharmaceutical formulation thereof to the subject.

35. The method of claim 34, wherein the cancer is lung cancer, breast cancer, colorectal cancer, metastatic colorectal cancer, prostate cancer, skin cancer or stomach cancer.

36. A method for administering a VEGF mini-trap of claim 1 or a pharmaceutical formulation thereof, to a subject, comprising introducing the VEGF mini-trap or pharmaceutical formulation into the body of the subject.

37. The method of claim 36, wherein the VEGF mini-trap or pharmaceutical formulation is introduced into the body of the subject by injection.

38. The method of claim 36, wherein the VEGF mini-trap or pharmaceutical formulation is introduced into the eye of the subject by intravitreal injection.

39. The method of claim 36, wherein the subject does not experience an increase in intraocular pressure and/or blood pressure following said administration.
